(19) United States
(12) Patent Application Publication
(10) Pub. No.: US 2023/0113157 A1
(43) Pub. Date: Apr. 13, 2023

(54) COMPOSITIONS AND METHODS FOR T CELL ENGINEERING

(71) Applicant: GRACELL BIOTECHNOLOGIES (SHANGHAI) CO., LTD., Shanghai (CN)

(72) Inventors: Jianning GE, Shanghai (CN); Chunhui YANG, Shanghai (CN); Xinxin WANG, Shanghai (CN); Hua ZHANG, Shanghai (CN); Lianjun SHEN, Shanghai (CN)

(21) Appl. No.: 17/042,499

(22) PCT Filed: Sep. 27, 2020
(86) PCT No.: PCT/CN2020/118085

§ 371 Date: Sep. 28, 2020

(30) Foreign Application Priority Data
May 6, 2020 (CN) PCT/CN2020/088734

(51) Int. Cl. A61K 35/17; A61P 35/00

(52) U.S. Cl. CPC A61K 35/17; A61P 35/00

(57) ABSTRACT

The present disclosure relates to an engineered immune cell and use thereof. The present disclosure provides an engineered immune cell comprising a CAR or engineered TCR, which CAR or engineered TCR can comprise a first antigen binding domain and a second antigen binding domain. The engineered immune cells of the present disclosure, when administered into a subject, can inhibit the host immune cells such as T cells and/or NK cells and enhance the survival and persistence of the engineered immune cells in vivo, thereby exhibiting more effective tumor killing activity.

### BACKGROUND

The generation of tumor-specific T lymphocytes by genetic modification to express chimeric antigen receptors (CARs) is gaining traction as a form of synthetic biology generating powerful antitumor effects. Because the specificity is conferred by antibody fragments, the CAR-T cells are not MHC restricted and are therefore more practical than approaches based on T-cell receptors that require MHC matching.

B-lymphocyte antigen CD19, also known as CD19 is a transmembrane protein expressed in all B lineage cells. It has been regarded as an attractive target for CAR-modified T-cell therapies. Clinical data obtained in the treatment of cancer by using CD19 targeted CAR-T have shown promising results, and most of the clinical trials use autologous CAR-T cell infusion to prevent graft versus host disease (GVHD). However, some patients' T cells are not sufficiently effective even after CAR redirection, which promotes the modification of allogeneic donor T cells.

### SUMMARY

Recognized herein is a need for compositions and methods for genetically modifying immune cells (e.g., T cells) in cell therapy. Also recognized herein is a need for compositions and methods for CD-19 targeted CAR-T cells while circumventing using patient-specific T cell. The present disclosure addresses these needs and provides additional advantages as well.

In one aspect, provided is an engineered immune cell comprising a chimeric antigen receptor (CAR), wherein the CAR comprises: (i) a first antigen binding domain that specifically binds to CD19, wherein the first antigen binding domain comprises: (a) a VH1 region, wherein the VH1 region comprises an amino acid sequence having at least 90% identity with any one selected the group consisting of SEQ ID NO. 1-10, and (b) a VL1 region, wherein the VL1 region comprises an amino acid sequence having at least 90% identity with any one selected the group consisting of SEQ ID NO. 11-17; (ii) a second antigen binding domain that specifically binds to CD7, and (iii) a transmembrane domain and an intracellular signaling domain.

In some embodiments, the VH1 region comprises an amino acid sequence having at least 95% identity with any one selected the group consisting of SEQ ID NO. 1-10. In some embodiments, the VH1 region comprises an amino acid sequence having at least 99% identity with any one selected the group consisting of SEQ ID NO. 1-10. In some embodiments, the VH1 region comprises an amino acid sequence of any one selected from the group consisting of SEQ ID NO. 1-10.

In some embodiments, the VL1 region comprises an amino acid sequence having at least 95% identity with any one selected the group consisting of SEQ ID NO. 11-17. In some embodiments, the VL1 region comprises an amino acid sequence having at least 99% identity with any one selected the group consisting of SEQ ID NO. 11-17. In some embodiments, the VL1 region comprises an amino acid sequence of any one selected the group consisting of SEQ ID NO. 11-17.

In some embodiments, the first antigen binding domain or the second antigen binding domain is a human or humanized antigen binding domain. In some embodiments, the first antigen binding domain or the second antigen binding domain is a scFv.

In some embodiments, the first antigen binding domain and the second antigen binding domain are arranged, from amino terminus to carboxyl terminus, in one of following patterns: (i) VL2-VH1-VL1-VH2; (ii) VH2-VL1-VH1-VL2; (iii) VL1-VH2-VL2-VH1; (iv) VH1-VL2-VH2-VL1; (v) VL2-VL1-VH1-VH2; (vi) VH2-VH1-VL1-VL2; (vii) VL1-VL2-VH2-VH1; or (viii) VH1-VH2-VL2-VL1; wherein VH1 represents heavy chain variable domain of the first antigen binding domain, VL1 represents light chain variable light domain of the first antigen binding domain, VH2 represents heavy chain variable domain of the second antigen binding domain, and VL2 represents light chain variable domain of the second antigen binding domain.

In some embodiments, the first antigen binding domain and the second antigen binding domain are arranged, from amino terminus to carboxyl terminus, in one of following patterns: (i) VL2-VH2-VL1-VH1; (ii) VL2-VH2-VH1-VL1; (iii) VL1-VH1-VL2-VH2; (iv) VL1-VH1-VH2-VL2; (v) VH2-VL2-VL1-VH1; (vi) VH2-VL2-VH1-VL1; (vii) VH1-VL1-VL2-VH2; or (viii) VH1-VL1-VH2-VL2, wherein VH1 represents heavy chain variable domain of the first antigen binding domain, VL1 represents light chain variable light domain of the first antigen binding domain, VH2 represents heavy chain variable domain of the second antigen binding domain, and VL2 represents light chain variable domain of the second antigen binding domain.

In some embodiments, the CAR further comprises a second transmembrane domain and a second intracellular signaling domain. In some embodiments, the first antigen binding domain is linked to the intracellular signaling domain via the transmembrane domain and the second antigen binding domain is linked to the second intracellular signaling domain via the second transmembrane domain.

In some embodiments, the first or second transmembrane domain comprises at least a portion of TCR alpha, TCR beta, CD3 zeta, CD3 gamma, CD3 delta, CD3 epsilon, CD2, CD7, CD27, CD28, CD45, CD4, CD5, CD8, CD9, CD16, CD22, CD33, CD37, CD64, CD80, CD86, CD134, CD137, CD152, PD-1, or CD154.

In some embodiments, the first or second intracellular signaling domain comprises at least a portion of CD3 zeta, FcR gamma, FcR beta, CD3 gamma, CD3 delta, CD3 epsilon, CD5, CD22, CD79a, CD79b, or CD66d.

In some embodiments, the first or second intracellular signaling domain further comprises a costimulatory domain. In some embodiments, the costimulatory domain is selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83.

In some embodiments, the first antigen binding domain and the second antigen binding domain are linked via a linker. In some embodiments, the linker is a cleavable linker. In some embodiments, the linker is a self-cleaving peptide. In some embodiments, the cleavable linker is selected from P2A, T2A, E2A, and F2A.

In some embodiments, the engineered immune cell is a T cell, an NKT cell or an NK cell. In some embodiments, the T cell is an alpha beta T cell or a gamma delta T cell. In some embodiments, the engineered immune cell is derived from a stem cell. In some embodiments, the stem cell is a hematopoietic stem cell (HSC) or an induced pluripotent stem cell (iPSC). In some embodiments, the engineered immune cell is an autologous cell or an allogeneic cell. In some embodiments, the engineered immune cell is obtained from a subject having a condition. In some embodiments, the engineered immune cell is obtained from a healthy donor.

In some embodiments, an endogenous T cell receptor (TCR) of the engineered immune cell is inactivated. In some embodiments, a gene encoding a subunit of the endogenous TCR is inactivated such that the endogenous TCR is inactivated. In some embodiments, the subunit is selected from TCRα, TCRβ, CD3ε, CD3δ, CD3γ, and CD3ζ.

In some embodiments, an endogenous CD7 of the engineered immune cell is inactivated or suppressed. In some embodiments, a gene encoding the endogenous CD7 of the engineered immune cell is inactivated, or protein expression of the endogenous CD7 of the engineered immune cell is suppressed.

In some embodiments, expression of one or more endogenous human leukocyte antigen (HLA) genes of the cell remains intact. In some embodiments, expression of one or more endogenous human leukocyte antigen (HLA) genes of the cell is inhibited. In some embodiments, expression of one or more endogenous human leukocyte antigen (HLA) genes of the cell is inactivated. In some embodiments, the HLA gene comprises HLA-A, HLA-B, HLA-C, HLA-E, HLA-F, HLA-G, HLA-DRA, HLA-DRB, HLA-DQA and HLA-DQB.

In some embodiments, the engineered immune cell further comprises an enhancer moiety capable of enhancing one or more activities of the engineered immune cell. In some embodiments, the enhancer moiety is configured to constitutively enhance the one or more activities of the engineered immune cell. In some embodiments, the enhancer moiety is configured to constitutively upregulate one or more intracellular signaling pathways of the engineered immune cell. In some embodiments, the one or more intracellular signaling pathways are one or more cytokine signaling pathways. In some embodiments, the enhancer moiety is self-activating through self-oligomerizing. In some embodiments, the enhancer moiety is self-activating through self-dimerizing. In some embodiments, the enhancer moiety is a cytokine or a cytokine receptor. In some embodiments, the enhancer moiety is selected from the group consisting of IL-2, IL-3, IL-4, IL-6, IL-7, IL-8, IL-10, IL-11, IL-12, IL-15, IL-17, IL-18, IL-21, IL-23, PD-1, PD-L1, CD122, CSF1R, CTAL-4, TIM-3, CCL21, CCL19, TGFR beta, receptors for the same, functional fragments thereof, functional variants thereof, and combinations thereof. In some embodiments, the enhancer moiety functions as a trans-activating factor or a cis-activating factor.

In some embodiments, the enhancer is linked to the CAR via a linker. In some embodiments, the linker is a cleavable linker. In some embodiments, the linker is a self-cleaving peptide. In some embodiments, the cleavable linker is selected from P2A, T2A, E2A, and F2A.

In some embodiments, the engineered immune cell further comprises an inducible cell death moiety capable of effecting death of the engineered immune cell upon contacting the chimeric polypeptide with a cell death activator. In some embodiments, the inducible cell death moiety is selected from the group consisting of rapaCasp9, iCasp9, HSV-TK, CD20, ΔCD20, mTMPK, ΔCD19, RQR8, Her2t, CD30, BCMA and EGFRt. In some embodiments, the inducible cell death moiety is EGFRt, and the cell death activator is an antibody or an antigen binding fragment thereof that binds EGFRt. In some embodiments, the inducible cell death moiety is HSV-TK, and the cell death activator is GCV. In some embodiments, the inducible cell death moiety is iCasp9, and the cell death activator is AP1903. In some embodiments, the cell death activator comprises a nucleic acid, a polynucleotide, an amino acid, a polypeptide, lipid, a carbohydrate, a small molecule, an enzyme, a ribosome, a proteasome, a variant thereof, or any combination thereof. In some embodiments, the enhancer moiety is linked to the inducible cell death moiety.

In some embodiments, said engineered immune cell exhibits enhanced viability while in presence of cells that are heterologous to said engineered immune cell.

In another aspect, provided is a composition comprising the engineered immune cell as described above, and a pharmaceutically acceptable excipient.

In another aspect, provided is an isolated polynucleotide encoding the CAR as described above

In another aspect, provided is a method of delivering an allogeneic cell therapy comprising administering to a subject in need thereof a population of engineered immune cells as described above. In some embodiments, an endogenous TCR of the engineered immune cell is functionally inactive. In some embodiments, the cell reduces GvHD in the subject compared to an additional cell having a functionally active TCR.

In another aspect, provided is a method for treating cancer, comprising administering to a patient in need thereof a population of the engineered immune cells as described above. In some embodiments, the cancer is lymphoma or leukemia.

In another aspect, provided is a kit comprising the engineered immune cells or the isolated polynucleotide as described above, and an instruction for using the kit.

In another aspect, provided is an engineered immune cell comprising a chimeric antigen receptor (CAR), wherein the CAR comprises: (i) a humanized first antigen binding domain that specifically binds to CD19, wherein the first antigen binding domain comprises: (a) a VH1 region, wherein the VH1 region comprises an amino acid sequence having a structure of formula I:

FH1-X1-FH2-X2-FH3-X3-FH4 (I)

wherein —FH1 comprises an amino acid identical to or comprising 1-3 amino acid residue addition, deletion, or substitutions relative to SEQ ID NO. 18; —FH2 comprises an amino acid identical to or comprising 1-3 amino acid residue addition, deletion, or substitutions relative to SEQ ID NO. 19; —FH3 comprises an amino acid identical to or comprising 1-3 amino acid residue addition, deletion, or substitutions relative to SEQ ID NO. 20; —FH4 comprises an amino acid identical to or comprising 1-3 amino acid residue addition, deletion, or substitutions relative to SEQ ID NO. 21; and —X1, X2, and X3 represent heavy chain CDRs of the first antigen binding domain; and (b) a VL1 region, wherein the VH1 region comprises an amino acid sequence having a structure of formula II:

FL1-Y1-FL2-Y2-FL3-Y3-FL4 (II)

wherein —FL1 comprises an amino acid identical to or comprising 1-3 amino acid residue addition, deletion, or substitutions relative to SEQ ID NO. 22; —FL2 comprises an amino acid identical to or comprising 1-3 amino acid residue addition, deletion, or substitutions relative to SEQ ID NO. 23; —FL3 comprises an amino acid identical to or comprising 1-3 amino acid residue addition, deletion, or substitutions relative to SEQ ID NO. 24; —FL4 comprises an amino acid identical to or comprising 1-3 amino acid residue addition, deletion, or substitutions relative to SEQ ID NO. 25; and —Y1, Y2, and Y3 represent light chain CDRs of the first antigen binding domain; (ii) a second antigen binding domain that specifically binds to CD7; and (iii) a transmembrane domain and an intracellular signaling domain.

In some embodiments, FH1 is selected from a sequence of SEQ ID NO. 18, 34, 35 or 36.

In some embodiments, FH2 is selected from a sequence of SEQ ID NO. 19 or 37.

In some embodiments, FH3 is selected from a sequence of SEQ ID NO. 20, 38, 39, 40, 41, 42, 43 or 44.

In some embodiments, FH4 is selected from a sequence of SEQ ID NO. 21 or 45.

In some embodiments, FL1 is selected from a sequence of SEQ ID NO. 22 or 46.

In some embodiments, FL2 is selected from a sequence of SEQ ID NO. 23, 47 or 48.

In some embodiments, FL3 is selected from a sequence of SEQ ID NO. 24, 49, 50, 51 or 52.

In some embodiments, FL4 is a sequence of SEQ ID NO. 25.

In some embodiments, the FH1 comprises an amino acid identical to or comprising 1-3 amino acid residue addition, deletion, or substitutions relative to SEQ ID NO. 34, the FH2 comprises an amino acid identical to or comprising 1-3 amino acid residue addition, deletion, or substitutions relative to SEQ ID NO. 37, the FH3 comprises an amino acid identical to or comprising 1-3 amino acid residue addition, deletion, or substitutions relative to SEQ ID NO. 38, the FH4 comprises an amino acid identical to or comprising 1-3 amino acid residue addition, deletion, or substitutions relative to SEQ ID NO. 45.

In some embodiments, the FH1 comprises an amino acid identical to or comprising 1-3 amino acid residue addition, deletion, or substitutions relative to SEQ ID NO. 34, the FH2 comprises an amino acid identical to or comprising 1-3 amino acid residue addition, deletion, or substitutions relative to SEQ ID NO. 37, the FH3 comprises an amino acid identical to or comprising 1-3 amino acid residue addition, deletion, or substitutions relative to SEQ ID NO. 39, the FH4 comprises an amino acid identical to or comprising 1-3 amino acid residue addition, deletion, or substitutions relative to SEQ ID NO. 45.

In some embodiments, the FH1 comprises an amino acid identical to or comprising 1-3 amino acid residue addition, deletion, or substitutions relative to SEQ ID NO. 34, the FH2 comprises an amino acid identical to or comprising 1-3 amino acid residue addition, deletion, or substitutions relative to SEQ ID NO. 37, the FH3 comprises an amino acid identical to or comprising 1-3 amino acid residue addition, deletion, or substitutions relative to SEQ ID NO. 40, the FH4 comprises an amino acid identical to or comprising 1-3 amino acid residue addition, deletion, or substitutions relative to SEQ ID NO. 45.

In some embodiments, the FH1 comprises an amino acid identical to or comprising 1-3 amino acid residue addition, deletion, or substitutions relative to SEQ ID NO. 34, the FH2 comprises an amino acid identical to or comprising 1-3 amino acid residue addition, deletion, or substitutions relative to SEQ ID NO. 19, the FH3 comprises an amino acid identical to or comprising 1-3 amino acid residue addition, deletion, or substitutions relative to SEQ ID NO. 40, the FH4 comprises an amino acid identical to or comprising 1-3 amino acid residue addition, deletion, or substitutions relative to SEQ ID NO. 45.

In some embodiments, the FH1 comprises an amino acid identical to or comprising 1-3 amino acid residue addition, deletion, or substitutions relative to SEQ ID NO. 35, the FH2 comprises an amino acid identical to or comprising 1-3 amino acid residue addition, deletion, or substitutions relative to SEQ ID NO. 19, the FH3 comprises an amino acid identical to or comprising 1-3 amino acid residue addition, deletion, or substitutions relative to SEQ ID NO. 41, the FH4 comprises an amino acid identical to or comprising 1-3 amino acid residue addition, deletion, or substitutions relative to SEQ ID NO. 21.

In some embodiments, the FH1 comprises an amino acid identical to or comprising 1-3 amino acid residue addition, deletion, or substitutions relative to SEQ ID NO. 18, the FH2 comprises an amino acid identical to or comprising 1-3 amino acid residue addition, deletion, or substitutions relative to SEQ ID NO. 19, the FH3 comprises an amino acid identical to or comprising 1-3 amino acid residue addition, deletion, or substitutions relative to SEQ ID NO. 20, the FH4 comprises an amino acid identical to or comprising 1-3 amino acid residue addition, deletion, or substitutions relative to SEQ ID NO. 21.

In some embodiments, the FH1 comprises an amino acid identical to or comprising 1-3 amino acid residue addition, deletion, or substitutions relative to SEQ ID NO. 18, the FH2 comprises an amino acid identical to or comprising 1-3 amino acid residue addition, deletion, or substitutions relative to SEQ ID NO. 37, the FH3 comprises an amino acid identical to or comprising 1-3 amino acid residue addition, deletion, or substitutions relative to SEQ ID NO. 42, the FH4 comprises an amino acid identical to or comprising 1-3 amino acid residue addition, deletion, or substitutions relative to SEQ ID NO. 21.

In some embodiments, the FH1 comprises an amino acid identical to or comprising 1-3 amino acid residue addition, deletion, or substitutions relative to SEQ ID NO. 18, the FH2 comprises an amino acid identical to or comprising 1-3 amino acid residue addition, deletion, or substitutions relative to SEQ ID NO. 36, the FH3 comprises an amino acid identical to or comprising 1-3 amino acid residue addition, deletion, or substitutions relative to SEQ ID NO. 43, the FH4 comprises an amino acid identical to or comprising 1-3 amino acid residue addition, deletion, or substitutions relative to SEQ ID NO. 21.

In some embodiments, the FH1 comprises an amino acid identical to or comprising 1-3 amino acid residue addition, deletion, or substitutions relative to SEQ ID NO. 18, the FH2 comprises an amino acid identical to or comprising 1-3 amino acid residue addition, deletion, or substitutions relative to SEQ ID NO. 19, the FH3 comprises an amino acid identical to or comprising 1-3 amino acid residue addition, deletion, or substitutions relative to SEQ ID NO. 44, the FH4 comprises an amino acid identical to or comprising 1-3 amino acid residue addition, deletion, or substitutions relative to SEQ ID NO. 21.

In some embodiments, the FL1 comprises an amino acid identical to or comprising 1-3 amino acid residue addition, deletion, or substitutions relative to SEQ ID NO. 22, the FL2 comprises an amino acid identical to or comprising 1-3 amino acid residue addition, deletion, or substitutions relative to SEQ ID NO. 47, the FL3 comprises an amino acid identical to or comprising 1-3 amino acid residue addition, deletion, or substitutions relative to SEQ ID NO. 49 and the FL4 comprises an amino acid identical to or comprising 1-3 amino acid residue addition, deletion, or substitutions relative to SEQ ID NO. 25.

In some embodiments, the FL1 comprises an amino acid identical to or comprising 1-3 amino acid residue addition, deletion, or substitutions relative to SEQ ID NO. 22, the FL2 comprises an amino acid identical to or comprising 1-3 amino acid residue addition, deletion, or substitutions relative to SEQ ID NO. 47, the FL3 comprises an amino acid identical to or comprising 1-3 amino acid residue addition, deletion, or substitutions relative to SEQ ID NO. 50 and the FL4 comprises an amino acid identical to or comprising 1-3 amino acid residue addition, deletion, or substitutions relative to SEQ ID NO. 25.

In some embodiments, the FL1 comprises an amino acid identical to or comprising 1-3 amino acid residue addition, deletion, or substitutions relative to SEQ ID NO. 22, the FL2 comprises an amino acid identical to or comprising 1-3 amino acid residue addition, deletion, or substitutions relative to SEQ ID NO. 47, the FL3 comprises an amino acid identical to or comprising 1-3 amino acid residue addition, deletion, or substitutions relative to SEQ ID NO. 51 and the FL4 comprises an amino acid identical to or comprising 1-3 amino acid residue addition, deletion, or substitutions relative to SEQ ID NO. 25.

In some embodiments, the FL1 comprises an amino acid identical to or comprising 1-3 amino acid residue addition, deletion, or substitutions relative to SEQ ID NO. 46, the FL2 comprises an amino acid identical to or comprising 1-3 amino acid residue addition, deletion, or substitutions relative to SEQ ID NO. 47, the FL3 comprises an amino acid identical to or comprising 1-3 amino acid residue addition, deletion, or substitutions relative to SEQ ID NO. 24 and the FL4 comprises an amino acid identical to or comprising 1-3 amino acid residue addition, deletion, or substitutions relative to SEQ ID NO. 25.

In some embodiments, the FL1 comprises an amino acid identical to or comprising 1-3 amino acid residue addition, deletion, or substitutions relative to SEQ ID NO. 22, the FL2 comprises an amino acid identical to or comprising 1-3 amino acid residue addition, deletion, or substitutions relative to SEQ ID NO. 23, the FL3 comprises an amino acid identical to or comprising 1-3 amino acid residue addition, deletion, or substitutions relative to SEQ ID NO. 24 and the FL4 comprises an amino acid identical to or comprising 1-3 amino acid residue addition, deletion, or substitutions relative to SEQ ID NO. 25.

In some embodiments, the FL1 comprises an amino acid identical to or comprising 1-3 amino acid residue addition, deletion, or substitutions relative to SEQ ID NO. 46, the FL2 comprises an amino acid identical to or comprising 1-3 amino acid residue addition, deletion, or substitutions relative to SEQ ID NO. 48, the FL3 comprises an amino acid identical to or comprising 1-3 amino acid residue addition, deletion, or substitutions relative to SEQ ID NO. 52 and the FL4 comprises an amino acid identical to or comprising 1-3 amino acid residue addition, deletion, or substitutions relative to SEQ ID NO. 25.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1A illustrates exemplary designs of CD7/CD19 dual CARs in loop structure.

FIG. 1B illustrates exemplary designs of CD7/CD19 dual CARs in parallel structure.

FIG. 2A and FIG. 2B illustrate expression of the CD7/CD19 dual CARs in loop structure. FIG. 2C illustrates the knockout efficiencies of CD7 and CD3 (TRAC) in T cells.

FIG. 3A and FIG. 3B illustrate in vitro killing effects of loop structural CD7/CD19 dual CAR-T cells on CD19+ cells and CD7+ cells.

FIG. 4A and FIG. 4B illustrate expression of CD7/CD19 dual CARs in parallel structure together with the C7R enhancer.

FIG. 5A, FIG. 5B and FIG. 5C illustrates killing potency of CD7/CD19 dual CAR-T cells.

FIG. 6 illustrates release of the cytokines by CD7/CD19 dual CAR-T cells.

FIG. 7 illustrates delayed progression of CCRF-CEM tumors in vivo by CD7/CD19 dual CAR-T cells.

FIG. 8 illustrates tissue distribution of CD7 expression.

FIG. 9 illustrates tissue distribution of CD2 expression.

### DETAILED DESCRIPTION

### Definition

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “about,” as used herein, refers to a value or composition within a range of acceptable tolerances for a particular value or composition as determined, which will depend in part on how the value or composition is measured or measured, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The term “administering,” as used herein, refers to physically introducing a product of the present disclosure into a subject using any of a variety of methods and delivery systems, including intravenous, intramuscular, subcutaneous, intraperitoneal, spinal or other routes of parenteral administration, for example by injection or infusion.

The term “antigen,” as used herein, refers to a molecule or a fragment thereof capable of being bound by a selective binding agent. As an example, an antigen can be a ligand that can be bound by a selective binding agent such as a receptor. As another example, an antigen can be an antigenic molecule that can be bound by a selective binding agent such as an immunological protein (e.g., an antibody). An antigen can also refer to a molecule or fragment thereof capable of being used in an animal to produce antibodies capable of binding to that antigen. In some cases, an antigen may be bound to a substrate (e.g., a cell membrane). Alternatively, an antigen may not be bound to a substrate (e.g., a secreted molecule, such as a secreted polypeptide).

The term “antibody (Ab),” as used herein, include, but is not limited to, an immunoglobulin that specifically binds to an antigen and comprises at least two heavy (H) chains and two light (L) chains interconnected by disulfide bonds, or an antigen thereof. Each H chain comprises a heavy chain variable region (abbreviated herein as VH) and a heavy chain constant region. The heavy chain constant region comprises three constant domains CH1, CH2 and CH3. Each light chain comprises a light chain variable region (abbreviated herein as VL) and a light chain constant region. The light chain constant region comprises a constant domain CL. The VH and VL regions can be further subdivided into hypervariable regions called complementarity determining regions (CDRs) interspersed with more conserved regions called framework regions (FR). Each VH and VL contains three CDRs and four FRs, arranged from the amino terminus to the carboxy terminus in the following order: FR1, CDR1, FR2, CDR2, FR3, CDR3, FR4. The variable regions of the heavy and light chains contain a binding domain that interacts with the antigen.

The term “nucleotide,” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS] dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide can be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides can include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif.; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically-modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three dimensional structure, and can perform any function, known or unknown. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The term “gene,” as used herein, refers to a nucleic acid (e.g., DNA such as genomic DNA and cDNA) and its corresponding nucleotide sequence that is involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters.

The term “percent (%) identity,” as used herein, refers to the percentage of amino acid (or nucleic acid) residues of a candidate sequence that are identical to the amino acid (or nucleic acid) residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment, for purposes of determining percent identity, can be achieved in various ways that are within the skill in the art, for instance, using publicly available computer software such as BLAST, ALIGN, or Megalign (DNASTAR) software. Percent identity of two sequences can be calculated by aligning a test sequence with a comparison sequence using BLAST, determining the number of amino acids or nucleotides in the aligned test sequence that are identical to amino acids or nucleotides in the same position of the comparison sequence, and dividing the number of identical amino acids or nucleotides by the number of amino acids or nucleotides in the comparison sequence.

The term “functionally inactivate” or “functional inactivation” as used herein refers to that a functional gene or the product of the gene such as mRNA or protein is prevented or inhibited. The inactivation may be achieved by deletion, addition or substitution of the gene or the promoter thereof, so that expression does not occur, or mutation of the coding sequence of the gene so that the gene product such as mRNA or protein is inactive. The functional inactivation may be complete or partial. Inactivation of a gene can encompass all degrees of inactivation, including gene silencing, knockout, inhibition and disruption. In some embodiments, the functional inactivation is introduced by CRISPR-Cas9 system.

The term “endogenous,” as used herein, refers to a nucleic acid molecule or polypeptide normally expressed in a cell or tissue.

The term “exogenous,” as used herein, refers to the nucleic acid molecule or polypeptide is not endogenously present in the cell or is present at a level sufficient to achieve the functional effects obtained upon overexpression. Thus, the term “exogenous” includes any recombinant nucleic acid molecule or polypeptide expressed in a cell, e.g., a foreign, heterologous, and overexpressed nucleic acid molecule and polypeptide.

The term “autologous” as used herein, refers to origination from the same being. For example, an autologous sample (e.g., cells) can refer to a sample which is removed, processed, and then given back to the same subject (e.g., patient) at a later time. Autologous, with respect to a process, can be distinguished from an allogenic process in which the donor of a sample (e.g., cells) and the recipient of the sample are not the same subject.

The term “allogeneic” as used herein, refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” as used herein, refers to a graft derived from an animal of a different species.

The term “T cell and NK cell consensus marker,” as used herein, refers to a marker co-existing on T cells and NK cells, including but not limited to: CD2, CD3 CD7, CD38, CD45, CD48, CD50, CD52, CD56, CD69, CD100, CD122, CD132, CD161, CD159a, CD159c, CD314.

The term “marker of T cells and/or NK cells,” as used herein, refers to markers present in T cells or NK cells, respectively, or both T cells and NK cells, including but not limited to: CD2, CD3, CD4, CD5, CD7, CD8, CD16a, CD16b, CD25, CD27, CD28, CD38, CD45, CD48, CD50, CD52, CD56, CD57, CD62L, CD69, CD94, CD100, CD102, CD122, CD127, CD132, CD160, CD161 CD178, CD218, CD226, CD244, CD159a (NKG2A), CD159c (NKG2C), NKG2E, CD314 (NKG2D), CD305, CD335 (NKP46), CD337, SLAMF7.

The terms “subject,” “individual,” and “patient” are used interchangeably herein to refer to a vertebrate, preferably a mammal such as a human. Mammals include, but are not limited to, murines, simians, humans, farm animals, sport animals, and pets. Tissues, cells and their progeny of a biological entity obtained in vivo or cultured in vitro are also encompassed.

The terms “treatment” and “treating,” as used herein, refer to an approach for obtaining beneficial or desired results including but not limited to a therapeutic benefit and/or a prophylactic benefit. For example, a treatment can comprise administering a system or cell population disclosed herein. By therapeutic benefit is meant any therapeutically relevant improvement in or effect on one or more conditions (e.g., diseases or symptoms) under treatment. For prophylactic benefit, a composition can be administered to a subject at risk of developing a particular condition, or to a subject reporting one or more of the physiological symptoms of a disease, even though the condition may not have yet been manifested.

### Overview

CARs can comprise an extracellular antigen recognition region, for example, a scFv (single-chain variable fragment), a transmembrane region, and an intracellular costimulatory signal region. The extracellular domain of CARs can recognize a specific antigen and then transduce the signal through the intracellular domain, causing T cell activation and proliferation, cytolysis toxicity, and secretion of cytokines, thereby eliminating target cells. The patient's autologous T cells (or heterologous donors) can be first isolated, activated and genetically engineered to produce CAR-T cells, which can be then injected into the same patient. In this way, the probability of graft-versus-host disease may be reduced, and the antigen can be recognized by T cells in a non-MHC-restricted manner. In addition, a CAR-T can treat all cancers that express the antigen.

The present disclosure provides compositions and methods to engineer a cell, e.g., an immune cell, such that it can target both disease-associated antigen CD19 and immune cell antigen (e.g., CD2, CD3, CD5 or CD7) through bispecific or multivalent CAR(s). For example, the present disclosure provides an engineered immune cell that can target CD19 and an immune cell antigen such as CD7. The endogenous TCR can be inactivated (e.g., disrupted, inhibited, knocked out or silenced). The CAR-T of the present disclosure which targets CD19 and the immune cell antigen can eliminate CD-19 positive tumor cells and clear host immune cell antigen positive T and NK cells, thereby avoiding host rejection (HVG). In the present disclosure, the endogenous TCR of the engineered immune cell can be knocked out, and graft-versus-host disease (GVHD) can be prevented, thereby preparing a general-purpose or universal CAR-T (UCAR-T) cell. The engineered immune cell can be derived from an autologous T cell or an allogeneic T cell.

Moreover, the engineered immune cell can comprise a cell suicide element (e.g., inducible cell death moiety), and the CAR-T can be inactivated/cleared at any time to reduce side effects. In some cases, the engineered immune cell can further comprise an enhancer moiety. The enhancer moiety can regulate one or more activities of the engineered immune cell when the engineered immune cell is administered to a subject. For example, the enhancer moiety can be a cytokine (e.g., IL-2 or IL-7) or a cytokine receptor (e.g., IL-2R or IL-7R). The enhancer moiety can enhance a signaling pathway within the engineered immune cell, for example, STAT5 signaling pathway. In some embodiments, the engineered immune cell comprises a bispecific CAR targeting both CD19 and CD7. The engineered immune cell show in this example can further comprise an inducible cell death moiety such as a truncated epidermal growth factor receptor (EGFRt or tEGFR, which can be used interchangeably herein; see U.S. Pat. No. 9,447,194B2 and PCT Publication No. WO2018038945).

The inducible cell death moiety or the enhancer moiety can be introduced in the immune cell via a separate expression vector. In some cases, the inducible cell death moiety and the enhancer moiety may be introduced into the immune cell via an expression vector comprising sequences encoding both moieties. In some cases, the inducible cell death moiety and the enhancer moiety are linked and are expressed as a chimeric polypeptide.

The application of the engineered immune cells provided herein in cell therapy can treat the disease (e.g., cancer) of a patient, be prepared in large-scale in advance to avoid GVHD and HvG, reduce treatment costs, inactivate CAR-T at any time if necessary, reduce side effects of immunotherapy, and ensure product safety. The engineered cells provided herein can be referred to as universal CD-19 CAR T cells (UCAR-T cells).

### Chimeric Antigen Receptor (CAR)

The cell (e.g., immune cell or engineered immune cell) provided herein can comprise one or more CARs. The CAR can include an extracellular domain, a transmembrane domain, and an intracellular signaling domain. The extracellular domain can include a target-specific binding element (also known as an antigen binding domain). The intracellular domain can include a costimulatory signaling region and a zeta (ζ) chain portion. A costimulatory signaling region refers to a portion of the CAR comprising the intracellular domain of a costimulatory molecule. Costimulatory molecules are cell surface molecules other than antigens receptors or their ligands that may be needed for an efficient response of lymphocytes to antigen. Between the extracellular domain and the transmembrane domain of the CAR, or between the cytoplasmic domain and the transmembrane domain of the CAR, there may be incorporated a spacer domain. As used herein, the term “spacer domain” generally means any oligo- or polypeptide that functions to link the transmembrane domain to, either the extracellular domain or, the cytoplasmic domain in the polypeptide chain. A spacer domain may comprise up to 300 amino acids, preferably 10 to 100 amino acids and most preferably 25 to 50 amino acids.

With respect to the transmembrane domain, the CAR can be designed to comprise a transmembrane domain that is fused to the extracellular domain of the CAR. In one embodiment, the transmembrane domain that naturally is associated with one of the domains in the CAR is used. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in the present disclosure may be derived from (e.g., comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, or from an immunoglobulin such as IgG4. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

The cytoplasmic domain or otherwise the intracellular signaling domain of the CAR of the present disclosure can be responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus, the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Examples of intracellular signaling domains for use in the CAR of the present disclosure include the cytoplasmic sequences of the TCR and co-receptors that act in concert to initiate signal transduction following antigen receptor engagement, as well as any derivative or variant of these sequences and any synthetic sequence that has the same functional capability.

Signals generated through the TCR alone may be insufficient for full activation of the T cell and that a secondary or co-stimulatory signal may be included. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences can regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences that are of particular use in the present disclosure include those derived from TCR zeta, FcR gamma, FcR beta, CD3 gamma, CD3 delta, CD3 epsilon, CD5, CD22, CD79a, CD79b, and CD66d. It is particularly preferred that cytoplasmic signaling molecule in the CAR of the present disclosure comprises a cytoplasmic signaling sequence derived from CD3-zeta.

In some embodiments, the cytoplasmic domain of the CAR can be designed to comprise the CD3-zeta signaling domain by itself or combined with any other desired cytoplasmic domain(s) useful in the context of the CAR of the present disclosure. For example, the cytoplasmic domain of the CAR can comprise a CD3 zeta chain portion and a costimulatory signaling region. The costimulatory signaling region refers to a portion of the CAR comprising the intracellular domain of a costimulatory molecule. A costimulatory molecule is a cell surface molecule other than an antigen receptor or its ligands that may be needed for an efficient response of lymphocytes to an antigen. Examples of such molecules include CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83, and the like. Thus, while the present disclosure is, in some cases, exemplified with 4-1BB as the co-stimulatory signaling element, other costimulatory elements are within the scope of the present disclosure.

The cytoplasmic signaling sequences within the cytoplasmic signaling portion of the CAR of the present disclosure may be linked to each other in a random or specified order. Optionally, a short oligo- or polypeptide linker, preferably between 2 and 10 amino acids in length may form the linkage. A glycine-serine doublet provides a particularly suitable linker.

In some embodiments, the cytoplasmic domain is designed to comprise the signaling domain of CD3-zeta and the signaling domain of CD28. In another embodiment, the cytoplasmic domain is designed to comprise the signaling domain of CD3-zeta and the signaling domain of 4-1BB. In yet another embodiment, the cytoplasmic domain is designed to comprise the signaling domain of CD3-zeta and the signaling domain of CD28 and 4-1BB.

A CAR provided herein can comprise one or more antigen binding domains. In some cases, a CAR provided herein comprises an antigen binding domain that can target both an immune cell antigen (e.g., to inhibit killing activity of a T cell or NK cell) and a disease-associated antigen (e.g., a tumor-associated antigen). For example, an antigen binding domain targeting both immune cell antigens and cancer antigens include, but not limited to, CD2, CD3, CD5, and CD7. In some cases, a CAR provided herein comprises two antigen binding domains such that one individual CAR is a bispecific CAR, targeting two different antigens. For bispecific CAR, one antigen binding domain can target immune cell antigen, and the other antigen binding domain can target disease-associated antigen. In some embodiments, one antigen binding domain can target immune cell antigen CD7, and the other antigen binding domain can target disease-associated antigen CD19. In some embodiments, one antigen binding domain can target immune cell antigen CD2, and the other antigen binding domain can target disease-associated antigen CD19. In some embodiments, one antigen binding domain can target immune cell antigen CD3, and the other antigen binding domain can target disease-associated antigen CD19. In some embodiments, one antigen binding domain can target immune cell antigen CD5, and the other antigen binding domain can target disease-associated antigen CD19. The two antigen binding domains of a bispecific CAR can have a tandem structure, a parallel structure or a loop structure.

In some cases, a CAR can target CD19 and CD7. The CAR can have a structure as formula I: L-scFv1-I-scFv2-H-TM-C-CD3ζ (I), wherein each “-” is independently a linker peptide or a peptide bond; L is optionally a signaling peptide sequence; I is a flexible linker; H is optionally a hinge region; TM is a transmembrane domain; C is a costimulatory domain; CD3ζ is a cytoplasmic signaling sequence derived from CD3; one of scFv1 and scFv2 is an antigen binding domain targeting CD19, and the other one is an antigen binding domain targeting CD7. The CAR can have a structure as formula II or II′: L-VL-scFv-VH-H-TM-C-CD3ζ (II), L-VH-scFv-VL-H-TM-C-CD3ζ (II′), wherein each “-” is independently a linker peptide or a peptide bond; the elements L, H, TM, C and CD3 as described above; scFv is an antigen binding domain targeting CD19, VH is an anti-CD7 antibody heavy chain variable region, and VL is an anti-CD7 antibody light chain variable region; or scFv is an antigen binding domain targeting CD-7, VH is an anti-CD19 antibody heavy chain variable region, and VL is an anti-CD19 antibody light chain variable region.

In some cases, a CAR can target CD19 and CD2. The CAR can have a structure as formula I: L-scFv1-I-scFv2-H-TM-C-CD3ζ (I), wherein each “-” is independently a linker peptide or a peptide bond; L is optionally a signaling peptide sequence; I is a flexible linker; H is optionally a hinge region; TM is a transmembrane domain; C is a costimulatory domain; CD3ζ is a cytoplasmic signaling sequence derived from CD3; one of scFv1 and scFv2 is an antigen binding domain targeting CD19, and the other one is an antigen binding domain targeting CD2. The CAR can have a structure as formula II or II′: L-VL-scFv-VH-H-TM-C-CD3ζ (II), L-VH-scFv-VL-H-TM-C-CD3ζ (II′), wherein each “-” is independently a linker peptide or a peptide bond; the elements L, H, TM, C and CD3 as described above; scFv is an antigen binding domain targeting a CD19, VH is an anti-CD2 antibody heavy chain variable region, and VL is an anti-CD2 antibody light chain variable region; or scFv is an antigen binding domain targeting CD-2, VH is an anti-CD19 antibody heavy chain variable region, and VL is an anti-CD19 antibody light chain variable region.

In some cases, a CAR can target CD19 and CD3. The CAR can have a structure as formula I: L-scFv1-I-scFv2-H-TM-C-CD3ζ (I), wherein each “-” is independently a linker peptide or a peptide bond; L is optionally a signaling peptide sequence; I is a flexible linker; H is optionally a hinge region; TM is a transmembrane domain; C is a costimulatory domain; CD3ζ is a cytoplasmic signaling sequence derived from CD3; one of scFv1 and scFv2 is an antigen binding domain targeting CD19, and the other one is an antigen binding domain targeting CD3. The CAR can have a structure as formula II or II′: L-VL-scFv-VH-H-TM-C-CD3ζ (II), L-VH-scFv-VL-H-TM-C-CD3ζ (II′), wherein each “-” is independently a linker peptide or a peptide bond; the elements L, H, TM, C and CD3 as described above; scFv is an antigen binding domain targeting a CD19, VH is an anti-CD3 antibody heavy chain variable region, and VL is an anti-CD3 antibody light chain variable region; or scFv is an antigen binding domain targeting CD-3, VH is an anti-CD 19 antibody heavy chain variable region, and VL is an anti-CD 19 antibody light chain variable region.

In some cases, a CAR can target CD19 and CD5. The CAR can have a structure as formula I: L-scFv1-I-scFv2-H-TM-C-CD3ζ (I), wherein each “-” is independently a linker peptide or a peptide bond; L is optionally a signaling peptide sequence; I is a flexible linker; H is optionally a hinge region; TM is a transmembrane domain; C is a costimulatory domain; CD3ζ is a cytoplasmic signaling sequence derived from CD3ζ; one of scFv1 and scFv2 is an antigen binding domain targeting CD19, and the other one is an antigen binding domain targeting CD5. The CAR can have a structure as formula II or II′: L-VL-scFv-VH-H-TM-C-CD3ζ (II), L-VH-scFv-VL-H-TM-C-CD3ζ (II′), wherein each “-” is independently a linker peptide or a peptide bond; the elements L, H, TM, C and CD3ζ as described above; scFv is an antigen binding domain targeting a CD19, VH is an anti-CD5 antibody heavy chain variable region, and VL is an anti-CD5 antibody light chain variable region; or scFv is an antigen binding domain targeting CD-5, VH is an anti-CD 19 antibody heavy chain variable region, and VL is an anti-CD 19 antibody light chain variable region.

In some cases, a CAR can comprise the structure of EGFRt-CD7 scFv-CD19 scFv-Hinge-TM-CD28/41BB-CD3ζ wherein EGFRt is a truncated EGFR, as a safety switch (e.g., inducible cell death moiety), CD7 scFv is the scFv fragment of the heavy and light chain variable regions of an anti-CD7 antibody linked by a GS linker, and the CD19 scFv fragment is the heavy and light chain variable region of an anti-CD19 antibody linked by a GS linker. The structure of the CAR can further comprise a hinge, transmembrane regions, costimulatory signaling region of CD28 or 41BB, and/or CD3ζ intracellular domain. In the present disclosure, the nucleic acid construct of EGFRt-CD7 scFv-CD19 scFv-Hinge-TM-CD28/41BB-CD3 can be inserted into a vector (e.g., a lentiviral vector). The vector can be packaged in 293T cells. T cells can be sorted from PBMC, and after activation, TCR and PD-1 genes can be knocked out by CRISPR/CAS technology. T cells can then be infected with the vectors to express the CARs. The prepared CAR-T cells can be used to detect the infection efficiency and gene editing efficiency of CAR by flowcytometry.

In some cases, a CAR can comprise the structure of EGFRt-CD2 scFv-CD19 scFv-Hinge-TM-CD28/41BB-CD3ζ wherein EGFRt is a truncated EGFR, as a safety switch (e.g., inducible cell death moiety), CD2 scFv is the scFv fragment of the heavy and light chain variable regions of an anti-CD2 antibody linked by a GS linker, and the CD19 scFv fragment is the heavy and light chain variable region of an anti-CD19 antibody linked by a GS linker. The structure of the CAR can further comprise a hinge, transmembrane regions, costimulatory signaling region of CD28 or 41BB, and/or CD3ζ intracellular domain. In the present disclosure, the nucleic acid construct of EGFRt-CD2 scFv-CD19 scFv-Hinge-TM-CD28/41BB-CD3 can be inserted into a vector (e.g., a lentiviral vector). The vector can be packaged in 293T cells. T cells can be sorted from PBMC, and after activation, TCR and PD-1 genes can be knocked out by CRISPR/CAS technology. T cells can then be infected with the vectors to express the CARs. The prepared CAR-T cells can be used to detect the infection efficiency and gene editing efficiency of CAR by flowcytometry.

In some cases, a CAR can comprise the structure of EGFRt-CD3 scFv-CD19 scFv-Hinge-TM-CD28/41BB-CD3ζ wherein EGFRt is a truncated EGFR, as a safety switch (e.g., inducible cell death moiety), CD3 scFv is the scFv fragment of the heavy and light chain variable regions of an anti-CD3 antibody linked by a GS linker, and the CD19 scFv fragment is the heavy and light chain variable region of an anti-CD19 antibody linked by a GS linker. The structure of the CAR can further comprise a hinge, transmembrane regions, costimulatory signaling region of CD28 or 41BB, and/or CD3ζ intracellular domain. In the present disclosure, the nucleic acid construct of EGFRt-CD3 scFv-CD19 scFv-Hinge-TM-CD28/41BB-CD3 can be inserted into a vector (e.g., a lentiviral vector). The vector can be packaged in 293T cells. T cells can be sorted from PBMC, and after activation, TCR and PD-1 genes can be knocked out by CRISPR/CAS technology. T cells can then be infected with the vectors to express the CARs. The prepared CAR-T cells can be used to detect the infection efficiency and gene editing efficiency of CAR by flowcytometry.

In some cases, a CAR can comprise the structure of EGFRt-CD5 scFv-CD19 scFv-Hinge-TM-CD28/41BB-CD3, wherein EGFRt is a truncated EGFR, as a safety switch (e.g., inducible cell death moiety), CD5 scFv is the scFv fragment of the heavy and light chain variable regions of an anti-CD5 antibody linked by a GS linker, and the CD19 scFv fragment is the heavy and light chain variable region of an anti-CD19 antibody linked by a GS linker. The structure of the CAR can further comprise a hinge, transmembrane regions, costimulatory signaling region of CD28 or 41BB, and/or CD3ζ intracellular domain. In the present disclosure, the nucleic acid construct of EGFRt-CD5 scFv-CD19 scFv-Hinge-TM-CD28/41BB-CD3 can be inserted into a vector (e.g., a lentiviral vector). The vector can be packaged in 293T cells. T cells can be sorted from PBMC, and after activation, TCR and PD-1 genes can be knocked out by CRISPR/CAS technology. T cells can then be infected with the vectors to express the CARs. The prepared CAR-T cells can be used to detect the infection efficiency and gene editing efficiency of CAR by flowcytometry.

In some cases, a CAR comprising two antigen binding domains arranged in a tandem form. In some embodiments, the first antigen binding domain and the second antigen binding domain is arranged, from amino terminus to carboxyl terminus, as: (i) VL2-VH2-VL1-VH1; (ii) VL2-VH2-VH1-VL1; (iii) VL1-VH1-VL2-VH2; (iv) VL1-VH1-VH2-VL2; (v) VH2-VL2-VL1-VH1; (vi) VH2-VL2-VH1-VL1; (vii) VH1-VL1-VL2-VH2; or (viii) VH1-VL1-VH2-VL2, wherein VH1 is heavy chain variable domain of the first antigen binding domain, VL1 is light chain variable light domain of the first antigen binding domain, VH2 is heavy chain variable domain of the second antigen binding domain, and VL2 is light chain variable domain of the second antigen binding domain. For example, the CAR can have a structure represented by the following formula IV or IV′: L3-scFv1-R-scFv2-H3-TM3-C3-CD3ζ (IV); L3-scFv2-R-scFv1-H3-TM3-C3-CD3ζ (IV′), wherein each “-” is independently a linker peptide or peptide bond; L3 is an optional signal peptide sequence; scFv1 is an antigen binding domain that targets CD19; R is a rigid or flexible joint; scFv2 is an antigen binding domain that targets T cell and NK cell consensus markers such as CD2, CD3, CD5 or CD7; H3 is an optional hinge region; TM3 is a transmembrane domain; C3 is a costimulatory domain; CD3ζ is a cytoplasmic signaling sequence derived from CD3ζ.

In some cases, a CAR comprising two antigen binding domains arranged in a tandem form. In some embodiments, the first antigen binding domain and the second antigen binding domain is arranged, from amino terminus to carboxyl terminus, as: (i) VL2-VH2-VL1-VH1; (ii) VL2-VH2-VH1-VL1; (iii) VL1-VH1-VL2-VH2; (iv) VL1-VH1-VH2-VL2; (v) VH2-VL2-VL1-VH1; (vi) VH2-VL2-VH1-VL1; (vii) VH1-VL1-VL2-VH2; or (viii) VH1-VL1-VH2-VL2, wherein VH1 is heavy chain variable domain of the first antigen binding domain, VL1 is light chain variable light domain of the first antigen binding domain, VH2 is heavy chain variable domain of the second antigen binding domain, and VL2 is light chain variable domain of the second antigen binding domain. For example, the CAR can have a structure represented by the following formula IV or IV′: L3-scFv1-R-scFv2-H3-TM3-C3-CD3ζ (IV); L3-scFv2-R-scFv1-H3-TM3-C3-CD3ζ (IV′), wherein each “-” is independently a linker peptide or peptide bond; L3 is an optional signal peptide sequence; scFv1 is an antigen binding domain that targets CD19; R is a rigid or flexible joint; scFv2 is an antigen binding domain that targets CD7; H3 is an optional hinge region; TM3 is a transmembrane domain; C3 is a costimulatory domain; CD3ζ is a cytoplasmic signaling sequence derived from CD3ζ.

In some cases, a CAR comprising two antigen binding domains arranged in a tandem form. In some embodiments, the first antigen binding domain and the second antigen binding domain is arranged, from amino terminus to carboxyl terminus, as: (i) VL2-VH2-VL1-VH1; (ii) VL2-VH2-VH1-VL1; (iii) VL1-VH1-VL2-VH2; (iv) VL1-VH1-VH2-VL2; (v) VH2-VL2-VL1-VH1; (vi) VH2-VL2-VH1-VL1; (vii) VH1-VL1-VL2-VH2; or (viii) VH1-VL1-VH2-VL2, wherein VH1 is heavy chain variable domain of the first antigen binding domain, VL1 is light chain variable light domain of the first antigen binding domain, VH2 is heavy chain variable domain of the second antigen binding domain, and VL2 is light chain variable domain of the second antigen binding domain. For example, the CAR can have a structure represented by the following formula IV or IV′: L3-scFv1-R-scFv2-H3-TM3-C3-CD3ζ (IV); L3-scFv2-R-scFv1-H3-TM3-C3-CD3ζ (IV′), wherein each “-” is independently a linker peptide or peptide bond; L3 is an optional signal peptide sequence; scFv1 is an antigen binding domain that targets CD19; R is a rigid or flexible joint; scFv2 is an antigen binding domain that targets CD2; H3 is an optional hinge region; TM3 is a transmembrane domain; C3 is a costimulatory domain; CD3ζ is a cytoplasmic signaling sequence derived from CD3ζ.

In some cases, a CAR comprising two antigen binding domains arranged in a tandem form. In some embodiments, the first antigen binding domain and the second antigen binding domain is arranged, from amino terminus to carboxyl terminus, as: (i) VL2-VH2-VL1-VH1; (ii) VL2-VH2-VH1-VL1; (iii) VL1-VH1-VL2-VH2; (iv) VL1-VH1-VH2-VL2; (v) VH2-VL2-VL1-VH1; (vi) VH2-VL2-VH1-VL1; (vii) VH1-VL1-VL2-VH2; or (viii) VH1-VL1-VH2-VL2, wherein VH1 is heavy chain variable domain of the first antigen binding domain, VL1 is light chain variable light domain of the first antigen binding domain, VH2 is heavy chain variable domain of the second antigen binding domain, and VL2 is light chain variable domain of the second antigen binding domain. For example, the CAR can have a structure represented by the following formula IV or IV′: L3-scFv1-R-scFv2-H3-TM3-C3-CD3ζ (IV); L3-scFv2-R-scFv1-H3-TM3-C3-CD3ζ (IV′), wherein each “-” is independently a linker peptide or peptide bond; L3 is an optional signal peptide sequence; scFv1 is an antigen binding domain that targets CD19; R is a rigid or flexible joint; scFv2 is an antigen binding domain that targets CD3; H3 is an optional hinge region; TM3 is a transmembrane domain; C3 is a costimulatory domain; CD3ζ is a cytoplasmic signaling sequence derived from CD3ζ.

In some cases, a CAR comprising two antigen binding domains arranged in a tandem form. In some embodiments, the first antigen binding domain and the second antigen binding domain is arranged, from amino terminus to carboxyl terminus, as: (i) VL2-VH2-VL1-VH1; (ii) VL2-VH2-VH1-VL1; (iii) VL1-VH1-VL2-VH2; (iv) VL1-VH1-VH2-VL2; (v) VH2-VL2-VL1-VH1; (vi) VH2-VL2-VH1-VL1; (vii) VH1-VL1-VL2-VH2; or (viii) VH1-VL1-VH2-VL2, wherein VH1 is heavy chain variable domain of the first antigen binding domain, VL1 is light chain variable light domain of the first antigen binding domain, VH2 is heavy chain variable domain of the second antigen binding domain, and VL2 is light chain variable domain of the second antigen binding domain. For example, the CAR can have a structure represented by the following formula IV or IV′: L3-scFv1-R-scFv2-H3-TM3-C3-CD3ζ (IV); L3-scFv2-R-scFv1-H3-TM3-C3-CD3ζ (IV′), wherein each “-” is independently a linker peptide or peptide bond; L3 is an optional signal peptide sequence; scFv1 is an antigen binding domain that targets CD19; R is a rigid or flexible joint; scFv2 is an antigen binding domain that targets CD5; H3 is an optional hinge region; TM3 is a transmembrane domain; C3 is a costimulatory domain; CD3ζ is a cytoplasmic signaling sequence derived from CD3ζ.

In some cases, a CAR comprising two antigen binding domains arranged in a loop form. In some cases, the first antigen binding domain and the second antigen binding domain is arranged, from amino terminus to carboxyl terminus, as: (i) VL2-VH1-VL1-VH2; (ii) VH2-VL1-VH1-VL2; (iii) VL1-VH2-VL2-VH1; (iv) VH1-VL2-VH2-VL1; (v) VL2-VL1-VH1-VH2; (vi) VH2-VH1-VL1-VL2; (vii) VL1-VL2-VH2-VH1; or (viii) VH1-VH2-VL2-VL1, wherein VH1 is heavy chain variable domain of the first antigen binding domain, VL1 is light chain variable light domain of the first antigen binding domain, VH2 is heavy chain variable domain of the second antigen binding domain, and VL2 is light chain variable domain of the second antigen binding domain. For example, the CAR can have the following formula VI, VI′, VI″ or VI′″ structure: L8-VL1-VH2-I-VL2-VH1-H8-TM8-C8-CD3ζ (VI); L8-VH1-VL2-I-VH2-VL1-H8-TM8-C8-CD3ζ (VI′); L8-VL2-VH1-I-VL1-VH2-H8-TM8-C8-CD3ζ (VI″); L8-VH2-VL1-I-VH1-VL2-H8-TM8-C8-CD3ζ (VI′″), wherein each “-” is independently a linker peptide or peptide bond; L8 is an optional signal peptide sequence; VH1 is an anti-CD19 antibody heavy chain variable region, and VL1 is an anti-CD19 antibody light chain variable region; VH2 is an anti-T cell and NK cell consensus marker (such as CD7, CD5, CD3 or CD2) antibody heavy chain variable region; and VL2 is an anti-T cell and NK cell consensus marker (such as CD7, CD3, CD5 or CD2) antibody light chain variable region; I is a flexible joint; H8 is an optional hinge region; TM8 is a transmembrane domain; C8 is a costimulatory domain; CD3ζ is a cytoplasmic signaling sequence derived from CD3ζ.

In some cases, the CAR can have the following formula VI, VI′, VI″ or VI′″ structure: L8-VL1-VH2-I-VL2-VH1-H8-TM8-C8-CD3ζ (VI); L8-VH1-VL2-I-VH2-VL1-H8-TM8-C8-CD3ζ (VI′); L8-VL2-VH1-I-VL1-VH2-H8-TM8-C8-CD3ζ (VI″); L8-VH2-VL1-I-VH1-VL2-H8-TM8-C8-CD3ζ (VI′″), wherein each “-” is independently a linker peptide or peptide bond; L8 is an optional signal peptide sequence; VH1 is an anti-CD19 antibody heavy chain variable region, and VL1 is an anti-CD19 antibody light chain variable region; VH2 is an anti-CD7 antibody heavy chain variable region; and VL2 is an anti-CD7 antibody light chain variable region; I is a flexible joint; H8 is an optional hinge region; TM8 is a transmembrane domain; C8 is a costimulatory domain; CD3ζ is a cytoplasmic signaling sequence derived from CD3.

In some cases, the CAR can have the following formula VI, VI′, VI″ or VI′″ structure: L8-VL1-VH2-I-VL2-VH1-H8-TM8-C8-CD3ζ (VI); L8-VH1-VL2-I-VH2-VL1-H8-TM8-C8-CD3ζ (VI′); L8-VL2-VH1-I-VL1-VH2-H8-TM8-C8-CD3ζ (VI″); L8-VH2-VL1-I-VH1-VL2-H8-TM8-C8-CD3ζ (VI′″), wherein each “-” is independently a linker peptide or peptide bond; L8 is an optional signal peptide sequence; VH1 is an anti-CD19 antibody heavy chain variable region, and VL1 is an anti-CD19 antibody light chain variable region; VH2 is an anti-CD2 antibody heavy chain variable region; and VL2 is an anti-CD2 antibody light chain variable region; I is a flexible joint; H8 is an optional hinge region; TM8 is a transmembrane domain; C8 is a costimulatory domain; CD3 is a cytoplasmic signaling sequence derived from CD3.

In some cases, the CAR can have the following formula VI, VI′, VI″ or VI′″ structure: L8-VL1-VH2-I-VL2-VH1-H8-TM8-C8-CD3ζ (VI); L8-VH1-VL2-I-VH2-VL1-H8-TM8-C8-CD3ζ (VI′); L8-VL2-VH1-I-VL1-VH2-H8-TM8-C8-CD3ζ (VI″); L8-VH2-VL1-I-VH1-VL2-H8-TM8-C8-CD3ζ (VI′″), wherein each “-” is independently a linker peptide or peptide bond; L8 is an optional signal peptide sequence; VH1 is an anti-CD19 antibody heavy chain variable region, and VL1 is an anti-CD19 antibody light chain variable region; VH2 is an anti-CD3 antibody heavy chain variable region; and VL2 is an anti-CD3 antibody light chain variable region; I is a flexible joint; H8 is an optional hinge region; TM8 is a transmembrane domain; C8 is a costimulatory domain; CD3 is a cytoplasmic signaling sequence derived from CD3.

In some cases, the CAR can have the following formula VI, VI′, VI″ or VI′″ structure: L8-VL1-VH2-I-VL2-VH1-H8-TM8-C8-CD3ζ (VI); L8-VH1-VL2-I-VH2-VL1-H8-TM8-C8-CD3ζ (VI′); L8-VL2-VH1-I-VL1-VH2-H8-TM8-C8-CD3ζ (VI″); L8-VH2-VL1-I-VH1-VL2-H8-TM8-C8-CD3ζ (VI′″), wherein each “-” is independently a linker peptide or peptide bond; L8 is an optional signal peptide sequence; VH1 is an anti-CD19 antibody heavy chain variable region, and VL1 is an anti-CD19 antibody light chain variable region; VH2 is an anti-CD5 antibody heavy chain variable region; and VL2 is an anti-CD5 antibody light chain variable region; I is a flexible joint; H8 is an optional hinge region; TM8 is a transmembrane domain; C8 is a costimulatory domain; CD3ζ is a cytoplasmic signaling sequence derived from CD3.

In some cases, a CAR comprising two antigen binding domains are arranged in a parallel form. The parallel form can comprise a full construct of a first CAR having a first antigen binding domain and a full construct of a second CAR having a second antigen binding domain. Accordingly, the immune cell disclosed herein may comprise a first antigen binding domain that binds to CD19 as disclosed herein, a second antigen binding domain binds to CD7 as disclosed herein, a transmembrane domain and a second transmembrane domain as disclosed herein and an intracellular signaling domain and a second intracellular signaling domain as disclosed herein. In some cases, the first antigen binding domain is linked to an intracellular signaling domain via a transmembrane domain. In some cases, the second antigen binding domain is linked to an intracellular signaling domain via a transmembrane domain. In some cases, the first antigen binding domain is linked to an intracellular signaling domain via a transmembrane domain and the second antigen binding domain is linked to another intracellular signaling domain via another transmembrane domain. In some cases, the first antigen binding domain is linked to the intracellular signaling domain via the transmembrane domain and the second antigen binding domain is linked to the second intracellular signaling domain via the second transmembrane domain. In some cases, the first antigen binding domain is linked to one intracellular signaling domain via one transmembrane domain and the other antigen binding domain is linked to the second intracellular signaling domain via the other transmembrane domain. An example of parallel form can be tEGFR-CD19 scFv-CD28-CD3ζ-CD7 scFv-41BB-CD3ζ. The tEGFR shown here can function as a safety switch, which can be replaced by other safety switches as described in the present disclosure. As described herein, CD19 scFv and CD7 scFv are two examples of antigen binding domains, which may be replaced with various antigen binding domains as described in the present disclosure. In some cases, the parallel form can be tEGFR-CD19 scFv-CD28-CD3ζ-CD5 scFv-41BB-CD3ζ. In some cases, the parallel form can be tEGFR-CD19 scFv-CD28-CD3ζ-CD3 scFv-41BB-CD3ζ. In some cases, the parallel form can be tEGFR-CD19 scFv-CD28-CD3ζ-CD2 scFv-41BB-CD3. CD28 can be an example of transmembrane domain and can be replaced with other transmembrane domains described herein. 41BB can be an example of co-stimulatory domain and can be replaced with other co-stimulatory domains described herein. In some cases, a linker is used to link the first CAR and the second CAR. The linker can be a cleavable linker. The cleavable linker can be self-cleaving peptide such as 2A self-cleaving peptide.

Also contemplated in the present disclosure is a nucleic acid molecule encoding a CAR or a bispecific CAR. The nucleic acid can comprise a first sequence encoding a chimeric antigen receptor (CAR), wherein the CAR can comprise a binding moiety, which binding moiety comprises (i) a first antigen binding domain, which first antigen binding domain suppresses or reduces a subject's immune response toward the engineered immune cell when administered into the subject linked to (ii) a second antigen binding domain capable of binding to CD19, and wherein each CAR of the one or more CARs can further comprise a transmembrane domain and an intracellular signaling domain. The first antigen binding domain can target an immune cell antigen selected from the group consisting of CD2, CD3, CD5, and CD7.

In some cases, the nucleic acid can comprise a first sequence encoding a chimeric antigen receptor (CAR), wherein the CAR can comprise a binding moiety, which binding moiety comprises (i) a first antigen binding domain capable of binding to CD7 linked to (ii) a second antigen binding domain capable of binding to CD19, and wherein the CAR can further comprise a transmembrane domain and an intracellular signaling domain. In some cases, the nucleic acid can comprise a first sequence encoding a chimeric antigen receptor (CAR), wherein the CAR can comprise a binding moiety, which binding moiety comprises (i) a first antigen binding domain capable of binding to CD2 linked to (ii) a second antigen binding domain capable of binding to CD19, and wherein the CAR can further comprise a transmembrane domain and an intracellular signaling domain. In some cases, the nucleic acid can comprise a first sequence encoding a chimeric antigen receptor (CAR), wherein the CAR can comprise a binding moiety, which binding moiety comprises (i) a first antigen binding domain capable of binding to CD3 linked to (ii) a second antigen binding domain capable of binding to CD19, and wherein the CAR can further comprise a transmembrane domain and an intracellular signaling domain. In some cases, the nucleic acid can comprise a first sequence encoding a chimeric antigen receptor (CAR), wherein the CAR can comprise a binding moiety, which binding moiety comprises (i) a first antigen binding domain capable of binding to CD5 linked to (ii) a second antigen binding domain capable of binding to CD19, and wherein the CAR can further comprise a transmembrane domain and an intracellular signaling domain. The nucleic acid molecule can further comprise a second sequence encoding an enhancer moiety, which enhancer moiety can enhance one or more activities of the CAR when expressed in a cell. The enhancer moiety can be selected from the group consisting of IL-2, IL-3, IL-4, IL-6, IL-7, IL-8, IL-10, IL-11, IL-12, IL-15, IL-17, IL-18, IL-21, IL-23, PD-1, PD-L1, CD122, CSF1R, CTAL-4, TIM-3, CCL21, CCL19, TGFR beta, receptors for the same, functional fragments thereof, functional variants thereof, and combinations thereof. The nucleic acid molecule can further comprise a second sequence encoding an inducible cell death moiety, which inducible cell death moiety, when expressed in a cell, can effect death of the cell upon contacting the inducible cell death moiety with a cell death activator. The inducible cell death moiety can be selected from the group consisting of rapaCasp9, iCasp9, HSV-TK, CD20, ΔCD20, mTMPK, ΔCD19, RQR8, and EGFRt.

The nuclei acid molecule can further comprise a third sequence flanked by the first sequence and the second sequence, wherein the third sequence can encode a cleavable linker. The cleavable linker can be a self-cleaving peptide.

The nucleic acid molecule can further comprise a regulatory sequence regulating expression of the first sequence and/or the second sequence.

Also contemplated in the present disclosure is a kit comprising the nucleic acid molecule described herein.

In some cases, the nucleic acid encoding the CAR described herein can be delivered into an immune cell for expression of the CAR to generate an engineered cell.

### Source Cell

The present disclosure provides an engineered cell, such as an engineered immune cell. The engineered immune cell can be prepared from a cell (e.g., an immune cell) isolated from a sample obtained from a subject. The engineered immune cell can be prepared from a cell line cell. The immune cell used to prepare the engineered immune cell can be a T cell, a B cell, a natural killer (NK) cell or a macrophage. The immune cell used to prepare the engineered immune cell can be an innate lymphocyte (ILC).

The immune cell used to prepare the engineered immune cell can be a stem cell. The stem cell can be a hematopoietic stem cell (HSC) or an induced pluripotent stem cell (iPSC).

The immune cell may comprise a T-cell receptor (TCR). The TCR can be endogenous TCR of the immune cell. In some cases, the endogenous TCR can be inactivated. For example, a gene encoding a subunit of the TCR can be inactivated. For example, the immune cell can be an alpha beta T cells with impaired TCRs such that the immune cells can avoid GVHD. For another example, the function of the endogenous TCR can be inhibited by an inhibitor such as TCR-derived peptides, peptides derived from amino acid sequences of fusion and other protein regions of various viruses, antibodies and small molecule inhibitors. The viruses from which the TCR inhibiting peptides can be derived from include, but are not limited to, severe acute respiratory syndrome coronavirus (SARS-CoV), herpesvirus saimiri (HVS), human herpesvirus 6 (HHV-6), Lassa virus (LASV), lymphocytic choriomeningitis virus (LCMV), Mopeia virus (MOPV), Tacaribe virus (TACV), Friend murine leukemia virus (MLV); human T lymphotropic virus type 1 (HTLV-1,); herpesvirus ateles (HVA); Marburg virus (MARV); Sudan Ebola virus (SEBOV); and Zaire Ebola virus (ZEBOV).

In some cases, the immune cells can be T cells containing TCRs that may not cause GVHD responses. For example, the immune cell can be an alpha beta T cell with TCRs that can recognize specific antigens such as viral specific antigen, tumor-associated antigens (TAAs) or tumor-specific antigens (TSAs). For another example, the immune cell can be a gamma delta T cell or a natural killer T (NKT) cell. For another example, the immune cell can be induced pluripotent stem cells produced from antigen-specific T cells (e.g., antigen-specific cytotoxic T cells). The immune cell can be cord-blood T cells.

The immune cell may comprise a cell surface marker. The cell surface marker can be an immune cell antigen. The gene encoding the immune cell antigen of the immune cell used for preparing the engineered immune cell can be inactivated. Examples of immune cell antigens include, but are not limited to, CD2, CD3, CD4, CD5, CD7, CD8, CD16a, CD16b, CD25, CD27, CD28, CD30, CD38, CD45, CD48, CD50, CD52, CD56, CD57, CD62L, CD69, CD94, CD100, CD102, CD122, CD127, CD132, CD137, CD160, CD161, CD178, CD218, CD226, CD244, CD159a (NKG2A), CD159c (NKG2C), NKG2E, CD279, CD314 (NKG2D), CD305, CD335 (NKP46), CD337, CD319 (CS1), TCRα, TCRβ and SLAMF7. For example, in some cases, the gene encoding CD7 of the immune cell is inactivated. In some cases, the gene encoding CD3 of the immune cell is inactivated. In some cases, the gene encoding CD137 of the immune cell is inactivated.

The immune cells can be isolated from a sample from a subject. The subject can be a healthy donor. The subject can have a condition (e.g., a disease such as cancer). The sample can be a bodily fluid or a tissue, including but not limited to, peripheral blood mononuclear cells, bone marrow, lymph node tissue, cord blood, thymus tissue, tissue from a site of infection, ascites, pleural effusion, spleen tissue, and tumors. In some cases, a sample comprises NK cells, NKT cells, T-cells or T-cell progenitor cells. For example, in some cases, the sample is an umbilical cord blood sample, a peripheral blood sample (e.g., a mononuclear cell fraction) or a sample from the subject comprising pluripotent cells. In some aspects, a sample from the subject can be cultured to generate induced pluripotent stem (iPS) cells and these cells used to produce NK cells, NKT cells or T-cells. Cell samples may be cultured directly from the subject or may be cryopreserved prior to use. In some aspects, obtaining a cell sample comprises collecting a cell sample. In other aspects, the sample is obtained by a third party. In still further aspects, a sample from a subject can be treated to purify or enrich the T-cells or T-cell progenitors in the sample. For example, the sample can be subjected to gradient purification, cell culture selection and/or cell sorting (e.g., via fluorescence-activated cell sorting (FACS)).

The immune cell can be an NK cell. The NK cells can be obtained from peripheral blood, cord-blood, or other sources described herein. The NK cells can be derived from induced pluripotent stem cells.

In some embodiments, a cell that can be utilized in a method provided herein can be positive or negative for a given factor. In some embodiments, a cell utilized in a method provided herein can be a CD3+ cell, CD3− cell, a CD5+ cell, CD5− cell, a CD7+ cell, CD7− cell, a CD14+ cell, CD14− cell, CD8+ cell, a CD8− cell, a CD103+ cell, CD103− cell, CD11b+ cell, CD11b− cell, a BDCA1+ cell, a BDCA1− cell, an L-selectin+ cell, an L-selectin− cell, a CD25+, a CD25− cell, a CD27+, a CD27− cell, a CD28+ cell, CD28− cell, a CD44+ cell, a CD44− cell, a CD56+ cell, a CD56− cell, a CD57+ cell, a CD57− cell, a CD62L+ cell, a CD62L− cell, a CD69+ cell, a CD69− cell, a CD45RO+ cell, a CD45RO− cell, a CD127+ cell, a CD127− cell, a CD132+ cell, a CD132− cell, an IL-7+ cell, an IL-7− cell, an IL-15+ cell, an IL-15− cell, a lectin-like receptor G1 positive cell, a lectin-like receptor G1 negative cell, or an differentiated or de-differentiated cell thereof. The examples of factors expressed by cells is not intended to be limiting, and a person having skill in the art will appreciate that a cell may be positive or negative for any factor known in the art. In some embodiments, a cell may be positive for two or more factors. For example, a cell may be CD4+ and CD8+. In some embodiments, a cell may be negative for two or more factors. For example, a cell may be CD25−, CD44−, and CD69−. In some embodiments, a cell may be positive for one or more factors, and negative for one or more factors. For example, a cell may be CD4+ and CD8−. In some aspects, a cellular marker provided herein can be utilized to select, enrich, or deplete a population of cells. In some aspects, enriching comprises selecting a monocyte fraction. In some aspects, enriching comprises sorting a population of immune cells from a monocyte fraction. In some embodiments, the cells may be selected for having or not having one or more given factors (e.g., cells may be separated based on the presence or absence of one or more factors). In some embodiments, the selected cells can also be transduced and/or expanded in vitro. The selected cells can be expanded in vitro prior to infusion. In some embodiments, selected cells can be transduced with a vector provided herein. It should be understood that cells used in any of the methods disclosed herein may be a mixture (e.g., two or more different cells) of any of the cells disclosed herein. For example, a method of the present disclosure may comprise cells, and the cells are a mixture of CD4+ cells and CD8+ cells. In another example, a method of the present disclosure may comprise cells, and the cells are a mixture of CD4+ cells and naïve cells. In some cases, a cell can be a stem memory TSCM cell comprised of CD45RO (−), CCR7(+), CD45RA (+), CD62L+(L-selectin), CD27+, CD28+ and IL-7Rα+, stem memory cells can also express CD95, IL-2Rβ, CXCR3, and LFA-1, and show numerous functional attributes distinctive of stem memory cells. Cells provided herein can also be central memory TCM cells comprising L-selectin and CCR7, where the central memory cells can secrete, for example, IL-2, but not IFNγ or L-4. Cells can also be effector memory TEM cells comprising L-selectin or CCR7 and produce, for example, effector cytokines such as IFNγ and IL-4. In some cases, a population of cells can be introduced to a subject. For example, a population of cells can be a combination of T cells and NK cells. In other cases, a population can be a combination of naïve cells and effector cells. A population of cells can be TILs.

The source immune cells can be T cells. The T cells can be alpha beta T cells or gamma delta T cells. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, various T cell lines may be used. In certain embodiments of the present disclosure, T cells can be obtained from a unit of blood collected from a subject using any number of techniques known to the skilled artisan, such as Ficoll™ separation. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing steps. In some embodiments of the present disclosure, the cells are washed with phosphate buffered saline (PBS). In an alternative embodiment, the wash solution lacks calcium and may lack magnesium or may lack many if not all divalent cations. Initial activation steps in the absence of calcium may lead to magnified activation. A washing step may be accomplished by methods such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca2+-free, Mg2+-free PBS, PlasmaLyte A, or other saline solution with or without buffer. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+T cells, can be further isolated by positive or negative selection techniques. For example, in one embodiment, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In some embodiments, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. In some cases, multiple rounds of selection can also be used. In certain embodiments, it may be useful to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. An example method can be cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4+, CD25+, CD62Lhi, GITR+, and FoxP3+. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related embodiment, lower concentrations of cells may be used. By significantly diluting the mixture of T cells and surface (e.g., particles such as beads), interactions between the particles and cells is minimized. This method can select for cells that express high amounts of desired antigens to be bound to the particles. For example, CD4+ T cells express higher levels of CD28 and are more efficiently captured than CD8+ T cells in dilute concentrations. In some embodiments, the concentration of cells used is 5×10⁶/ml. In other embodiments, the concentration used can be from about 1×10⁵/ml to 1×10⁶/ml, and any integer value in between. In other embodiments, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In some embodiments, cryopreserved cells are thawed and washed as described herein and allowed to rest for one hour at room temperature prior to activation using the methods of the present disclosure.

In some embodiments, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun. 73:316-321, 1991; Bierer et al., Curr. Opin. Immun. 5:763-773, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

### Engineered Immune Cell

The engineered immune cell provided herein can exhibit enhanced activity toward tumor cells, but with reduced side effects such as GVHD. The engineered immune cell can target a disease-associated antigen CD19 and at the same time suppress host immune cells. One or more endogenous genes (e.g., a gene encoding a subunit of a TCR, or a gene encoding a cell surface marker) of the engineered immune cell can be inactivated. In some cases, the engineered immune cell comprises a first CAR and a second CAR, each targeting a different antigen. In some cases, the engineered immune cell comprises a CAR having a first antigen binding domain and a second antigen binding domain.

The engineered immune cell can comprise one or more chimeric antigen receptors (CARs), and the CAR can comprise a first antigen binding domain capable of binding to an immune cell antigen and a second antigen binding domain capable of binding to a disease-associated antigen. In some embodiment, the CAR can comprise a first antigen binding domain capable of binding to CD7 and a second antigen binding domain capable of binding to CD19. In some embodiment, the CAR can comprise a first antigen binding domain capable of binding to CD2 and a second antigen binding domain capable of binding to CD19. In some embodiment, the CAR can comprise a first antigen binding domain capable of binding to CD3 and a second antigen binding domain capable of binding to CD19. In some embodiment, the CAR can comprise a first antigen binding domain capable of binding to CD5 and a second antigen binding domain capable of binding to CD19. Each CAR of the one or more CARs may further comprise a transmembrane domain and an intracellular signaling domain. The engineered immune cell can also comprise an enhancer moiety capable of enhancing one or more activities of the engineered immune cell. The endogenous T cell receptor (TCR) of the engineered immune cell can be inactivated. In some cases, the engineered immune cell can exhibit (i) enhanced degree of persistence by remaining viable in vitro for at least about 20 days while in presence of cells that are heterologous to the engineered immune cell, (ii) enhanced degree of expansion by at least about 10-fold within 15 days, or (iii) enhanced cytotoxicity against a target cell comprising the immune cell antigen such as CD2, CD3, CD5 and CD7 or the disease-associated antigen such as CD19, compared to an additional engineered immune cell comprising the one or more CARs but not the enhancer moiety. In some cases, the engineered immune cell can be characterized by exhibiting two or more of (i) enhanced degree of persistence, (ii) enhanced degree of expansion, and (iii) enhanced cytotoxicity. The (i), (ii), and/or (iii) characteristics can be measured in absence of any exogenous enhancer moiety such as exogenous cytokines.

The engineered immune cell can comprise a multi-specific CAR. In some cases, the engineered immune cell comprises a bispecific CAR targeting an immune cell antigen and a disease-associated antigen. In some cases, the engineered immune cell comprises a bispecific CAR targeting CD7 and CD19. In some cases, the engineered immune cell comprises a bispecific CAR targeting CD2 and CD19. In some cases, the engineered immune cell comprises a bispecific CAR targeting CD3 and CD19. In some cases, the engineered immune cell comprises a bispecific CAR targeting CD5 and CD19. The two antigen binding domains of the bispecific CAR can be arranged in any form as described in the present disclosure, for example, parallel form, loop form, and tandem form. For example, an engineered immune cell described herein can comprise comprising a single chimeric antigen receptor (CAR) comprising (i) a first antigen binding domain that specifically binds CD7 and (ii) a second antigen binding domain capable of binding to CD19. In another example, an engineered immune cell described herein can comprise comprising a single chimeric antigen receptor (CAR) comprising (i) a first antigen binding domain that specifically binds CD2 and (ii) a second antigen binding domain capable of binding to CD19. In another example, an engineered immune cell described herein can comprise comprising a single chimeric antigen receptor (CAR) comprising (i) a first antigen binding domain that specifically binds CD3 and (ii) a second antigen binding domain capable of binding to CD19. In another example, an engineered immune cell described herein can comprise comprising a single chimeric antigen receptor (CAR) comprising (i) a first antigen binding domain that specifically binds CD5 and (ii) a second antigen binding domain capable of binding to CD19. The CAR can further comprise a transmembrane domain and an intracellular signaling domain. In some cases, a gene encoding endogenous CD7 can be inactivated (e.g., silenced or knocked out) in the engineered immune cell. In some cases, a gene encoding endogenous CD2 can be inactivated (e.g., silenced or knocked out) in the engineered immune cell. In some cases, a gene encoding endogenous CD3 or any component in the TCR complex (e.g. TRAC, TRBC, CD3γ, CD3δ, CD3ε, or CD3ζ) can be inactivated (e.g., silenced or knocked out) in the engineered immune cell. In some cases, a gene encoding endogenous CD5 can be inactivated (e.g., silenced or knocked out) in the engineered immune cell.

The enhancer moiety can be configured to constitutively enhance the one or more activities of the engineered immune cell. The enhancer moiety can be configured to constitutively upregulate one or more intracellular signaling pathways of the engineered immune cell. The one or more intracellular signaling pathways can be one or more cytokine signaling pathways. The enhancer moiety can be self-activating through self-oligomerizing. The enhancer moiety can be self-activating through self-dimerizing.

The enhancer moiety can be a cytokine or a cytokine receptor. The enhancer moiety can be selected from the group consisting of IL-2, IL-3, IL-4, IL-6, IL-7, IL-8, IL-10, IL-11, IL-12, IL-15, IL-17, IL-18, IL-21, IL-23, PD-1, PD-L1, CD122, CSF1R, CTAL-4, TIM-3, CCL21, CCL19, TGFR beta, receptors for the same, functional fragments thereof, functional variants thereof, and combinations thereof.

In some cases, a gene encoding a subunit of the endogenous TCR of the engineered immune cell can be inactivated such that the endogenous TCR is inactivated. The gene encoding the subunit can be TCRα, TCRβ, CD3ε, CD3δ, CD3γ, or CD3ζ.

The engineered immune cell can further comprise an inducible cell death moiety, which inducible cell death moiety can effect suicide of the engineered immune cell upon contact with a cell death activator. The inducible cell death moiety can be selected from the group consisting of rapaCasp9, iCasp9, HSV-TK, CD20, ΔCD20, mTMPK, ΔCD19, RQR8, and EGFRt. In some cases, the inducible cell death moiety is EGFRt, and the cell death activator is an antibody or an antigen binding fragment thereof that binds EGFRt. In some cases, the inducible cell death moiety is HSV-TK, and the cell death activator is GCV. In some cases, the inducible cell death moiety is iCasp9, and the cell death activator is AP1903. The cell death activator can comprise a nucleic acid, a polynucleotide, an amino acid, a polypeptide, lipid, a carbohydrate, a small molecule, an enzyme, a ribosome, a proteasome, a variant thereof, or any combination thereof.

The expression of one or more endogenous human leukocyte antigen (HLA) genes of the engineered immune cell may remain intact. In some cases, the expression of endogenous HLA-I and/or HLA-II genes of the engineered immune cell may remain intact. The expression of endogenous HLA-A, HLA-B, HLA-C, HLA-E, HLA-F HLA-G, HLA-DRA, HLA-DRB, HLA-DQA and/or HLA-DQB genes of the engineered immune cell may remain intact.

The expression of one or more endogenous HLA genes of the engineered immune cell can be upregulated. In some cases, the expressions of endogenous HLA-A, HLA-B, HLA-C, HLA-E, HLA-F, HLA-G, HLA-DRA, HLA-DRB, HLA-DQA and/or HLA-DQB genes of the engineered immune cell are upregulated.

The expression of one or more endogenous HLA genes of the engineered immune cell may be suppressed, knocked out or partially knocked out. For example, HLA-I, HLA-II or both can be suppressed or knocked out. In some cases, the expressions of endogenous HLA-A, HLA-B, HLA-C, HLA-E, HLA-F, HLA-G, HLA-DRA, HLA-DRB, HLA-DQA and/or HLA-DQB genes of the engineered immune cell are suppressed or knocked out. In some cases, HLA-I, HLA-II, or both can be partially knocked out. In some cases, the expressions of endogenous HLA-A, HLA-B, HLA-C, HLA-E, HLA-F, HLA-G, HLA-DRA, HLA-DRB, HLA-DQA and/or HLA-DQB genes of the engineered immune cell are partially knockout. In some cases, HLA-I/II can be partially knockout. In some cases, an endogenous HLA can be knocked out to reduce T cell killing activity but keep anti-NK killer function. For example, HLA-A/B can be knocked out while keeping HLA-C/E in the engineered immune cell. In some cases, HLA-A, HLA-B, or both is knocked out. In some cases, HLA-C, HLA-E, or both remains intact. In some cases, a killer/phagocyte inhibitor of the engineered immune cell can be overexpressed. In some other cases, an endogenous HLA can be knocked out with co-expression of killer/phagocyte inhibitor(s). For example, HLA-I, HLA-II, or both can be knocked out with co-expression of killer/phagocyte inhibitors. The killer/phagocyte inhibitor may suppress immune response toward the engineered immune cell. The killer/phagocyte inhibitors include, but are not limited to, CD47, CD24, FASL, PDL1, or functional domains thereof.

The first antigen binding domain or the second antigen binding domain can be an antibody or fragment thereof, for example, a scFv or a single domain antibody.

A gene encoding an endogenous surface marker of the engineered immune cell can be inactivated, wherein the endogenous surface marker is capable of binding to the first antigen binding domain when expressed. The endogenous surface marker can be the antigen that CAR targets. In various embodiments, when a CAR of the engineered immune cell targets an antigen that is endogenously expressed by the engineered immune cell, the endogenous antigen or the gene encoding such antigen may be inactivated (e.g., disrupted, inhibited, silenced or knocked out). Various gene editing methods described herein can be used. The endogenous surface marker can be, for example, CD2, CD3, CD5, or CD7. In some embodiments, the endogenous surface marker is CD7. In some embodiments, the endogenous surface marker is CD2. In some embodiments, the endogenous surface marker is CD3. In some embodiments, the endogenous surface marker is CD5.

The engineered immune cell provided herein can comprise a chimeric polypeptide comprising (i) an enhancer moiety capable of enhancing one or more activities of the engineered immune cell, and (ii) an inducible cell death moiety capable of effecting death of the engineered immune cell upon contacting the chimeric polypeptide with a cell death activator, wherein the enhancer moiety is linked to the inducible cell death moiety. In some cases, the enhancer moiety and the inducible moiety may be linked by a linker. The linker can be a cleavable linker, for example, a self-cleaving peptide. In some embodiments, the CPR may comprises a binding moiety, wherein the binding moiety comprises (i) a first antigen binding domain, which first antigen binding domain suppresses or reduces a subject's immune response toward the engineered immune cell when administered into the subject and (ii) a second antigen binding domain capable of binding to disease-associated antigen. In some cases, the CPR may comprise a binding moiety, wherein the binding moiety comprises (i) a first antigen binding domain capable of binding to CD7 and (ii) a second antigen binding domain capable of binding to CD19. In some cases, the CPR may comprise a binding moiety, wherein the binding moiety comprises (i) a first antigen binding domain capable of binding to CD2 and (ii) a second antigen binding domain capable of binding to CD19. In some cases, the CPR may comprise a binding moiety, wherein the binding moiety comprises (i) a first antigen binding domain capable of binding to CD3 and (ii) a second antigen binding domain capable of binding to CD19. In some cases, the CPR may comprise a binding moiety, wherein the binding moiety comprises (i) a first antigen binding domain capable of binding to CD5 and (ii) a second antigen binding domain capable of binding to CD19. An individual CPR of the one or more CPRs can comprise (i) the first antigen binding domain, (ii) the second antigen binding domain, or (iii) both the first antigen binding domain and the second antigen binding domain. A CPR of the one or more CPRs can further comprise a transmembrane domain and an intracellular signaling domain.

In some cases, the one or more CPRs in the engineered immune cell are one or more chimeric antigen receptors (CARs) or engineered T cell receptors (TCRs). In some cases, the engineered immune cells comprise both CARs and engineered TCRs. The engineered TCR can be a TCR fusion protein. For example, the TCR fusion protein can comprise a heterologous antigen binding domain fused to one or more subunits of a TCR complex. In some cases, the TCR fusion protein can comprise a TCR subunit comprising at least a portion of a TCR extracellular domain and a TCR intracellular domain; and an antibody domain comprising an antigen binding domain, where the TCR subunit and the antibody domain are linked. The TCR fusion protein can incorporate into a TCR complex when expressed in a T cell. In some cases, the TCR fusion protein can further comprise a TCR transmembrane domain. The TCR extracellular domain, the TCR intracellular domain, or the TCR transmembrane domain can be derived from TCR alpha chain, TCR beta chain, TCR gamma chain, TCR delta chain, CD3 epsilon, CD3 gamma, CD3 delta or CD3 zeta. In some cases, an endogenous TCR of the engineered immune cell comprising an engineered TCR is inactivated. In some cases, the engineered immune cell comprising inactivated endogenous TCR may not cause GVHD. For example, a gene encoding an endogenous TCR subunit can be inactivated. For another example, a gene encoding an endogenous TCR subunit may be mutated such that an endogenous TCR may not be formed.

The engineered immune cell provided herein can comprise one or more CARs comprising (i) an enhancer moiety capable of enhancing one or more activities of the engineered immune cell, and (ii) an inducible cell death moiety capable of effecting death of the engineered immune cell upon contacting the chimeric polypeptide with a cell death activator. In some cases, the enhancer moiety is linked to the inducible cell death moiety. The one or more chimeric antigen receptors (CARs) can comprise a binding moiety. The binding moiety can comprise (i) a first antigen binding domain, which first antigen binding domain suppresses or reduces a subject's immune response toward the engineered immune cell when administered into the subject and (ii) a second antigen binding domain capable of binding to a disease-associated antigen. In some cases, the binding moiety can comprise (i) a first antigen binding domain capable of binding to CD7 and (ii) a second antigen binding domain capable of binding to CD19. In some cases, the binding moiety can comprise (i) a first antigen binding domain capable of binding to CD2 and (ii) a second antigen binding domain capable of binding to CD19. In some cases, the binding moiety can comprise (i) a first antigen binding domain capable of binding to CD3 and (ii) a second antigen binding domain capable of binding to CD19. In some cases, the binding moiety can comprise (i) a first antigen binding domain capable of binding to CD5 and (ii) a second antigen binding domain capable of binding to CD19. In some cases, an individual CAR of the one or more CARs comprises (i) the first antigen binding domain or (ii) the second antigen binding domain. In some cases, an individual CAR of the one or more CARs comprises both the first antigen binding domain and the second antigen binding domain. In some cases, each CAR of the one or more CARs further comprises a transmembrane domain and an intracellular signaling domain.

In some cases, endogenous T cell receptors (TCRs) of the engineered immune cell is inactivated. Various methods can be used to inactivate endogenous TCRs. For example, a gene encoding a subunit of the endogenous TCR can be inactivated such that the endogenous TCR is inactivated. The gene encoding the subunit can be TCRα, TCRβ, CD3ε, CD3δ, CD3γ, or CD3ζ.

The chimeric polypeptide may or may not comprise any self-cleaving peptide flanked by the enhancer moiety and the inducible cell death moiety. The enhancer moiety can be configured to constitutively enhance the one or more activities of the engineered immune cell. The enhancer moiety can be configured to constitutively upregulate one or more intracellular signaling pathways of the engineered immune cell. The one or more intracellular signaling pathways can be one or more cytokine signaling pathways. The enhancer moiety can be self-activating through self-oligomerizing. The enhancer moiety can be self-activating through self-dimerizing.

The chimeric polypeptide described herein can be a secreted protein. The chimeric polypeptide can be an intracellular protein. The chimeric polypeptide can be a transmembrane protein. The enhancer moiety or the inducible cell death moiety can be contained in an ectodomain of the transmembrane protein. The enhancer moiety or the inducible cell death moiety is contained in an endodomain of the transmembrane protein. The enhancer moiety can be contained in an endodomain of the transmembrane protein and the inducible cell death moiety can be contained in an ectodomain of the transmembrane protein. The enhancer moiety can be contained in an ectodomain of the transmembrane protein, and the inducible cell death moiety can be contained in an endodomain of the transmembrane protein. The enhancer moiety can be a cytokine or a cytokine receptor. For example, the enhancer moiety can be selected from the group consisting of IL-2, IL-3, IL-4, IL-6, IL-7, IL-8, IL-10, IL-11, IL-12, IL-15, IL-17, IL-18, IL-21, IL-23, PD-1, PD-L1, CD122, CSF1R, CTAL-4, TIM-3, CCL21, CCL19, TGFR beta, receptors for the same, functional fragments thereof, functional variants thereof, and combinations thereof. The inducible cell death moiety can be selected from the group consisting of rapaCasp9, iCasp9, HSV-TK, CD20, ΔCD20, mTMPK, ΔCD19, RQR8, and EGFRt. In some cases, the inducible cell death moiety is EGFRt, and the cell death activator is an antibody or an antigen binding fragment thereof that binds EGFRt. In some cases, the inducible cell death moiety is HSV-TK, and the cell death activator is GCV. In some cases, the inducible cell death moiety is iCasp9, and the cell death activator is AP1903. The cell death activator may comprise a nucleic acid, a polynucleotide, an amino acid, a polypeptide, lipid, a carbohydrate, a small molecule, an enzyme, a ribosome, a proteasome, a variant thereof, or any combination thereof.

The engineered immune cell can be a CAR-T cell. The CAR-T cell can express a CAR targeting CD19 and CD7. The CAR-T cell can express a CAR targeting CD19 and CD2. The CAR-T cell can express a CAR targeting CD19 and CD3. The CAR-T cell can express a CAR targeting CD19 and CD5. The expression of endogenous CD7, CD2, CD3 or CD5 gene can be silenced in the CAR-T cell. The CAR can be a single CAR targeting both CD19 and CD7. The CAR can be a single CAR targeting both CD19 and CD2. The CAR can be a single CAR targeting both CD19 and CD5. The CAR can comprise a first CAR targeting CD19 and a second CAR targeting CD7. The CAR can comprise a first CAR targeting CD19 and a second CAR targeting CD2. The CAR can comprise a first CAR targeting CD19 and a second CAR targeting CD7. The CAR can comprise a first CAR targeting CD19 and a second CAR targeting CD3. The CAR can comprise a first CAR targeting CD19 and a second CAR targeting CD5. The CAR-T cell can have one or more of the following characteristics: (a) expression of PD-1 gene is silenced in the CAR-T cell; (b) expression of TCR gene is silenced in the CAR-T cell; (c) the CAR-T cell expresses an exogenous cellular suicide element (e.g., inducible cell death moiety).

The engineered immune cell can express a CAR and/or an exogenous TCR, and the CAR and/or exogenous TCR target CD19 and CD7. The engineered immune cell can express a CAR and/or an exogenous TCR, and the CAR and/or exogenous TCR target CD19 and CD2. The engineered immune cell can express a CAR and/or an exogenous TCR, and the CAR and/or exogenous TCR target CD19 and CD3. The engineered immune cell can express a CAR and/or an exogenous TCR, and the CAR and/or exogenous TCR target CD19 and CD5. The engineered immune cell can comprise a cytokine-related signaling pathway that is enhanced. The cytokine-related signaling pathway can comprise a related signaling pathway of a cytokine selected from a group consisting of IL-2, IL-3, IL-4, IL-6, IL-7, IL-8, IL-10, IL-11, IL-12, IL-15, IL-17, IL-18, IL-21, or a combination thereof. Enhancing the cytokine-related signaling pathway can comprise introducing a gene encoding a cytokine and/or a receptor thereof, up-regulating a gene encoding a cytokine and/or a receptor thereof, or exogenously adding a cytokine, a receptor of cytokine that is introduced, or a combination thereof. The engineered immune cell can be a CAR-T cell having one or more characteristics selected from a group consisting of (a) gene expression an endogenous TCR being silenced; (b) expressing an element for cellular suicide; (c) normal expression of endogenous HLA-I and HLA-II genes; (d) normal expression or overexpression of endogenous HLA-A, HLA-B, HLA-C, HLA-E, HLA-F and/or HLA-G.

The engineered immune cell can comprise a CAR or an exogenous TCR targeting a CD19. The engineered immune cell can comprise a substance targeting a T cell or NK cell. For example, the engineered immune cell can comprise a CAR targeting a T cell and/or NK cell. The engineered immune cell can comprise a bispecific CAR targeting both (i) a CD19 and (ii) a T cell and/or NK cell marker such as CD2, CD3, CD5 or CD7. In some cases, the engineered immune cell can comprise a bispecific CAR targeting both (i) a CD19 and (ii) CD7. In some cases, the engineered immune cell can comprise a bispecific CAR targeting both (i) a CD19 and (ii) CD2. In some cases, the engineered immune cell can comprise a bispecific CAR targeting both (i) a CD19 and (ii) CD3. In some cases, the engineered immune cell can comprise a bispecific CAR targeting both (i) a CD19 and (ii) CD5. In some cases, the substance is an antibody. The antibody target both T cell and NK cell can be TH-69, 3Ale, 3Alf, T3-3A1, RFT2, SDZ214-380 (SDZCHH380), CD7-6B7, 124-1D1, RPA-2.10, TS1/8, OKT11, AB75, 3E11, BH1, Lo-CD2a, UCHT2, L17F12, 4C7, CD5-5D7, UCHT1, OKT3, or BL1a.

The CAR-T cell provided herein can be a universal CAR-T cell. The CAR-T cell can express a chimeric antigen receptor CAR that targets a tumor cell marker such as CD19 and the binding of the T cell receptor to PD-1 is inhibited. The CAR-T cell can target a tumor cell marker and an immune cell marker such as CD2, CD3, CD5 or CD7. The endogenous TCR expression in the CAR-T cells provided herein can be knocked out by gene editing technology. Upon knocking out the endogenous TCRs of the CAR-T cells, the normal cells may not be recognized and killed by the CAR-T cells during the allogeneic infusion. The GVHD reaction may be inhibited. Moreover, targeting tumor cells through CD19 while eliminating host T cells and/or NK cells through CD2, CD3, CD5 or CD7 can avoid host versus graft (HVG) and/or NK killing and improve the survival and anti-tumor effect of the allogeneic CAR-T cells in the recipient. The CAR-T can further comprise a suicide gene switch (e.g., an inducible cell death moiety). The CAR-T cells can be inactivated or removed by turning on the suicide gene switch (e.g., binding of an activator to the inducible cell death moiety) to reduce the side effects of the CAR-T cell therapy. For example, a CAR provided herein can have a structure of CD19 scFv-CD7 scFv-Hinge-TM-CD28/41BB-CD3ζ, wherein the CD7 scFv fragment is a monoclonal 3Ale antibody, the heavy and light chain variable regions are joined by a GS linker, and the CD19 scFV fragment is the heavy and light chain variable region of an anti-CD19 antibody linked by a GS linker. The CAR can also include a hinge region and a transmembrane region in tandem, human CD28 and/or 41BB intracellular co-stimulatory elements, as well as human CD3 intracellular domain. In some cases of the present disclosure, a gene fragment of a CAR construct CD19 scFv-CD7 scFv-Hinge-TM-CD28/41BB-CD3 can be inserted into a lentiviral vector, and the recombinant vector can be packaged into viral particles in 293T cells. To prepare universal CAR-T cells, T cells can be isolated from peripheral blood mononuclear cells, and after activation, some endogenous genes (e.g., CD7, TCR and PD-1 genes) can be knocked out by gene editing technologies such as CRISPR/CAS technology. Next, T cells can be infected by the viral particles containing the CAR construct describe herein to express the CAR. The prepared CAR-T cells can be used to detect the infection efficiency and gene editing efficiency of CAR by flowcytometry.

The engineered immune cell may have one or more characteristics described herein: (a) the expression of the endogenous CD7, CD5, CD3 or CD2 gene of the engineered immune cell is silenced; (b) the PD-1 gene expression of the engineered immune cell is silenced; (c) the TCR gene expression of the engineered immune cell is silenced; (d) the engineered immune cell expresses a cytokine or cytokine receptor complex and the pSTAT5 signaling level is upregulated; (e) the engineered immune cell expresses an exogenous inducible cell death moiety; (f) the first CAR, and/or the second CAR in the engineered immune cell is co-expressed with the inducible cell death moiety.

The engineered immune cell may comprise two different CARs, each having a different antigen binding domain target a different antigen. The engineered immune cell may comprise a single CAR, which further comprises two antigen binding domains targeting two different antigens. In some cases, a first CAR, and/or a second CAR is linked to an inducible cell death moiety and/or an enhancer moiety by a self-cleaving element. In some cases, the enhancer moiety is a cytokine or cytokine complex. Examples of cytokines or cytokine complexes include IL2, IL7, IL15, membrane-bound IL15 (mbIL15 or mb15), and a constitutive activating cytokine receptor such as an IL7 receptor (C7R). As used herein, “mbIL” and “mb” are used interchangeably to refer to a membrane-bound interleukin factor, for example, mbIL7 or mb7, and mbIL17 or mb17.

The engineered immune cell described herein may have the following characteristics: (a) the engineered immune cell expresses a CAR and/or an exogenous TCR, and the CAR and/or exogenous TCR targets tumor cell markers such as CD19; and (b) the cytokine-associated signaling pathway is enhanced. The engineered immune cell may be (i) chimeric antigen receptor T cells (CAR-T cells); (ii) chimeric antigen receptor NK cells (CAR-NK cells); or (iii) Exogenous T cell receptor (TCR) T cells (TCR-T cells). The engineered immune cell can be a CAR-T cell, preferably a universal CAR-T cell (UCAR-T cell). The “cytokine-related signaling pathway,” as used herein, refers to a signaling pathway initiated by the cytokine binding to the corresponding receptor, converting the extracellular signal into an intracellular signal, which is then amplified, dispersed, and regulated by a signal cascade. A series of cellular responses can be produced. In some cases, the cytokine-related signaling pathway comprises a related signaling pathway of a cytokine selected from the group consisting of IL-2, IL-3, IL-4, IL-6, IL-7, IL-8, IL-10, IL-11, IL-12, IL-15, IL-17, IL-18, IL-21, 25 or a combination thereof.

The engineered immune cell can comprise a bispecific CAR (or a dual CAR). For example, the bispecific CAR can comprise both a first antigen binding domain and a second antigen binding domain. The first antigen binding domain and the second antigen binding domain can be linked via a linker. The linker may not comprise a self-cleaving peptide. The first antigen binding domain or the second antigen binding can be a scFv.

The first antigen binding domain and the second antigen binding domain can be arranged, from amino terminus to carboxyl terminus, as: (i) VL2-VH1-VL1-VH2; (ii) VH2-VL1-VH1-VL2; (iii) VL1-VH2-VL2-VH1; or (iv) VH1-VL2-VH2-VL1, wherein VH1 is heavy chain variable domain of the first antigen binding domain, VL1 is light chain variable light domain of the first antigen binding domain, VH2 is heavy chain variable domain of the second antigen binding domain, and VL2 is light chain variable domain of the second antigen binding domain.

The first antigen binding domain and the second antigen binding domain can be arranged, from amino terminus to carboxyl terminus, as: (i) VL2-VH2-VL1-VH1; (ii) VL2-VH2-VH1-VL1; (iii) VL1-VH1-VL2-VH2; or (iv) VL1-VH1-VH2-VL1, wherein VH1 is heavy chain variable domain of the first antigen binding domain, VL1 is light chain variable light domain of the first antigen binding domain, VH2 is heavy chain variable domain of the second antigen binding domain, and VL2 is light chain variable domain of the second antigen binding domain. The first antigen binding domain and the second antigen binding domain can bind to the immune cell antigen and the disease-associated antigen.

In some cases, the engineered immune cell may not comprise a bispecific CAR. For example, an individual CAR of the engineered immune cell can comprise only the first antigen binding domain and an additional individual CAR of the engineered immune cell can comprise only the second antigen binding domain.

The immune cell antigen can be a surface protein or a secreted protein of an immune cell. The immune cell can be an NK cell, a T cell, a monocyte, a macrophage or a granulocyte. The immune cell antigen can be selected from the group consisting of CD2, CD3, CD5, and CD7.

The disease-associated antigen can be a tumor-associated antigen. The tumor-associated antigen can be CD19. In some cases, the first antigen binding domain can bind to an immune cell antigen selected from the group consisting of CD2, CD3, CD5, and CD7, and the second antigen binding domain can bind to CD19. In some cases, the first antigen binding domain can bind to CD7, and the second antigen binding domain can bind to CD19. In some cases, the first antigen binding domain can bind to CD2, and the second antigen binding domain can bind to CD19. In some cases, the first antigen binding domain can bind to CD3, and the second antigen binding domain can bind to CD19. In some cases, the first antigen binding domain can bind to CD5, and the second antigen binding domain can bind to CD19. The expression of one or more endogenous human leukocyte antigen (HLA) genes of the engineered immune cell can remain intact. The expression of endogenous HLA-I and/or HLA-II genes of the engineered immune cell can remain intact. The expression of endogenous HLA-A, HLA-B, HLA-C, HLA-E, HLA-F and/or HLA-G genes of the engineered immune cell can remain intact. The expression of one or more endogenous HLA genes of the engineered immune cell can be upregulated. The expression of endogenous HLA-A, HLA-B, HLA-C, HLA-E, HLA-F and/or HLA-G genes of the engineered immune cell can be upregulated.

In various embodiments, the engineered immune cell is a T cell, an NKT cell or an NK cell. In some cases, the engineered immune cell is derived from a stem cell. The stem cell can be a hematopoietic stem cell (HSC) or an induced pluripotent stem cell (iPSC).

A cell (e.g., an engineered immune cell) provided herein can comprise one or more chimeric antigen receptors (CARs) comprising a binding moiety, where the binding moiety can comprise an antigen binding domain capable of binding to an immune cell antigen. Each CAR of the one or more CARs can further comprise a transmembrane domain and an intracellular signaling domain. The cell can further comprise an enhancer moiety capable of enhancing one or more activities of the cell, where an endogenous T cell receptor (TCR) of the cell may be inactivated.

The enhancer moiety can enhance one or more activities of the cell. The enhancer moiety can be configured to constitutively enhance the one or more activities of the cell. The enhancer moiety can be configured to constitutively upregulate one or more intracellular signaling pathways of the cell. For example, the one or more intracellular signaling pathways can be one or more cytokine signaling pathways. The enhancer moiety can be a cytokine or a cytokine receptor. The enhancer moiety can be selected from the group consisting of IL-2, IL-3, IL-4, IL-6, IL-7, IL-8, IL-10, IL-11, IL-12, IL-15, IL-17, IL-18, IL-21, IL-23, PD-1, PD-L1, CD122, CSF1R, CTAL-4, TIM-3, CCL21, CCL19, TGFR beta, receptors for the same, functional fragments thereof, functional variants thereof, and combinations thereof.

The cell can further comprise an inducible cell death moiety capable of effecting death of the cell upon contacting the inducible cell death moiety with a cell death activator. The inducible cell death moiety can be selected from the group consisting of rapaCasp9, iCasp9, HSV-TK, CD20, ΔCD20, mTMPK, ΔCD19, RQR8, Her2t, CD30, BCMA, and EGFRt. For example, the inducible cell death moiety can be EGFRt, and the cell death activator can be an antibody or an antigen binding fragment thereof that binds EGFRt. For another example, the inducible cell death moiety can be HSV-TK, and the cell death activator can be GCV. For another example, the inducible cell death moiety can be iCasp9, and the cell death activator can be AP1903.

A gene encoding an endogenous surface marker of the cell can be inactivated, where the endogenous surface marker may be capable of binding to the first antigen binding domain when expressed. The endogenous surface marker can be CD2, CD3, CD5, or CD7. In some cases, the endogenous surface marker can be CD7. In some cases, the endogenous surface marker can be CD2. In some cases, the endogenous surface marker can be CD3. In some cases, the endogenous surface marker can be CD5.

### Antigen Binding Domain

The engineered immune cell can comprise a first antigen binding domain and a second antigen binding domain. In some cases, a single or individual CAR of the engineered immune cell comprises both the first antigen binding domain and the second antigen binding domain. In some cases, two CARs of the engineered immune cell comprise the first antigen binding domain and the second antigen binding domain with each CAR contains only one antigen binding domain. In some cases, the first antigen binding domain can target an immune cell antigen and the second antigen binding domain can target a disease-associated antigen. In some cases, the first antigen binding domain can target CD7 and the second antigen binding domain can target CD19. In some cases, the first antigen binding domain can target CD2 and the second antigen binding domain can target CD19. In some cases, the first antigen binding domain can target CD3 and the second antigen binding domain can target CD19. In some cases, the first antigen binding domain can target CD5 and the second antigen binding domain can target CD19. The antigen binding domain can be a Fab, F(ab′)2, single domain antibody, single chain Fv (scFv), centyrin, darpin, or other polypeptides with antigen binding specificities.

The antigen binding domain can target an immune cell antigen. Examples of immune cell antigen include, but are not limited to, CD2, CD3, CD5, and CD7. In some cases, the immune cell antigen is a cell marker expressed on both T cells and NK cells, including, but not limited to, CD2, and CD7.

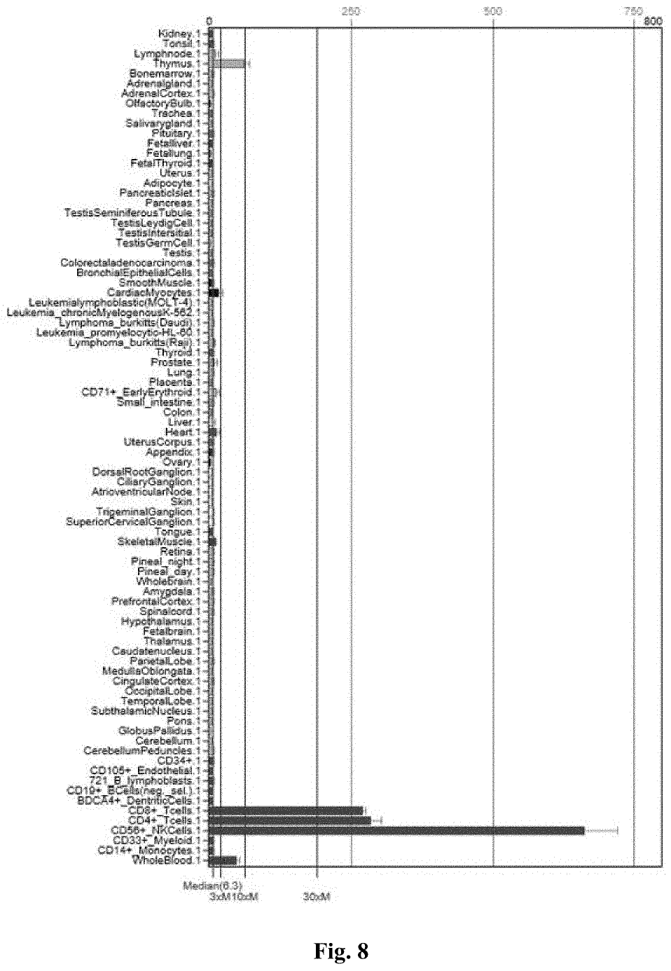

In some cases, the antigen binding domain can target CD7. CD7 is a transmembrane protein and is a member of the immunoglobulin superfamily. CD7 proteins are expressed on the surface of mature T cells and NK cells as well as their precursor cells. CD7 can bind to its ligand K12/SECTM1 and function as a co-stimulatory effecter on T cell activation. In mice, CD7 knockout T cell precursors can develop into normal T cells with only a slight effect on T cell effector function. More than 90% of T-cell acute lymphoblastic leukemia (T-ALL) can express CD7, and therefore CD7 can be a marker for T-ALL. Moreover, CD7 can also be expressed in NK lymphoma, T-cell lymphoma/leukemia, chronic myelogenous leukemia, acute myeloid leukemia, and lymphocyte-rich thymoma. An example tissue distribution of CD7 expression is shown in FIG. 8.

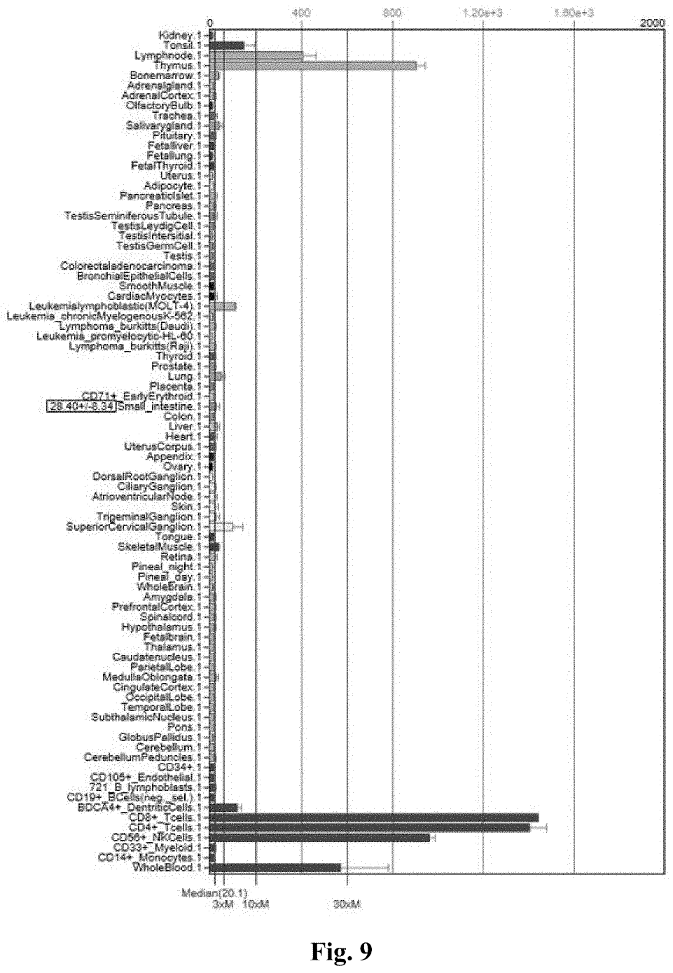

In some cases, the antigen binding domain can target CD2. Similar to CD7, CD2 adhesion molecules may express on all peripheral blood T cells and natural killer cells, but not on B lymphocytes. The CD2 extracellular domain contains an immunoglobulin-like domain that mediates homodimerization. Binding of CD2 to CD58 (LFA-3) or CD48 can help T cells adhere to antigen presenting cells, triggering signal transduction of T cell receptors for antigen binding. The function of CD2 may be similar to other T cell costimulatory receptors (such as CD28). CD2 knockout mice can have normal immune function. CD2 expression in cells of T-ALL, T-cell lymphoma/leukemia, acute promyelocytic leukemia (microparticle variant), systemic mastocytosis, mast cell disease, thymoma, and acute myeloid lymphoma and NK cell leukemia. An example tissue distribution of CD2 expression is shown in FIG. 9.

In some cases, the antigen binding domain can target CD5. CD5 includes a scavenger receptor cysteine-rich protein domain, and it expresses on the surface of T cells and in a subset of murine B cells known as B-1 cells. T cells express higher levels of CD5 than B cells and CD5 has been used as a T-cell marker. CD5 is upregulated on T cells upon activation. It may also mitigate activating signals from the BCR so that the B-1 cells can be activated by stimuli. There is no confirmed ligand for CD5, but evidence has shown that CD72, a C-type lectin, may be a ligand of CD5. About 76% of T-cell neoplasms are reported to express CD5, and it is also found in chronic lymphocytic leukemia and mantle cell lymphoma (both being B cell malignancies).

The antigen binding domain can target a disease-associated antigen. In some cases, the antigen binding domain can target CD19. CD19 is a 95 kDa glycoprotein on the surface of B cells that begins to express from the early development of B cells until it differentiates into plasma cells. CD19 is a member of the immunoglobulin (Ig) superfamily and is involved in the regulation of the signal transduction process of B cell receptors as one of the constituent elements of the B cell surface signal transduction complex. In a CD19-deficient mouse model, the number of B cells in peripheral lymphoid tissue can be significantly reduced, and the response to vaccines and mitogens is also reduced, accompanied by a decrease in serum Ig levels. It can be generally believed that the expression of CD19 is restricted to the B-cell lineage and not to the surface of pluripotent hematopoietic stem cells. CD19 can also be expressed on the surface of most B cell lymphomas, mantle cell lymphomas, ALLs, CLLs, hairy cell leukemias, and some acute myeloid leukemia cells. CD19 can be a target for immunotherapy in the treatment of leukemia/lymphoma. CD19 may not be expressed on most normal cell surfaces other than B cells, including pluripotent hematopoietic stem cells. This feature can make CD19 a safe therapeutic target for autoimmune diseases because the risk of irreversible bone marrow toxicity damage can be minimized.

The antigen binding domain provided herein can have a structure shown as VH-VL or V_{L}-V_{H}, wherein V_{H} is a heavy chain variable region of an antibody; V_{L} is a light chain variable region of an antibody; “-” is a linker peptide (or flexible linker) or a peptide bond.

In some embodiments, the antigen binding domain targets CD19. In some embodiments, the antigen binding domain that targets CD19 comprises the heavy chain variable region and the light chain variable region.

In some embodiments, the heavy chain variable region of the antigen binding domain that targets CD19 comprises an amino acid sequence having at least 90% identity with any one selected the group consisting of SEQ ID NO. 1-10. In some embodiments, the heavy chain variable region of the antigen binding domain that targets CD19 comprises an amino acid sequence having at least 91% identity with any one selected the group consisting of SEQ ID NO. 1-10. In some embodiments, the heavy chain variable region of the antigen binding domain that targets CD19 comprises an amino acid sequence having at least 92% identity with any one selected the group consisting of SEQ ID NO. 1-10. In some embodiments, the heavy chain variable region of the antigen binding domain that targets CD19 comprises an amino acid sequence having at least 93% identity with any one selected the group consisting of SEQ ID NO. 1-10. In some embodiments, the heavy chain variable region of the antigen binding domain that targets CD19 comprises an amino acid sequence having at least 94% identity with any one selected the group consisting of SEQ ID NO. 1-10. In some embodiments, the heavy chain variable region of the antigen binding domain that targets CD19 comprises an amino acid sequence having at least 95% identity with any one selected the group consisting of SEQ ID NO. 1-10. In some embodiments, the heavy chain variable region of the antigen binding domain that targets CD19 comprises an amino acid sequence having at least 96% identity with any one selected the group consisting of SEQ ID NO. 1-10. In some embodiments, the heavy chain variable region of the antigen binding domain that targets CD19 comprises an amino acid sequence having at least 97% identity with any one selected the group consisting of SEQ ID NO. 1-10. In some embodiments, the heavy chain variable region of the antigen binding domain that targets CD19 comprises an amino acid sequence having at least 98% identity with any one selected the group consisting of SEQ ID NO. 1-10. In some embodiments, the heavy chain variable region of the antigen binding domain that targets CD19 comprises an amino acid sequence having at least 99% identity with any one selected the group consisting of SEQ ID NO. 1-10. In some embodiments, the heavy chain variable region of the antigen binding domain that targets CD19 comprises an amino acid sequence of any one selected the group consisting of SEQ ID NO. 1-10.

In some embodiments, the light chain variable region of the antigen binding domain that targets CD19 comprises an amino acid sequence having at least 90% identity with any one selected the group consisting of SEQ ID NO. 11-17. In some embodiments, the light chain variable region of the antigen binding domain that targets CD19 comprises an amino acid sequence having at least 91% identity with any one selected the group consisting of SEQ ID NO. 11-17. In some embodiments, the light chain variable region of the antigen binding domain that targets CD19 comprises an amino acid sequence having at least 92% identity with any one selected the group consisting of SEQ ID NO. 11-17. In some embodiments, the light chain variable region of the antigen binding domain that targets CD19 comprises an amino acid sequence having at least 93% identity with any one selected the group consisting of SEQ ID NO. 11-17. In some embodiments, the light chain variable region of the antigen binding domain that targets CD19 comprises an amino acid sequence having at least 94% identity with any one selected the group consisting of SEQ ID NO. 11-17. In some embodiments, the light chain variable region of the antigen binding domain that targets CD19 comprises an amino acid sequence having at least 95% identity with any one selected the group consisting of SEQ ID NO. 11-17. In some embodiments, the light chain variable region of the antigen binding domain that targets CD19 comprises an amino acid sequence having at least 96% identity with any one selected the group consisting of SEQ ID NO. 11-17. In some embodiments, the light chain variable region of the antigen binding domain that targets CD19 comprises an amino acid sequence having at least 97% identity with any one selected the group consisting of SEQ ID NO. 11-17. In some embodiments, the light chain variable region of the antigen binding domain that targets CD19 comprises an amino acid sequence having at least 98% identity with any one selected the group consisting of SEQ ID NO. 11-17. In some embodiments, the light chain variable region of the antigen binding domain that targets CD19 comprises an amino acid sequence having at least 99% identity with any one selected the group consisting of SEQ ID NO. 11-17. In some embodiments, the light chain variable region of the antigen binding domain that targets CD19 comprises an amino acid sequence of any one selected the group consisting of SEQ ID NO. 11-17.

In some embodiments, the heavy chain variable region of the antigen binding domain that targets CD19 is a humanized heavy chain variable region. In some embodiments, the humanized heavy chain variable region comprises an amino acid sequence having a structure of formula I:

FH1-X1-FH2-X2-FH3-X3-FH4 (I).

In some embodiments, the FH1 comprises an amino acid of any one selected from SEQ ID NO. 18, 34, 35 or 36. In some embodiments, the FH2 comprises an amino acid of any one selected from SEQ ID NO. 19 or 37. In some embodiments, the FH3 an amino acid of any one selected from SEQ ID NO. 20, 38, 39, 40, 41, 42, 43, or 44. In some embodiments, the FH4 comprises an amino acid of any one selected from SEQ ID NO. 21 or 45.

In some embodiments, the FH1 comprises an amino acid identical to or comprising 1-3 amino acid residue addition, deletion, or substitutions relative to SEQ ID NO. 34, the FH2 comprises an amino acid identical to or comprising 1-3 amino acid residue addition, deletion, or substitutions relative to SEQ ID NO. 37, the FH3 comprises an amino acid identical to or comprising 1-3 amino acid residue addition, deletion, or substitutions relative to SEQ ID NO. 38, the FH4 comprises an amino acid identical to or comprising 1-3 amino acid residue addition, deletion, or substitutions relative to SEQ ID NO. 45.

In some embodiments, the FH1 comprises an amino acid identical to or comprising 1-3 amino acid residue addition, deletion, or substitutions relative to SEQ ID NO. 34, the FH2 comprises an amino acid identical to or comprising 1-3 amino acid residue addition, deletion, or substitutions relative to SEQ ID NO. 37, the FH3 comprises an amino acid identical to or comprising 1-3 amino acid residue addition, deletion, or substitutions relative to SEQ ID NO. 39, the FH4 comprises an amino acid identical to or comprising 1-3 amino acid residue addition, deletion, or substitutions relative to SEQ ID NO. 45.

In some embodiments, the FH1 comprises an amino acid identical to or comprising 1-3 amino acid residue addition, deletion, or substitutions relative to SEQ ID NO. 34, the FH2 comprises an amino acid identical to or comprising 1-3 amino acid residue addition, deletion, or substitutions relative to SEQ ID NO. 37, the FH3 comprises an amino acid identical to or comprising 1-3 amino acid residue addition, deletion, or substitutions relative to SEQ ID NO. 40, the FH4 comprises an amino acid identical to or comprising 1-3 amino acid residue addition, deletion, or substitutions relative to SEQ ID NO. 45.

In some embodiments, the FH1 comprises an amino acid identical to or comprising 1-3 amino acid residue addition, deletion, or substitutions relative to SEQ ID NO. 34, the FH2 comprises an amino acid identical to or comprising 1-3 amino acid residue addition, deletion, or substitutions relative to SEQ ID NO. 19, the FH3 comprises an amino acid identical to or comprising 1-3 amino acid residue addition, deletion, or substitutions relative to SEQ ID NO. 40, the FH4 comprises an amino acid identical to or comprising 1-3 amino acid residue addition, deletion, or substitutions relative to SEQ ID NO. 45.

In some embodiments, the FH1 comprises an amino acid identical to or comprising 1-3 amino acid residue addition, deletion, or substitutions relative to SEQ ID NO. 35, the FH2 comprises an amino acid identical to or comprising 1-3 amino acid residue addition, deletion, or substitutions relative to SEQ ID NO. 19, the FH3 comprises an amino acid identical to or comprising 1-3 amino acid residue addition, deletion, or substitutions relative to SEQ ID NO. 41, the FH4 comprises an amino acid identical to or comprising 1-3 amino acid residue addition, deletion, or substitutions relative to SEQ ID NO. 21.

In some embodiments, the FH1 comprises an amino acid identical to or comprising 1-3 amino acid residue addition, deletion, or substitutions relative to SEQ ID NO. 18, the FH2 comprises an amino acid identical to or comprising 1-3 amino acid residue addition, deletion, or substitutions relative to SEQ ID NO. 19, the FH3 comprises an amino acid identical to or comprising 1-3 amino acid residue addition, deletion, or substitutions relative to SEQ ID NO. 20, the FH4 comprises an amino acid identical to or comprising 1-3 amino acid residue addition, deletion, or substitutions relative to SEQ ID NO. 21.

In some embodiments, the FH1 comprises an amino acid identical to or comprising 1-3 amino acid residue addition, deletion, or substitutions relative to SEQ ID NO. 18, the FH2 comprises an amino acid identical to or comprising 1-3 amino acid residue addition, deletion, or substitutions relative to SEQ ID NO. 37, the FH3 comprises an amino acid identical to or comprising 1-3 amino acid residue addition, deletion, or substitutions relative to SEQ ID NO. 42, the FH4 comprises an amino acid identical to or comprising 1-3 amino acid residue addition, deletion, or substitutions relative to SEQ ID NO. 21.

In some embodiments, the FH1 comprises an amino acid identical to or comprising 1-3 amino acid residue addition, deletion, or substitutions relative to SEQ ID NO. 18, the FH2 comprises an amino acid identical to or comprising 1-3 amino acid residue addition, deletion, or substitutions relative to SEQ ID NO. 36, the FH3 comprises an amino acid identical to or comprising 1-3 amino acid residue addition, deletion, or substitutions relative to SEQ ID NO. 43, the FH4 comprises an amino acid identical to or comprising 1-3 amino acid residue addition, deletion, or substitutions relative to SEQ ID NO. 21.

In some embodiments, the FH1 comprises an amino acid identical to or comprising 1-3 amino acid residue addition, deletion, or substitutions relative to SEQ ID NO. 18, the FH2 comprises an amino acid identical to or comprising 1-3 amino acid residue addition, deletion, or substitutions relative to SEQ ID NO. 19, the FH3 comprises an amino acid identical to or comprising 1-3 amino acid residue addition, deletion, or substitutions relative to SEQ ID NO. 44, the FH4 comprises an amino acid identical to or comprising 1-3 amino acid residue addition, deletion, or substitutions relative to SEQ ID NO. 21.

In some embodiments, X1, X2, and X3 represent heavy chain CDRs of the antigen binding domain that targets CD19. The heavy chain CDRs used as the X1, X2 and X3 in the structure of formula I can be derived from any suitable monoclonal anti-CD19 antibody known in the art. In some embodiments, the monoclonal antibody of CD19 is selected from the group consisting of HD37, FMC63, 4G7, J4.119, B43, B4, HD237 and BU12. In some embodiments, X1, X2 and X3 may be derived from monoclonal FMC63 antibody.

In some embodiments, the X1 comprises an amino acid identical to or comprising 1-3 amino acid residue addition, deletion, or substitutions relative to SEQ ID NO. 26. In some embodiments, the X2 comprises an amino acid identical to or comprising 1-3 amino acid residue addition, deletion, or substitutions relative to SEQ ID NO. 27 or 28. In some embodiments, the X3 comprises an amino acid identical to or comprising 1-3 amino acid residue addition, deletion, or substitutions relative to SEQ ID NO. 29.

In some embodiments, the X1 comprises an amino acid identical to or comprising 1-3 amino acid residue addition, deletion, or substitutions relative to SEQ ID NO. 26, the X2 comprises an amino acid identical to or comprising 1-3 amino acid residue addition, deletion, or substitutions relative to SEQ ID NO. 27 and the X3 comprises an amino acid identical to or comprising 1-3 amino acid residue addition, deletion, or substitutions relative to SEQ ID NO. 29. In some embodiments, the X1 comprises an amino acid identical to or comprising 1-3 amino acid residue addition, deletion, or substitutions relative to SEQ ID NO. 26, the X2 comprises an amino acid identical to or comprising 1-3 amino acid residue addition, deletion, or substitutions relative to SEQ ID NO. 28 and the X3 comprises an amino acid identical to or comprising 1-3 amino acid residue addition, deletion, or substitutions relative to SEQ ID NO. 29.

In some embodiments, the light chain variable region of the antigen binding domain that targets CD19 is a humanized light chain variable region. In some embodiments, the humanized light chain variable region comprises an amino acid sequence having a structure of formula II:

FL1-Y1-FL2-Y2-FL3-Y3-FL4 (II)

In some embodiments, the FL1 comprises an amino acid of any one selected from SEQ ID NO. 22, or 46. In some embodiments, the FL2 comprises an amino acid of any one selected from SEQ ID NO. 23, 47 or 48. In some embodiments, the FL3 comprises an amino acid of any one selected from SEQ ID NO. 24, 49, 50, 51 or 52. In some embodiments, the FL4 comprises an amino acid of SEQ ID NO. 25.

In some embodiments, the FL1 comprises an amino acid identical to or comprising 1-3 amino acid residue addition, deletion, or substitutions relative to SEQ ID NO. 22, the FL2 comprises an amino acid identical to or comprising 1-3 amino acid residue addition, deletion, or substitutions relative to SEQ ID NO. 47, the FL3 comprises an amino acid identical to or comprising 1-3 amino acid residue addition, deletion, or substitutions relative to SEQ ID NO. 49 and the FL4 comprises an amino acid identical to or comprising 1-3 amino acid residue addition, deletion, or substitutions relative to SEQ ID NO. 25.

In some embodiments, the FL1 comprises an amino acid identical to or comprising 1-3 amino acid residue addition, deletion, or substitutions relative to SEQ ID NO. 22, the FL2 comprises an amino acid identical to or comprising 1-3 amino acid residue addition, deletion, or substitutions relative to SEQ ID NO. 47, the FL3 comprises an amino acid identical to or comprising 1-3 amino acid residue addition, deletion, or substitutions relative to SEQ ID NO. 50 and the FL4 comprises an amino acid identical to or comprising 1-3 amino acid residue addition, deletion, or substitutions relative to SEQ ID NO. 25.

In some embodiments, the FL1 comprises an amino acid identical to or comprising 1-3 amino acid residue addition, deletion, or substitutions relative to SEQ ID NO. 22, the FL2 comprises an amino acid identical to or comprising 1-3 amino acid residue addition, deletion, or substitutions relative to SEQ ID NO. 47, the FL3 comprises an amino acid identical to or comprising 1-3 amino acid residue addition, deletion, or substitutions relative to SEQ ID NO. 51 and the FL4 comprises an amino acid identical to or comprising 1-3 amino acid residue addition, deletion, or substitutions relative to SEQ ID NO. 25.

In some embodiments, the FL1 comprises an amino acid identical to or comprising 1-3 amino acid residue addition, deletion, or substitutions relative to SEQ ID NO. 46, the FL2 comprises an amino acid identical to or comprising 1-3 amino acid residue addition, deletion, or substitutions relative to SEQ ID NO. 47, the FL3 comprises an amino acid identical to or comprising 1-3 amino acid residue addition, deletion, or substitutions relative to SEQ ID NO. 24 and the FL4 comprises an amino acid identical to or comprising 1-3 amino acid residue addition, deletion, or substitutions relative to SEQ ID NO. 25.

In some embodiments, the FL1 comprises an amino acid identical to or comprising 1-3 amino acid residue addition, deletion, or substitutions relative to SEQ ID NO. 22, the FL2 comprises an amino acid identical to or comprising 1-3 amino acid residue addition, deletion, or substitutions relative to SEQ ID NO. 23, the FL3 comprises an amino acid identical to or comprising 1-3 amino acid residue addition, deletion, or substitutions relative to SEQ ID NO. 24 and the FL4 comprises an amino acid identical to or comprising 1-3 amino acid residue addition, deletion, or substitutions relative to SEQ ID NO. 25.

In some embodiments, the FL1 comprises an amino acid identical to or comprising 1-3 amino acid residue addition, deletion, or substitutions relative to SEQ ID NO. 46, the FL2 comprises an amino acid identical to or comprising 1-3 amino acid residue addition, deletion, or substitutions relative to SEQ ID NO. 48, the FL3 comprises an amino acid identical to or comprising 1-3 amino acid residue addition, deletion, or substitutions relative to SEQ ID NO. 52 and the FL4 comprises an amino acid identical to or comprising 1-3 amino acid residue addition, deletion, or substitutions relative to SEQ ID NO. 25.

In some embodiments, Y1, Y2, and Y3 represent light chain CDRs of the antigen binding domain that targets CD19. The light chain CDRs used as the Y1, Y2, and Y3 of the structure of formula I can be derived from any suitable monoclonal anti-CD19 antibody known in the art. In some embodiments, the monoclonal antibody of CD19 is selected from the group consisting of HD37, FMC63, 4G7, J4.119, B43, B4, HD237 and BU12. In some embodiments, Y1, Y2, and Y3 may be derived from monoclonal FMC63 antibody.

In some embodiments, the Y1 comprises an amino acid identical to or comprising 1-3 amino acid residue addition, deletion, or substitutions relative to SEQ ID NO. 30. In some embodiments, the Y2 comprises an amino acid identical to or comprising 1-3 amino acid residue addition, deletion, or substitutions relative to SEQ ID NO. 31. In some embodiments, the Y3 comprises an amino acid identical to or comprising 1-3 amino acid residue addition, deletion, or substitutions relative to SEQ ID NO. 32 or 33.

In some embodiments, the Y1 comprises an amino acid identical to or comprising 1-3 amino acid residue addition, deletion, or substitutions relative to SEQ ID NO. 30, the Y2 comprises an amino acid identical to or comprising 1-3 amino acid residue addition, deletion, or substitutions relative to SEQ ID NO. 31 and the Y3 comprises an amino acid identical to or comprising 1-3 amino acid residue addition, deletion, or substitutions relative to SEQ ID NO. 32. In some embodiments, the Y1 comprises an amino acid identical to or comprising 1-3 amino acid residue addition, deletion, or substitutions relative to SEQ ID NO. 30, the Y2 comprises an amino acid identical to or comprising 1-3 amino acid residue addition, deletion, or substitutions relative to SEQ ID NO. 31 and the Y3 comprises an amino acid identical to or comprising 1-3 amino acid residue addition, deletion, or substitutions relative to SEQ ID NO. 33.

In some embodiments, the sequence of the linker peptide or flexible linker comprises 2-6, preferably 3-4 consecutive (GGGGS) amino acid sequences.

In some embodiments, the antigen binding domain targets an immune cell antigen. In some embodiments, the immune cell antigen is CD7. In some embodiments, the immune cell antigen is CD2. In some embodiments, the immune cell antigen is CD3. In some embodiments, the immune cell antigen is CD5. In some embodiments, the monoclonal antibody of CD7 is selected from the group consisting of TH-69, 3Ale, 3Alf, T3-3A1, RFT2, CD7-6B7, 124-1D1, 4H9, SDZ214-380, or a combination thereof. In some embodiments, the amino acid sequence of V_{L} of the antigen binding domain targeting CD7 is as shown in SEQ ID NO.: 68, and the amino acid sequence of V_{H} of the antigen binding domain targeting CD7 is shown in SEQ ID NO.: 67. In some embodiments, the amino acid sequence of the antigen binding domain targeting CD7 is shown in any one of SEQ ID NO.: 69-78.

In some other embodiments, the monoclonal antibody to CD2 is selected from the group consisting of RPA-2.10, TS1/8, OKT11, AB75, 3E11, BH1, Lo-CD2a, or a combination thereof. In some other embodiments, the monoclonal antibody to CD3 is selected from the group consisting of UCHT1, OKT3, HIT3a, SK7 or a combination thereof. In some other embodiments, the monoclonal antibody to CD5 is selected from the group consisting of UCHT2, L17F12, 4C7, CD5-5D7, UCHT2, BL1a, or a combination thereof.

### Enhancer Moiety

The engineered immune cell provided herein can comprise an enhancer moiety. The enhancer moiety can regulate one or more activities of the engineered immune cell, for example, enhance or upregulate one or more signaling pathways to enhance or upregulate effector functions of the engineered immune cell. The signaling pathways can be a cytokine-related signaling pathway. The enhancer moiety can be a cytokine. The enhancer moiety can be a cytokine receptor.

The cytokine-related signaling pathway can comprise a related signaling pathway of a cytokine. Examples of cytokines include, but are not limited to, IL-2, IL-3, IL-4, IL-6, IL-7, IL-8, IL-10, IL-11, IL-12, IL-15, IL-17, IL-18, IL-21 and IL25. In some cases, the cytokine-related signaling pathway comprises a related signaling pathway of two or more cytokines, wherein the cytokines include: IL-2 and IL-7, IL-2 and IL-15. IL-7 and IL-15, 1L15 and IL21. The cellular response can include regulation of downstream gene expression, changes in intracellular enzyme activity, changes in cellular bone architecture, changes in DNA synthesis, promotion of gene transcription, regulation of immune cell differentiation, proliferation, and resistance to cell death. In some cases, the cytokine-related signaling pathway is enhanced comprising: introducing or up-regulating a gene encoding a cytokine and/or a receptor thereof, exogenously adding a cytokine, being introduced into a cytokine receptor, or a combination thereof. In some cases, up-regulating the gene encoding the cytokine and/or its receptor comprises up-regulating the level of transcription and/or translation of the encoding gene. In some cases, the enhanced cytokine-related signaling pathway can be achieved by one or more of the following methods: expressing a gene encoding the cytokine and/or its receptor in the immune cell, increasing the copy number of the gene encoding the cytokine and/or its receptor in the immune cell, engineering a regulatory sequence (e.g., a promoter) of the encoding gene to enhance transcription speed (e.g., transcriptional initiation rate), modifying a translational regulatory region of a messenger RNA carrying the encoded gene to enhance translational strength, modifying the coding gene itself to enhance mRNA stability, protein stability and to release protein feedback inhibition.

The cytokine-related signaling pathway can be enhanced by membrane expression of a cytokine and its receptor, secretion of a cytokine, enhancement of transcriptional regulation of a cytokine and/or its receptor, or a combination thereof. The membrane-expressed cytokine and its receptors can include: IL-15 and its receptor (e.g., mbIL15 fusion protein), IL-7 and its receptor (e.g., mbIL7 fusion protein), IL-17 and its receptor (e.g., mbIL17 fusion protein), IL-2 and its receptor (e.g., mbIL2 fusion protein), IL-21 and its receptor (e.g., mbIL21 fusion protein), constitute the activated IL-7 receptor (C7R), or a combination thereof. In some cases, the enhancer moiety comprised in the engineered immune cell is a secretive cytokine. The secretive cytokine can function with various mechanisms, for example, the secretive cytokine can be a trans-activating factor or a cis-activating factor. The secretive cytokines can include IL-2, IL-3, IL-4, IL-6, IL-7, IL-8, IL-10, IL-11, IL-12, IL-15, IL-17, IL-18, IL-21, or a combination thereof. In some cases, the enhancer is a membrane bound protein such as mbIL15, mbIL7, mbIL21 and mbIL2. In some cases, the enhancer moiety is constitutively active cytokine receptor downstream signaling protein such as STAT5 and STAT3. In some cases, the enhancer moiety is a constitutively active cytokine receptor such as constitutively active IL-7 receptor (C7R) or derivatives thereof. For example, the constitutively active cytokine receptor can be an engineered protein (e.g., referred to as “E3” in the present disclosure) where the ecto domain of C7R is replaced by a safety switch, such as EGFRt or truncated form of human epidermal growth factor receptor 2 (Her2t; see U.S. Patent Application Publication No. 20170267742A1) or other peptides described in the present disclosure. For another example, the constitutively active cytokine receptor can be an engineered protein (e.g., referred to as “E4” in the present disclosure) where the ectodomain of C7R is replaced by an immune cell inhibitor, such as CD47, CD24 or other peptides that inhibit killer or phagocytic immune cell function and protect therapeutic cells (e.g., the engineered immune cells described herein). In some cases, the cytokine can be a chemokine such as CCL21 and CCL19. Other non-limiting examples of chemokines that may be used include CCL27, CCL28, CCL20, CXCL9, CXCL10, CXCL11, CXCL16, CXCL13, CXCL5, CXCL6, CXCL8, CXCL12, CCL2, CCL8, CCL13, CCL25, CCL3, CCL4, CCL5, CCL7, CCL14, CCL15, CCL16, CCL23, CX3CL1, XCL1, XCL2, CCL1, CCL17, CCL22, CCL11, CCL24, CCL26, CXCL1, CXCL2, CXCL3 and CXCL7. In some cases, the enhancer moiety is a ligand of CCR7, which can function to enhance infiltration of T cells, NK cells or dendritic cells. CCR7 ligand includes, but not limited to, CCL21 and CCL19. In some cases, the enhancer moiety comprises co-expression of chemokines CCL21 and CCL19 for therapeutic use to treat lymphomas or other solid tumors.

In some situations, the engineered immune cell is used as a therapeutic agent to treat liquid tumors, and in such situations, the enhancer moiety can comprise any cytokine in any form as described herein. In some situations, the engineered immune cell is used as a therapeutic agent to treat solid tumors, and in such situations, the enhancer moiety can comprise any cytokine in any form and further comprise one or more chemokines.

In some cases, two cytokines may be used to enhance the cytokine-related signaling pathway in the engineered immune cell, including IL-2 and IL-7, IL-2 and IL-15, IL-7 and IL-15, and IL15 and IL21. The cytokine-related signaling pathway enhancement can comprise the expression of a polypeptide selected from the group consisting of a mbIL fusion protein, a constitutively active IL-7 receptor (C7R), an interleukin, or a combination thereof.

The enhancer moiety described herein can be interleukin 15 (IL-15) or IL-15 receptor. IL-15 is a 14-15kDs glycoprotein composed of 114 amino acids and belongs to the family of four helix bundle cytokines. IL-15 is structurally homologous to interleukin 2 (IL-2). IL-15 receptor comprises a high affinity IL-15 receptor alpha chain, an IL2/15 receptor beta chain, and a common gamma chain. Therefore, IL-15 may have some functions similar to IL-2, such as stimulating T cell activation and proliferation, enhancing NK cell killing activity and promoting B cell production of immunoglobulin. Recent studies have found that IL-15 may play a role in the differentiation, proliferation and activation of NK cells, NKT cells and intestinal epithelial cells. IL-15 and IL-17 may play a role in the regulation of CD8+ memory T cells. Studies have also shown that IL-15 can regulate the proliferation of CD8+ memory T cells and the survival cycle of NK cells through a mechanism, in which a cell expressing IL-15a chain receptor can present IL-15 to a cell expressing an IL-150 chain and a common gamma chain. IL-15 may also play a role in the non-immune system, such as regulation of skeletal muscle anabolism. The enhancer moiety described herein can be interleukin 7 (IL-7). IL-7 can promote the growth of pre-B cells, pro-B cells, B cells, and T cells. It can also promote growth and anti-apoptosis of B cells and T cells. IL-7 can play a role in the early differentiation and proliferation of thymus and the development and differentiation of dendritic cells. However, IL-7 may not have an enhanced effect on the killing activity of antigen-specific cytotoxic T lymphocytes. It can first transfer from the thymus to the peripheral blood, then induce thymocytes or peripheral blood lymphocytes to produce lymphokines, activate and enhance lymphokine-activated killer cell activity of LAK cells. CD8+ subpopulation can be the main effector cell of IL-7, and IL-7 Can also support memory CD8+ T cell expansion and survival. IL-7 can promote bone marrow tissue production. IL-7 not only can stimulate myeloid precursor cells and megakaryocytes to produce colony forming units and platelets, but also can restore the body from the immunosuppression of cyclophosphamide. At higher concentrations, it can also induce cytotoxicity that enhances macrophages, function as a synergistic factor for the production of CTL cells, NK cells, and activated monocytes, induce monocyte-macrophages to secrete various cytokines and promote the expression of inflammatory factors such as macrophage inflammatory protein alpha (MIP-alpha), MIP-β, IL-8 and monocyte chemoattractant protein-1 (MCP-1) and the like. By activating a large number of inflammatory factors produced by inflammatory cells, IL-7 not only can regulate the interaction between the components of the inflammatory process, but also enhance the inflammatory cytokine receptors (CCR) such as CCR1, CCR2 and CCR5. In addition, IL-7 can play a role in inducing immune responses. IL-7 can induce type I immune responses and increase the production of IFN-7 and IL2. IL-7 can synergize with IL12 to induce IFN-7 and T cell proliferation. IL-7 and transforming growth factor beta (TGFβ) can play a regulatory role and can be part of the immune regulatory mechanism. IL-7 not only can promote immune reconstitution of T cells, but also can induce up-regulation of T cell cycle and BCL-2 expression, which broadens the diversity and persistence of circulating T cell receptor pools and increases the number of CD4+ and CD8+ T cells. Moreover, for HIV antigens, expanded T cells can also secrete IL2 and IFN-γ, and have good antiviral function. Therefore, IL-7 can reverse the defects of HIV-specific T lymphocytes in proliferation, cytokine secretion and cell function.

The enhance moiety can regulate (e.g., activate) signal transducer and activator of transcription 5 (STAT5)-mediated signaling pathway. STAT5 can be widely present in the cytoplasm. When cytokines (e.g., IL2, IL7, IL15 and IL21) bind to the cytokine receptors, the receptor-coupled JAK is activated, thereby phosphorylating the Tyr residue at the C-terminus of the STAT5 protein. The phosphorylated STAT5 can form homologous or heterologous dimers through its SH2 region. The homologous or heterodimer can be transferred to the nucleus and bind to the target gene, thereby regulating the expression of the target gene including the cell regulatory factor and the anti-apoptotic gene. Activation of STAT5 can play a role in maintaining normal cell function and regulating cell proliferation and differentiation. Therefore, regulating the activity of STAT5 signaling pathway may regulate the survival and persistence of CAR-T cells described herein.

The enhancer moiety can be introduced into a cell (e.g., an immune cell or an engineered immune cell) by delivering a nucleic acid molecule encoding the enhancer moiety into the cell. The nucleic acid molecule can be a vector. The enhancer moiety can be a part of a fusion construct. A fusion protein or corresponding nucleic acid construct can have a structure as presented by a formula selected from: S-2A-L1-scFv-H-TM-C-CD3ζ-2A-L2-IL15-IL15Ra (A); S-2A-L1-scFv-H-TM-C-CD3ζ-2A-L2-IL15-IL15Ra-2A-L3-IL7 (B); S-2A-L1-scFv-H-TM-C-CD3ζ-2A-L2-C7R (C); S-2A-L1-scFv-H-TM-C-CD3ζ-2A-L2-IL7-IL7Ra (D); wherein: each “-” is independently a linker peptide or a peptide bond; S is a safety switch; 2A is an optional self-cleaving peptide; each of L1, L2 and L3 is independently null or a signal peptide sequence; C7R is as described above; scFv is an antigen binding domain; H is null or a hinge region; TM is a transmembrane domain; C is a costimulatory signaling molecule; CD3ζ is a cytoplasmic signaling sequence derived from CD3ζ; IL15 is interleukin 15, IL15Ra is IL-15 receptor a; IL7 is interleukin 7, IL7Ra is IL-7 receptor a; C7R is a constitutively activated IL-7 receptor.

The enhancer moiety can be part of a chimeric polypeptide. For example, the enhancer moiety can be linked to an inducible cell death moiety. The enhancer moiety can be linked to the inducible cell death moiety by a linker. The linker may not be cleaved. The linker may not comprise a self-cleaving peptide. In some other cases, the enhancer moiety and the inducible cell death moiety can be expressed in a cell from a same nucleic acid molecule and can be cleaved to form two polypeptides.

### Inducible Cell Death Moiety

The engineered immune cell described herein may comprise an inducible cell death moiety, also referred to as “suicide gene switch.” “suicide switch,” “safety switch,” or “cell suicide element.” The inducible cell death moiety can be used to effectively remove of the engineered immune cells (e.g., CAR-T cells) in vivo under the action of exogenous factors (e.g., drugs). The inducible cell death moiety described herein may be rapaCasp9, iCasp9, CD20 (and its mimotope), RQR8, Her2t, CD30, BCMA, EGFRt, HSV-TK, mTMPK and the like. iCasp9, CD20 (and its mimotope), RQR8, and HSV-TK may have the same ability to clear T cells, but rapaCasp9, iCasp9, RQR8, and CD20 (and their mimotope) may be faster in comparison with HSV-TK.

In some cases, an inducible cell death moiety is capable of effecting death of said cell upon contacting said inducible cell death moiety with a cell death activator. The inducible cell death moiety can be, for example, rapaCasp9, iCasp9, HSV-TK, CD20, ΔCD20, mTMPK, ΔCD19, RQR8, or EGFRt. In some cases, the inducible cell death moiety is EGFRt, and said cell death activator is an antibody or an antigen binding fragment thereof that binds EGFRt. In some cases, the inducible cell death moiety is HSV-TK, and said cell death activator is GCV. In some cases, the inducible cell death moiety is iCasp9, and said cell death activator is AP1903.

The inducible cell death moiety can be linked to an enhancer moiety and can be co-expressed in a cell as a chimeric polypeptide as described above.

### Graft Versus Host Disease (GVHD)

To prepare “off-the-shelf” allogeneic T cells for the treatment of malignant and infectious diseases, cell therapy by infusion of T cells can be designed to re-establish immunity against pathogens and malignancies. The amount of time required to produce the T cells with tumor-targeting properties with a sufficient number of T cells in vitro can be generally incompatible with the patient's therapeutic window. Furthermore, autologous T cells from patients with advanced disease may have impaired function and are tolerant to the desired antigen.

To address these issues, patients can be administered with allogeneic T cells but need to be prevented from immune-mediated rejection by host T cells by recognizing different major or minor histocompatibility antigens on the infused cells. Infusion of T cells without the expression of TCR alpha and beta chains and HLA-A molecules may not cause GVHD and HVG. Thus the T cells edited with CRISPR/CAS9 to delete TCR alpha chain and HLA-A molecular can serve as a source of universal effector donor cells. In some cases, knockdown of Beta-2-Microglobulin (B2M) may also prevent donor CAR-T cells from being attacked by the host T cells.

In some cases, the donor CAR-T cells may be still be attacked by host NK cells and affect the survival of CAR-T cells. Therefore, the present disclosure provides engineered immune cells which target tumor cells and host T cells and/or NK cells. The engineered immune cells described herein can scavenge host T cells and/or NK cells, and enhance the survival, persistence and expansion ability of CAR-T cells, thereby being more effective against tumor cells.

### Gene Editing

Various gene editing methods can be used in the present disclosure to make the engineered immune cells, including CRISPR, RNA interference technology, TALENs (transcription activator-like (TAL) effector nucleases) and Zinc finger nucleases (ZFNs).

In some cases, CRISPR/Cas9 system is used to edit the genes of the immune cells. For example, CRISPR/Cas9 system can be used to knockout endogenous TCRs or cell surface markers (e.g., CD2, CD3, CD5 or CD7) of the immune cells to generate the engineered immune cells for T cell therapy. The CRISPR/Cas9 (clustered regular interspaced short palindromic repeats)/Cas (CRISPR-associated) system is a natural immune system unique to prokaryotes that is resistant to viruses or exogenous plasmids. The Type II CRISPR/Cas system has been applied in many eukaryotic and prokaryotic organisms as a direct genome-directed genome editing tool. The development of the CRISPR/Cas9 system has revolutionized the ability of people to edit DNA sequences and regulate the expression levels of target genes, providing a powerful tool for accurate genome editing of organisms. The simplified CRISPR/Cas9 system can comprise Cas9 protein and gRNA. The principle of action is that gRNA forms a Cas9-gRNA complex with Cas9 protein through its own Cas9 handle, and the base complementary pairing sequence of gRNA in the Cas9-gRNA complex is paired with the target sequence of the target gene by the principle of base complementary pairing. Cas9 uses its own endonuclease activity to cleave the target DNA sequence. Compared to traditional genome editing techniques, the CRISPR/Cas9 system has several distinct advantages: ease of use, simplicity, low cost, programmability, and the ability to edit multiple genes simultaneously.

### Pharmaceutical Composition

The present disclosure also provides a pharmaceutical composition comprising an engineered immune cell described herein and a pharmaceutically acceptable carrier, diluent or excipient. In some embodiments, the pharmaceutical composition is a liquid composition. The pharmaceutical composition can be administered into a subject, for example, by injection. The concentration of the engineered immune cells in the preparation can be at least about 10², 10³, 10⁴, 10³, 10⁶, 10⁷, 10⁸, 10⁹, or more cells/ml. In some case, the concentration of the engineered immune cells in the preparation can be 1×10³-1×10⁸ cells/ml, or 1×10⁴-1×10⁷ cells/ml.

The pharmaceutical compositions of the present disclosure may comprise engineered immune cells as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure may be formulated for intravenous administration.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present disclosure to be administered can be determined by a physician with consideration of individual differences in age, weight, tumor size, extent of infection or metastasis, and condition of the patient (subject). It can generally be stated that a pharmaceutical composition comprising the engineered immune cells (e.g., CAR-T cells) described herein may be administered at a dosage of 10⁴ to 10⁹ cells/kg body weight, or in some cases, 10⁵ to 10⁶ cells/kg body weight, including all integer values within those ranges. T cell compositions may also be administered multiple times at these dosages. The cells can be administered by using infusion techniques. The optimal dosage and treatment regime for a particular patient can readily be determined by monitoring the patient for signs of disease and adjusting the treatment accordingly.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i.v.) injection, or intraperitoneally. In some embodiments, the T cell compositions of the present disclosure are administered to a patient by intradermal or subcutaneous injection. In some other embodiments, the T cell compositions of the present disclosure are preferably administered by i.v. injection. The compositions of T cells may be injected directly into a tumor, lymph node, or site of infection.

### Therapeutics

The present disclosure provides therapeutic applications with engineered immune cells (e.g., T cells or NK cells) transduced with a lentiviral vector (LV) encoding an expression cassette described herein. Transduced T cells or NK cells can target tumor cell markers (such as CD19) and activated T cell and/or NK cell consensus markers (such as CD2, CD3, CD5, CD7). The engineered immune cells can be used for allogeneic tumor treatment and can be prepared on a large scale.

Accordingly, the present disclosure also provides a method of stimulating a T cell mediated immune response to a target cell population or tissue of a subject (e.g., a mammal) comprising the step of administering to the subject an engineered immune cell (e.g., CAR-T cell) of the disclosure.

In some embodiments, the present disclosure provides a type of cell therapy comprising directly administering engineered universal CAR-T cells of the present disclosure to a patient in need thereof. The CAR-T cells of the present disclosure may have the endogenous TCR expression knocked out or silenced in the cells by gene editing technology. Inactivation of the endogenous TCRs can prevent killing of normal cells by the TCRs during the allogeneic infusion. The GVHD reaction may be prevented. The CAR-T cells targeting a tumor cell marker (such as CD19) and a marker for activated T cells and/or NK cells (such as CD2, CD3, CD5, CD7) can remove activated T cells and/or NK cells while scavenging tumor cells. In addition, host versus graft response (HVG) can also be prevented. The cell therapy provided herein can also improve the survival and anti-tumor effect of allogeneic CAR-T cells in the subject.

In some embodiments, provided herein is a method of treating or diagnosing a disease in a subject, comprising administering the pharmaceutical composition described herein to said subject.

The engineered immune cell in said pharmaceutical composition can be derived from an allogeneic immune cell. The engineered immune cell derived from said allogeneic immune cell may not induce graft versus host disease (GvHD) in said subject. The engineered immune cell in said pharmaceutical composition can be derived from an autologous immune cell.

The endogenous TCR of said engineered immune cell in said pharmaceutical composition may be functionally inactive. The engineered immune cell can reduce GVHD in said subject compared to an additional immune cell having a functionally active TCR. The disease can be a cancer. The cancer can be, for example, lymphoma or leukemia.

The CAR-T cells of the present disclosure can undergo robust in vivo cell expansion and can be extended. The CAR-mediated immune response can be part of a step of adoptive immunotherapy in which CAR-modified T cells can induce an immune response specific for the antigen-binding domain in the CAR. For example, anti-CD19 CAR-T cells elicit a specific immune response against cells expressing CD19.

The engineered immune cells provided herein can be used to treat cancers. Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the CARs of the present disclosure include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

In some embodiments, the antigen bind moiety portion of the CAR of the present disclosure is designed to treat a particular cancer. For example, the CAR designed to target CD19 can be used to treat cancers and disorders including but are not limited to pre-B ALL (pediatric indication), adult ALL, mantle cell lymphoma, diffuse large B-cell lymphoma, salvage post allogeneic bone marrow transplantation, and the like.

The present disclosure should not be construed to be limited to solely to the antigen targets and diseases disclosed herein. Rather, the present disclosure should be construed to include any antigenic target that is associated with a disease where a CAR can be used to treat the disease.

The cell therapy disclosed herein can be co-formulated with, and/or co-administered with, one or more additional therapeutic agents, e.g., one or more anti-cancer agents, cytotoxic or cytostatic agents, hormone treatment, vaccines, and/or other immunotherapies. In some embodiments, the engineered immune cells are administered in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies.

In certain embodiments, the methods and compositions described herein are administered in combination with one or more antibody molecules, chemotherapy, other anti-cancer therapy (e.g., targeted anti-cancer therapies, or oncolytic drugs), cytotoxic agents, immune-based therapies (e.g., cytokines), surgical and/or radiation procedures. Exemplary cytotoxic agents that can be administered in combination with include antimicrotubule agents, topoisomerase inhibitors, anti-metabolites, mitotic inhibitors, alkylating agents, anthracyclines, vinca alkaloids, intercalating agents, agents capable of interfering with a signal transduction pathway, agents that promote apoptosis, proteasome inhibitors, and radiation (e.g., local or whole body irradiation).

In certain embodiments, the combination therapy, is used in combination with a standard of cancer care chemotherapeutic agent including, but not limited to, anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cy-tosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydro-chloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), nab-paclitaxel (Abraxane®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Examples of alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

Examples of anthracyclines include, e.g., doxorubicin (Adriamycin® and Rubex®); bleomycin (Lenoxane®); daunorubicin (dauorubicin hydrochloride, daunomycin, and rubidomycin hydro-chloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetyl-ravidomycin. 100255 Examples of vinca alkaloids that can be used in combination with the cell therapy described herein, include, but are not limited to, vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®).

Examples of proteasome inhibitors that can be used in combination with the cell therapy described herein, include, but are not limited to, bortezomib (Velcade®); carfilzomib (PX-171-007, (S)-4-Methyl-N—((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)-pentanamide); marizomib (NPI-0052); ixazomib citrate (MLN-9708); delanzomib (CEP-18770); 0-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912); danoprevir (RG7227, CAS 850876-88-9); ixazomib (MLN2238, CAS 1072833-77-2); and (S)—N-[(phenylmethoxy)carbonyl]-L-leucyl-N-(1-formyl-3-methylbutyl)-L-Leucinamide (MG-132, CAS 133407-82-6).

In some embodiments, the cell therapy may be used in combination with a tyrosine kinase inhibitor (e.g., a receptor tyrosine kinase (RTK) inhibitor). Exemplary tyrosine kinase inhibitor include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-β inhibitor)), a RAF-1 inhibitor, a KIT inhibitor and a RET inhibitor. In some embodiments, the anti-cancer agent used in combination with the hedgehog inhibitor is selected from the group consisting of: axitinib (AG013736), bosutinib (SKI-606), cediranib (RE-CENTIN, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVAS-TIN®), rituximab (RITUXAN®), cetuximab (ERBITUX®), panitumumab (VECTIBIX®), ranibizumab (Lucentis®), nilotinib (TASIGNA®), sorafenib (NEXAVAR®), alemtuzumab (CAM-PATH®), gemtuzumab ozogamicin (MYLOTARG®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EKB-569), vandetanib (zactima), WZ3146, WZ4002, WZ8040, ABT-869 (linifanib), AEE788, AP24534 (ponatinib), AV-951 (tivozanib), axitinib, BAY 73-4506 (regorafenib), brivanib alaninate (BMS-582664), brivanib (BMS-540215), cediranib (AZD2171), CHIR-258 (dovitinib), CP 673451, CYC 116, E7080, Ki8751, masitinib (AB1010), MGCD-265, motesanib diphosphate (AMG-706), MP-470, OSI-930, Pazopanib Hydrochloride, PD173074, Sorafenib Tosylate (Bay 43-9006), SU 5402, TSU-68 (SU6668), vatalanib, XL880 (GSK1363089, EXEL-2880). Further examples of hedgehog inhibitors include, but are not limited to, vismodegib (2-chloro-N-[4-chloro-3-(2-pyridinyl)phenyl]-4-(methylsulfonyl)-benzamide, GDC-0449); 1-(4-Chloro-3-(trifluoromethyl)phenyl)-3-((3-(4-fluorophenyl)-3,4-dihydro-4-oxo-2-quinazolinyl)methyl)-urea (CAS 330796-24-2); N-[(2S,3R,3′R,3aS,4′aR,6S,6′aR,6′bS,7aR,12′aS,12′bS)-2′,3′,3a,4,4′,4′a,5,5′,6,6′,6′a,6′b,7,7′,7a,8′,10′,12′,12′a,12′b-Eicosahydro-3,6,11′,12′b-tetramethylspiro[furo[3,2-b]pyridine-2(3H),9′(1′H)-naphth[2,1-a]azulen]-3′-yl]-methanesulfonamide (IPI926, CAS 1037210-93-7); and 4-Fluoro-N-methyl-N-[1-[4-(1-methyl-1H-pyrazol-5-yl)-1-phthalazinyl]-4-piperidinyl]-2-(trifluoromethyl)-benzamide (LY2940680, CAS 1258861-20-9); and Erismodegib (LDE225). Selected tyrosine kinase inhibitors are chosen from sunitinib, erlotinib, gefitinib, or sorafenib erlotinib hydrochloride (Tarceva®); linifanib (N-[4-(3-amino-1H-indazol-4-yl)phenyl]-N′-(2-fluoro-5-methylphenyl)urea, also known as ABT 869, avail-able from Genentech); sunitinib malate (Sutent®); bosutinib (4-[(2,4-dichloro-5-methoxyphenyl)amino]-6-methoxy-7-[3-(4-methylpiperazin-1-yl)propoxy]quinoline-3-carbonitrile, also known as SKI-606, described in U.S. Pat. No. 6,780,996); dasatinib (Sprycel®); pazopanib (Votrient®); sorafenib (Nexavar®); zactima (ZD6474); and imatinib or imatinib mesylate (Gil-Vec® and Gleevec®).

In certain embodiments, the cell therapy can be used in combination with a Vascular Endothelial Growth Factor (VEGF) receptor inhibitors, including but not limited to, Bevacizumab (Avastin®), axitinib (Inlyta®); Brivanib alaninate (BMS-582664, (S)—((R)-1-(4-(4-Fluoro-2-methyl-1H-indol-5-yloxy)-5-methylpyrrolo[2,1-f][1,2,4]triazin-6-yloxy)propan-2-yl)2-aminopropanoate); Sorafenib (Nexavar®); Pazopanib (Votrient®); Sunitinib malate (Sutent®); Cediranib (AZD2171, CAS 288383-20-1); Vargatef (BIBF1120, CAS 928326-83-4); Foretinib (GSK1363089); Telatinib (BAY57-9352, CAS 332012-40-5); Apatinib (YN968D1, CAS 811803-05-1); Imatinib (Gleevec®); Ponatinib (AP24534, CAS 943319-70-8); Tivozanib (AV951, CAS 475108-18-0); Regorafenib (BAY73-4506, CAS 755037-03-7); Vatalanib dihydrochloride (PTK787, CAS 212141-51-0); Brivanib (BMS-540215, CAS 649735-46-6); Vandetanib (Caprelsa® or AZD6474); Motesanib diphosphate (AMG706, CAS 857876-30-3, N-(2,3-dihydro-3,3-dimethyl-1H-indol-6-yl)-2-[(4-pyridinylmethyl)amino]-3-pyridinecarboxamide, described in PCT Publication No. WO 02/066470); Dovitinib dilactic acid (TKI258, CAS 852433-84-2); Linfan-ib (ABT869, CAS 796967-16-3); Cabozantinib (XL184, CAS 849217-68-1); Lestaurtinib (CAS 111358-88-4); N-[5-[[[5-(1,1-Dimethylethyl)-2-oxazolyl]methyl]thio]-2-thiazolyl]-4-piperidinecarboxamide (BMS38703, CAS 345627-80-7); (3R,4R)-4-Amino-1((4-((3-methoxyphenyl)amino)pyrrolo[2,1-f][1,2,4]triazin-5-yl)methyl)piperidin-3-ol (BMS690514); N-(3,4-Dichloro-2-fluorophenyl)-6-methoxy-7-[[(3aα,5β,6aα)-octahydro-2-methylcyclopenta[c]pyrrol-5-yl]methoxy]-4-quinazolinamine (XL647, CAS 781613-23-8); 4-Methyl-3-[[1-methyl-6-(3-pyridinyl)-1H-pyrazolo[3,4-d]pyrimidin-4-yl]amino]-N-[3-(trifluoromethyl)phenyl]-benzamide (BHG712, CAS 940310-85-0); and Aflibercept (Eylea®).

In some embodiments, the cell therapy described herein can be used in combination with a PI3K inhibitor. The PI3K inhibitor can be an inhibitor of delta and gamma isoforms of PI3K. Examples of PI3K inhibitors include, but are not limited to, 4-[2-(1H-Indazol-4-yl)-6-[[4-(methylsulfonyl)piperazin-1-yl]methyl]thieno[3,2-d]pyrimidin-4-yl]morpholine; 2-Methyl-2-[4-[3-methyl-2-oxo-8-(quinolin-3-yl)-2,3-dihydroimidazo[4,5-c]quinolin-1-yl]phenyl]propionitrile; 4-(trifluoromethyl)-5-(2,6-dimorpholinopyrimidin-4-yl)pyridin-2-amine; Tozasertib (VX680 or MK-0457, CAS 639089-54-6); (5Z)-5-[[4-(4-Pyridinyl)-6-quinolinyl]methylene]-2,4-thiazolidinedione (GSK1059615, CAS 958852-01-2); (1E,4S,4aR,5R,6aS,9aR)-5-(Acetyloxy)-1-[(di-2-propenylamino)methylene]-4,4a,5,6,6a,8,9,9a-octahydro-11-hydroxy-4-(methoxymethyl)-4a,6a-dimethyl-cyclopenta[5,6]naphtho[1,2-c]pyran-2,7,10(1H)-trione (PX866, CAS 502632-66-8); 8-Phenyl-2-(morpholin-4-yl)-chromen-4-one (LY294002, CAS 154447-36-6); 2-Amino-8-ethyl-4-methyl-6-(1H-pyrazol-5-yl)pyrido[2,3-d]pyrimidin-7(8H)-one (SAR 245409 or XL 765); 1,3-Dihydro-8-(6-methoxy-3-pyridinyl)-3-methyl-1-[4-(1-piperazinyl)-3-(trifluoromethyl)phenyl]-2H-imidazo[4,5-c]quinolin-2-one, (2Z)-2-butenedioate (1:1) (BGT 226); 5-Fluoro-3-phenyl-2-[(1S)-1-(9H-purin-6-ylamino)ethyl]-4(3H)-quinazolinone (CAL101); 2-Amino-N-[3-[N-[3-[(2-chloro-5-methoxyphenyl)amino]quinoxalin-2-yl]sulfamoyl]phenyl]-2-methylpropanamide (SAR 245408 or XL 147); and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (BYL719).

In some embodiments, the cell therapy described herein can be used in combination with a mTOR inhibitor, e.g., one or more mTOR inhibitors chosen from one or more of rapamycin, temsirolimus (TORISEL®), AZD8055, BEZ235, BGT226, XL765, PF-4691502, GDC0980, SF1126, OSI-027, GSK1059615, KU-0063794, WYE-354, Palomid 529 (P529), PF-04691502, or PKI-587. ridaforolimus (formally known as deferolimus, (1R,2R,4S)-4-[(2R)-2 [(1R,9S,12S,15R,16E,18R,19R,21R,23S,24E,26E,28Z,30S,32S,35R)-1,18-dihydroxy-19,30-dimethoxy-15,17,21,23, 29,35-hexamethyl-2,3,10,14,20-pentaoxo-11,36-dioxa-4-azatricyclo[30.3.1.04,9] hexatriaconta-16,24,26,28-tetraen-12-yl]propyl]-2-methoxycyclohexyl di-methylphosphinate, also known as AP23573 and MK8669, and described in PCT Publication No. WO 03/064383); everolimus (Afinitor® or RAD001); rapamycin (AY22989, Sirolimus®); simapi-mod (CAS 164301-51-3); emsirolimus, (5-{2,4-Bis[(3S)-3-methylmorpholin-4-yl]pyrido[2,3-d]pyrimidin-7-yl}-2-methoxyphenyl)methanol (AZD8055); 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4); and N2-[1,4-dioxo-4-[[4-(4-oxo-8-phenyl-4H-1-benzopyran-2-yl)morpholinium-4-yl]methoxy]butyl]-L-arginylglycyl-L-α-aspartylL-serine-, inner salt (SF1126, CAS 936487-67-1), (1r,4r)-4-(4-amino-5-(7-methoxy-1H-indol-2-yl)imidazo[1,5-f][1,2,4]triazin-7-yl)cyclohexanecarboxylic acid (OSI-027); and XL765.

In some embodiments, the cell therapy can be used in combination with a BRAF inhibitor, e.g., GSK2118436, RG7204, PLX4032, GDC-0879, PLX4720, and sorafenib tosylate (Bay 43-9006). In further embodiments, a BRAF inhibitor includes, but is not limited to, regorafenib (BAY73-4506, CAS 755037-03-7); tuvizanib (AV951, CAS 475108-18-0); vemurafenib (Zel-Boraf®, PLX-4032, CAS 918504-65-1); encorafenib (also known as LGX818); 1-Methyl-5-[[2-[5-(trifluoromethyl)-1H-imidazol-2-yl]-4-pyridinyl]oxy]-N-[4-(trifluoromethyl)phenyl-1H-benzimidazol-2-amine (RAF265, CAS 927880-90-8); 5-[1-(2-Hydroxyethyl)-3-(pyridin-4-yl)-1H-pyrazol-4-yl]-2,3-dihydroinden-1-one oxime (GDC-0879, CAS 905281-76-7); 5-[2-[4-[2-(Dimethylamino)ethoxy]phenyl]-5-(4-pyridinyl)-1H-imidazol-4-yl]-2,3-dihydro-1H-Inden-1-one oxime (GSK2118436 or SB590885); (+/−)-Methyl (5-(2-(5-chloro-2-methylphenyl)-1-hydroxy-3-oxo-2,3-dihydro-1H-isoindol-1-yl)-1H-benzimidazol-2-yl)carbamate (also known as XL-281 and BMS908662) and N-(3-(5-chloro-1H-pyrrolo[2,3-b]pyridine-3-carbonyl)-2,4-difluorophenyl)propane-1-sulfonamide (also known as PLX4720).

In some embodiments, the cell therapy described herein can be used in combination with a MEK inhibitor. Any MEK inhibitor can be used in combination including, but not limited to, selumetinib (5-[(4-bromo-2-chlorophenyl)amino]-4-fluoro-N-(2-hydroxyethoxy)-1-methyl-1H-benzimidazole-6-carboxamide, also known as AZD6244 or ARRY 142886); ARRY-142886 trametinib dimethyl sulfoxide (GSK-1120212, CAS 1204531-25-80); G02442104 (also known as GSK1120212), RDEA436; N-[3,4-Difluoro-2-[(2-fluoro-4-iodophenyl)amino]-6-methoxyphenyl]-1-[(2R)-2,3-dihydroxypropyl]-cyclopropanesulfonamide (also known as RDEA119 or BAY869766); RDEA119/BAY 869766, AS703026; G00039805 (also known as AZD-6244 or selumetinib), BIX 02188; BIX 02189; 2-[(2-Chloro-4-iodophenyl)amino]-N-(cyclopropylmethoxy)-3,4-difluoro-benzamide (also known as CI-1040 or PD184352); CI-1040 (PD-184352), N-[(2R)-2,3-Dihydroxypropoxy]-3,4-difluoro-2-[(2-fluoro-4-iodophenyl)amino]-benzamide (also known as PD0325901); PD03259012′-amino-3′-methoxyflavone (also known as PD98059 available from Biaffin GmbH & Co., KG, Germany); PD98059, 2,3-bis[amino[(2-aminophenyl)thio]methylene]-butanedinitrile (also known as U0126); U0126, XL-518 (also known as GDC-0973, Cas No. 1029872-29-4, available from ACC Corp.); GDC-0973 (Methanone, [3,4-difluoro-2-[(2-fluoro-4-iodophenyl)amino]phenyl][3-hydroxy-3-(25)-2-piperidinyl-1-azetidinyl]-), G-38963; and G02443714 (also known as AS703206), or a pharmaceutically acceptable salt or solvate thereof. Further examples of MEK inhibitors include, but are not limited to, benimetinib (6-(4-bromo-2-fluorophenylamino)-7-fluoro-3-methyl-3H-benzoimidazole-5-carboxylic acid (2-hydroxyethyoxy)-amide, also known as MEK162, CAS 1073666-70-2); 2,3-Bis[amino[(2-aminophenyl)thio]methylene]-butanedinitrile (also known as U0126 and described in U.S. Pat. No. 2,779,780); (3S,4R,5Z,8S,9S,11E)-14-(Ethylamino)-8,9,16-trihydroxy-3,4-dimethyl-3,4,9, 19-tetrahydro-1H-2-benzoxacyclotetradecine-1,7(8H)-dione] (also known as E6201); vemurafenib (PLX-4032, CAS 918504-65-1); (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5); pimasertib (AS-703026, CAS 1204531-26-9); 2-(2-Fluoro-4-iodophenylamino)-N-(2-hydroxyethoxy)-1,5-dimethyl-6-oxo-1,6-dihydropyridine-3-carboxamide (AZD 8330); and 3,4-Difluoro-2-[(2-fluoro-4-iodophenyl)amino]-N-(2-hydroxyethoxy)-5-[(3-oxo-[1,2]oxazinan-2-yl)methyl]benzamide (CH 4987655 or Ro 4987655).

In some embodiments, the cell therapy described herein can be used in combination with a JAK2 inhibitor, e.g., CEP-701, INCB18424, CP-690550 (tasocitinib). Example JAK inhibitors include, but are not limited to, ruxolitinib (Jakafi®); tofacitinib (CP690550); axitinib (AG013736, CAS 319460-85-0); 5-Chloro-N2-[(1S)-1-(5-fluoro-2-pyrimidinyl)ethyl]-N4-(5-methyl-1H-pyrazol-3-y)-12,4-pyrimidinediamine (AZD1480, CAS 935666-88-9); (9E)-15-[2-(1-Pyrrolidinyl)ethoxy]-7,12,26-trioxa-19,21,24-triazatetracyclo[18.3.1.12,5.114,18]-hexacosa-1(24),2,4,9,14,16,18(25),20,22-nonaene (SB-1578, CAS 937273-04-6); momelotinib (CYT 387); baricitinib (INCB-028050 or LY-3009104); pacritinib (SB1518); (16E)-14-Methyl-20-oxa-5,7,14,27-tetraazatetracyclo[19.3.1.12,6.18,12]heptacosa-1(25),2,4,6(27),8,10,12(26),16,21,23-decaene (SB 1317); gandotinib (LY 2784544); and N,N-cicyclopropyl-4-[(1,5-dimethyl-1H-pyrazol-3-yl)amino]-6-ethyl-1,6-dihydro-1-methyl-imidazo[4,5-d]pyrrolo[2,3-b]pyridine-7-carboxamide (BMS 911543).

In some embodiments, the combination therapies disclosed herein include paclitaxel or a paclitaxel agent, e.g., TAXOL®, protein-bound paclitaxel (e.g., ABRAXANE®). Exemplary paclitaxel agents include, but are not limited to, nanoparticle albumin-bound paclitaxel (ABRAX-ANE, marketed by Abraxis Bioscience), docosahexaenoic acid bound-paclitaxel (DHA-paclitaxel, Taxoprexin, marketed by Protarga), polyglutamate bound-paclitaxel (PG-paclitaxel, paclitaxel poliglumex, CT-2103, XYOTAX, marketed by Cell Therapeutic), the tumor-activated prodrug (TAP), ANG105 (Angiopep-2 bound to three molecules of paclitaxel, marketed by ImmunoGen), paclitaxel-EC-1 (paclitaxel bound to the erbB2-recognizing peptide EC-1; see Li et al., Biopolymers (2007) 87:225-230), and glucose-conjugated paclitaxel (e.g., 2′-paclitaxel methyl 2-glucopyranosyl succinate).

### Methods

The present disclosure provides methods for generating an engineered cell. In some aspects, the method can comprise (a) delivering a nucleic acid molecule expressing a chimeric polypeptide into a cell; and (b) expressing the nucleic acid molecule in the cell, thereby generating the engineered cell. The chimeric polypeptide can be a chimeric antigen receptor as described herein.

The present disclosure also provides methods for administering an engineered cell as described herein. The engineered cell can be an engineered immune cell. The engineered immune cell can be a T cell. The engineered immune cell can be derived from an autologous T cell. The engineered immune cell can be derived from an allogeneic T cell.

In some aspects, provided herein is a method for administering an engineered immune cell comprising a chimeric polypeptide comprising (i) an enhancer moiety capable of enhancing one or more activities of the engineered immune cell, and (ii) an inducible cell death moiety capable of effecting death of the engineered immune cell upon contacting the chimeric polypeptide with a cell death activator. The enhancer moiety can be linked to the inducible cell death moiety. The engineered immune cell can further comprise one or more chimeric antigen receptors (CARs) comprising a binding moiety. The binding moiety can comprise a first antigen binding domain, which first antigen binding domain suppresses or reduces a subject's immune response toward the engineered immune cell when administered into the subject. The binding moiety can further comprise a second antigen binding domain capable of binding to a disease-associated antigen. An individual CAR of the one or more CARs can comprise (i) the first antigen binding domain, (ii) the second antigen binding domain, or (iii) both the first antigen binding domain and the second antigen binding domain. Each CAR of the one or more CARs can further comprise a transmembrane domain and an intracellular signaling domain.

In some aspects, provided herein is a method of administering an engineered immune cell comprising one or more chimeric antigen receptors (CARs) comprising a binding moiety. The binding moiety can comprise a first antigen binding domain capable of binding to an immune cell antigen and a second antigen binding domain capable of binding to a disease-associated antigen. Each CAR of the one or more CARs can further comprise a transmembrane domain and an intracellular signaling domain. The engineered immune cell can further comprise an enhancer moiety capable of enhancing one or more activities of the engineered immune cell. In some cases, an endogenous T cell receptor (TCR) of the engineered immune cell can be inactivated. The engineered immune cell can exhibit (i) enhanced degree of persistence by remaining viable in vitro for at least about 5, 10, 15, 20, 25, 30, 35, 40, 45, 50, 55, 60, 65, 70, 75, 80, 85, 90, 95, 100, 150, 200, 250, 300, 350, 400, 450, 500, or more days while in presence of cells (e.g., cancer cells, immune cells, or both) that are heterologous to the engineered immune cell, (ii) enhanced degree of expansion by at least about 2-fold, 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 110-fold, 120-fold, 130-fold, 140-fold, 150-fold, 200-fold, 250-fold, 300-fold, or more within 15 days, or (iii) enhanced cytotoxicity against a target cell comprising the immune cell antigen such as CD2, CD3, CD5 or CD7, or the disease-associated antigen CD19, compared to an additional engineered immune cell comprising the one or more CARs but not the enhancer moiety. In some cases, the engineered immune cell can exhibit enhanced degree of expansion by at least about 50-fold, 60-fold, 70-fold, 80-fold, 90-fold, 100-fold, 110-fold, 120-fold, 130-fold, 140-fold, 150-fold, 160-fold, 170-fold, 180-fold, 190-fold, 200-fold, 210-fold, 220-fold, 230-fold, 240-fold, 250-fold, 260-fold, 270-fold, 280-fold, 290-fold, 300-fold, 350-fold, 400-fold, 450-fold, 500-fold, or more within 30 days. In some cases, the engineered immune cell can exhibit enhanced degree of expansion by at least about 100-fold, 200-fold, 300-fold, 400-fold, 500-fold, 600-fold, 700-fold, 800-fold, 900-fold, 1,000-fold, 2,000-fold, 3,000-fold, 4,000-fold, 5,000-fold, 6,000-fold, 7,000-fold, 8,000-fold, 9,000-fold, 10,000-fold, 20,000-fold, 30,000-fold, 40,000-fold, 50,000-fold, 60,000-fold, 70,000-fold, 80,000-fold, 90,000-fold, 100,000-fold, 200,000-fold, 300,000-fold, 400,000-fold, 500,000-fold, 600,000-fold, 700,000-fold, 800,000-fold, 900,000-fold, 1,000,000-fold, or more within 60 days. The viability or expansion can be measured in the presence of stimulation, for example, stimulation by a cancer antigen or a cancer cell. The viability or expansion can be measured in the presence of multiple rounds or repeated stimulations.

In some aspects, provided herein is a method of administering a cell (e.g., an engineered immune cell), comprising a functionally inactive T cell receptor (TCR). The cell can further comprise one or more chimeric antigen receptors (CARs). Each individual CAR of the one or more CARs can comprise a binding moiety. The binding moiety can comprise (i) a first antigen binding domain, which first antigen binding domain suppresses or reduces a subject's immune response toward the engineered immune cell when administered into the subject and (ii) a second antigen binding domain that binds to a disease-associated antigen such as CD19. Each CAR of the one or more CARs can further comprise a transmembrane domain and an intracellular signaling domain.

In some aspects, provided herein is a method of administering an engineered immune cell comprising an enhancer moiety capable of enhancing one or more activities of the engineered immune cell. The engineered cell can further comprise a chimeric antigen receptor (CAR) comprising an antigen binding domain that specifically binds CD7. The CAR can further comprise a transmembrane domain and an intracellular signaling domain. The endogenous CD7 in the engineered immune cell can be inactivated. In some embodiments, the engineered cell can comprise a CAR comprising an antigen binding domain that specifically binds an immune cell antigen. The immune cell antigen can be any immune cell antigen described herein such as CD2, CD3 or CD5. The endogenous immune cell antigen of the engineered cell, which the antigen binding domain binds, can be inactivated in the engineered cell.

In some aspects, provided herein is a method of administering an engineered immune cell comprising a single chimeric antigen receptor (CAR) comprising (i) a first antigen binding domain that specifically binds CD7 and (ii) a second antigen binding domain capable of binding to a disease-associated antigen. The CAR can further comprise a transmembrane domain and an intracellular signaling domain. A gene encoding endogenous CD7 can be inactivated in the engineered immune cell.

In some aspects, provided herein is a method of administering an engineered immune cell comprising a single chimeric antigen receptor (CAR) comprising (i) a first antigen binding domain that specifically binds CD2 and (ii) a second antigen binding domain capable of binding to a disease-associated antigen. The CAR can further comprise a transmembrane domain and an intracellular signaling domain. A gene encoding endogenous CD2 can be inactivated in the engineered immune cell.

In some aspects, provided herein is a method of administering an engineered immune cell comprising a single chimeric antigen receptor (CAR) comprising (i) a first antigen binding domain that specifically binds CD3 and (ii) a second antigen binding domain capable of binding to a disease-associated antigen. The CAR can further comprise a transmembrane domain and an intracellular signaling domain. A gene encoding endogenous CD3 can be inactivated in the engineered immune cell.

In some aspects, provided herein is a method of administering an engineered immune cell comprising a single chimeric antigen receptor (CAR) comprising (i) a first antigen binding domain that specifically binds CD5 and (ii) a second antigen binding domain capable of binding to a disease-associated antigen. The CAR can further comprise a transmembrane domain and an intracellular signaling domain. A gene encoding endogenous CD5 can be inactivated in the engineered immune cell.

The present disclosure also provides methods of treating or diagnosing a disease in a subject. In some cases, the method comprises administering a pharmaceutical composition comprising an engineered immune cell into a subject. The engineered immune cell in the pharmaceutical composition can be derived from an allogeneic immune cell. The engineered immune cell derived from the allogeneic immune cell may not induce graft versus host disease (GVHD) in the subject. The engineered immune cell in the pharmaceutical composition can be derived from an autologous immune cell. In some cases, an endogenous TCR of the engineered immune cell in the pharmaceutical composition is functionally inactive. The engineered immune cell can reduce GVHD in the subject compared to an immune cell having a functionally active TCR. The disease can be a cancer. For example, the cancer can be lymphoma or leukemia.

The present disclosure also provides a method of delivering an allogeneic cell therapy comprising administering to a subject in need thereof a population of engineered immune cells. An individual engineered immune cell of the population can comprise one or more chimeric antigen receptors (CARs) comprising a binding moiety. The binding moiety can comprise a first antigen binding domain capable of binding to an immune cell antigen. The binding moiety can further comprise a second antigen binding domain capable of binding to a disease-associated antigen such as CD19. The first antigen binding domain can suppress or reduce a subject's immune response toward the engineered immune cell when administered into the subject. The engineered immune cell can further comprise an enhancer moiety capable of enhancing one or more activities of the engineered immune cell. The endogenous T cell receptor (TCR) of the engineered immune cell can be inactivated. For example, a gene encoding a subunit of TCR can be inactivated. Various gene editing methods described herein can be used to inactivate endogenous TCRs of a T cell.

In some embodiments, a method provided herein can include activation of a population of cells. In some cases, the cell used to prepare the engineered immune cell can be activated before preparing the engineered immune cell. In some cases, the engineered immune cell can be activated. Activation as used herein can refer to a process whereby a cell transitions from a resting state to an active state. This process can comprise a response to an antigen, migration, and/or a phenotypic or genetic change to a functionally active state. In some aspects, activation can refer to the stepwise process of T cell activation. In some cases, a T cell can require one or more signals to become activated. For example, a T cell can require at least two signals to become fully activated. The first signal can occur after engagement of a TCR by the antigen-MHC complex, and the second signal can occur by engagement of co-stimulatory molecules. Anti-CD3 antibody (or a functional variant thereof) can mimic the first signal and anti-CD28 antibody (or a functional variant thereof) can mimic the second signal in vitro.

In some aspects, a method provided herein can comprise activation of a population of cells. Activation can be performed by contacting a population of cells with a surface having attached thereto an agent that can stimulate a CD3 TCR complex associated signal and a ligand that can stimulate a co-stimulatory molecule on the surface of the cells. In particular, T cell populations can be stimulated in vitro such as by contact with an anti-CD3 antibody or antigen-binding fragment thereof, or an anti-CD2 antibody immobilized on a surface, or by contact with a protein kinase C activator (e.g., bryostatin) sometimes in conjunction with a calcium ionophore. For co-stimulation of an accessory molecule on the surface of the T cells, a ligand that binds the accessory molecule can be used. For example, a population of cells can be contacted with an anti-CD3 antibody and an anti-CD28 antibody, under conditions that can stimulate proliferation of the T cells. In some cases, 4-1BB can be used to stimulate cells. For example, cells can be stimulated with 4-1BB and IL-21 or another cytokine. For activation of either CD4 T cells or CD8 T cells, an anti-CD3 antibody and an anti-CD28 antibody can be used. For example, the agents providing a signal may be in solution or conjugated to a solid phase surface. The ratio of particles to cells may depend on particle size relative to the target cell. In further embodiments, the cells, such as T cells, can be combined with agent-coated beads, where the beads and the cells can be subsequently separated, and optionally cultured. Each bead can be coated with either anti-CD3 antibody or an anti-CD28 antibody, or in some cases, a combination of the two. In an alternative embodiment, prior to culture, the agent-coated beads and cells are not separated but are cultured together. Cell surface proteins may be conjugated by allowing paramagnetic beads to which anti-CD3 antibody and anti-CD28 antibody can be attached (3×28 beads) to contact the T cells. In one embodiment the cells and beads (for example, DYNABEADS® M-450 CD3/CD28 T paramagnetic beads at a ratio of 1:1) are combined in a buffer, for example, phosphate buffered saline (PBS) (e.g., without divalent cations such as, calcium and magnesium). Any cell concentration may be used. The mixture may be cultured for or for about several hours (e.g., about 3 hours) to or to about 14 days or any hourly integer value in between. In another embodiment, the mixture may be cultured for or for about 21 days or for up to or for up to about 21 days. Conditions appropriate for T cell culture can include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, IL-4, IL-7, GM-CSF, IL-10, IL-21, IL-15, TGF beta, and TNF alpha or any other additives for the growth of cells. Other additives for the growth of cells include, but are not limited to, surfactant, plasmanate, and reducing agents such as N-acetyl-cysteine and 2-mercaptoethanol. Media can include RPMI 1640, A1 M-V, DMEM, MEM, α-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, can be included only in experimental cultures, possibly not in cultures of cells that are to be infused into a subject. The target cells can be maintained under conditions necessary to support growth; for example, an appropriate temperature (e.g., 37° C.) and atmosphere (e.g., air plus 5% CO₂). In some instances, T cells that have been exposed to varied stimulation times may exhibit different characteristics. In some cases, a soluble monospecific tetrameric antibody against human CD3, CD28, CD2, or any combination thereof may be used. In some embodiments, activation can utilize an activation moiety, a costimulatory agent, and any combination thereof. In some aspects, an activation moiety binds: a CD3/T cell receptor complex and/or provides costimulation. In some aspects, an activation moiety is any one of anti-CD3 antibody and/or anti-CD28 antibody. In some aspects, a solid phase is at least one of a bead, plate, and/or matrix. In some aspects, a solid phase is a bead. Alternatively or in addition to, the activation moiety may be not be conjugated a substrate, e.g., the activation moiety may be free-floating in a medium.

In some cases, a population of cells can be activated or expanded by co-culturing with tissue or cells. A cell can be an antigen presenting cell. An artificial antigen presenting cells (aAPCs) can express ligands for T cell receptor and costimulatory molecules and can activate and expand T cells for transfer, while improving their potency and function in some cases. An aAPC can be engineered to express any gene for T cell activation. An aAPC can be engineered to express any gene for T cell expansion. An aAPC can be a bead, a cell, a protein, an antibody, a cytokine, or any combination. An aAPC can deliver signals to a cell population that may undergo genomic transplant. For example, an aAPC can deliver a signal 1, signal, 2, signal 3 or any combination. A signal 1 can be an antigen recognition signal. For example, signal 1 can be ligation of a TCR by a peptide-MHC complex or binding of agonistic antibodies directed towards CD3 that can lead to activation of the CD3 signal-transduction complex. Signal 2 can be a co-stimulatory signal. For example, a co-stimulatory signal can be anti-CD28, inducible co-stimulator (ICOS), CD27, and 4-1BB (CD137), which bind to ICOS-L, CD70, and 4-1BBL, respectively. Signal 3 can be a cytokine signal. A cytokine can be any cytokine. A cytokine can be IL-2, IL-7, IL-12, IL-15, IL-21, or any combination thereof. In some cases an artificial antigen presenting cell (aAPC) may be used to activate and/or expand a cell population. In some cases, an artificial may not induce allospecificity. An aAPC may not express HLA in some cases. An aAPC may be genetically modified to stably express genes that can be used to activation and/or stimulation. In some cases, a K562 cell may be used for activation. A K562 cell may also be used for expansion. A K562 cell can be a human erythroleukemic cell line. A K562 cell may be engineered to express genes of interest. K562 cells may not endogenously express HLA class I, IL, or CD1d molecules but may express ICAM-1 (CD54) and LFA-3 (CD58). K562 may be engineered to deliver a signal 1 to T cells. For example, K562 cells may be engineered to express HLA class I. In some cases, K562 cells may be engineered to express additional molecules such as B7, CD80, CD83, CD86, CD32, CD64, 4-1BBL, anti-CD3, anti-CD3 mAb, anti-CD28, anti-CD28mAb, CD1d, anti-CD2, membrane-bound IL-15, membrane-bound IL-17, membrane-bound IL-21, membrane-bound IL-2, truncated CD19, or any combination. In some cases, an engineered K562 cell can expresses a membranous form of anti-CD3 mAb, clone OKT3, in addition to CD80 and CD83. In some cases, an engineered K562 cell can expresses a membranous form of anti-CD3 mAb, clone OKT3, membranous form of anti-CD28 mAb in addition to CD80 and CD83.

An aAPC can be a bead. A spherical polystyrene bead can be coated with antibodies against CD3 and CD28 and be used for T cell activation. A bead can be of any size. In some cases, a bead can be or can be about 3 and 6 micrometers. A bead can be or can be about 4.5 micrometers in size. A bead can be utilized at any cell to bead ratio. For example, a 3 to 1 bead to cell ratio at 1 million cells per milliliter can be used. An aAPC can also be a rigid spherical particle, a polystyrene latex microbeads, a magnetic nano- or micro-particles, a nanosized quantum dot, a 4, poly(lactic-co-glycolic acid) (PLGA) microsphere, a nonspherical particle, a 5, carbon nanotube bundle, a 6, ellipsoid PLGA microparticle, a 7, nanoworms, a fluidic lipid bilayer-containing system, an 8, 2D-supported lipid bilayer (2D-SLBs), a 9, liposome, a 10, RAFTsomes/microdomain liposome, an 11, SLB particle, or any combination thereof. In some cases, an aAPC can expand CD4 T cells. For example, an aAPC can be engineered to mimic an antigen processing and presentation pathway of HLA class II-restricted CD4 T cells. A K562 can be engineered to express HLA-D, DP α, DP β chains, Ii, DM α, DM β, CD80, CD83, or any combination thereof. For example, engineered K562 cells can be pulsed with an HLA-restricted peptide in order to expand HLA-restricted antigen-specific CD4 T cells. In some cases, the use of aAPCs can be combined with exogenously introduced cytokines for T cell activation, expansion, or any combination. Cells can also be expanded in vivo, for example in the subject's blood after administration of genomically transplanted cells into a subject.

In some embodiments, a method provided herein can comprise transduction of a population of cells. In some embodiments, a method comprises introducing a polynucleotide encoding for a cellular receptor such as a chimeric antigen receptor and/or a T cell receptor. In some cases, a transfection of a cell can be performed.

In some embodiments, a viral supernatant comprising a polynucleotide encoding for a cellular receptor such as a CAR and/or TCR is generated. In some embodiments, a viral vector can be a retroviral vector, a lentiviral vector and/or an adeno-associated viral vector. Packaging cells can be used to form virus particles capable of infecting a host cell. Such cells can include 293 cells, (e.g., for packaging adenovirus), and Psi2 cells or PA317 cells (e.g., for packaging retrovirus). Viral vectors can be generated by producing a cell line that packages a nucleic acid vector into a viral particle. The vectors can contain the minimal viral sequences required for packaging and subsequent integration into a host. The vectors can contain other viral sequences being replaced by an expression cassette for the polynucleotide(s) to be expressed. The missing viral functions can be supplied in trans by the packaging cell line. For example, AAV vectors can comprise ITR sequences from the AAV genome which are required for packaging and integration into the host genome. Viral DNA can be packaged in a cell line, which can contain a helper plasmid encoding the other AAV genes, namely rep and cap, while lacking ITR sequences. The cell line can also be infected with adenovirus as a helper. The helper virus can promote replication of the AAV vector and expression of AAV genes from the helper plasmid. Contamination with adenovirus can be reduced by, e.g., heat treatment to which adenovirus is more sensitive than AAV. Additional methods for the delivery of nucleic acids to cells can be used, for example, as described in US20030087817, incorporated herein by reference.

In some embodiments, a host cell can be transiently or non-transiently transfected with one or more vectors described herein. A cell can be transfected as it naturally occurs in a subject. A cell can be taken or derived from a subject and transfected. A cell can be derived from cells taken from a subject, such as a cell line. In some embodiments, a cell transfected with one or more vectors described herein is used to establish a new cell line comprising one or more vector-derived sequences. Non-limiting examples of vectors for eukaryotic host cells include but are not limited to: pBs, pQE-9 (Qiagen), phagescript, PsiX174, pBluescript SK, pBsKS, pNH8a, pNH16a, pNH18a, pNH46a (Stratagene); pTrc99A, pKK223-3, pKK233-3, pDR540, pRITS (Pharmacia). Eukaryotic: pWL-neo, pSv2cat, pOG44, pXTI, pSG (Stratagene) pSVK3, pBPv, pMSG, pSVL (Pharmacia). Also, any other plasmids and vectors can be used as long as they are replicable and viable in a selected host. Any vector and those commercially available (and variants or derivatives thereof) can be engineered to include one or more recombination sites for use in the methods. Such vectors can be obtained from, for example, Vector Laboratories Inc., Invitrogen, Promega, Novagen, NEB, Clontech, Boehringer Mannheim, Pharmacia, EpiCenter, OriGenes Technologies Inc., Stratagene, PerkinElmer, Pharmingen, and Research Genetics. Other vectors of interest include eukaryotic expression vectors such as pFastBac, pFastBacHT, pFastBacDUAL, pSFV, and pTet-Splice (Invitrogen), pEUK-C1, pPUR, pMAM, pMAMneo, pBI101, pBI121, pDR2, pCMVEBNA, and pYACneo (Clontech), pSVK3, pSVL, pMSG, pCH110, and pKK232-8 (Pharmacia, Inc.), p3′SS, pXTI, pSG5, pPbac, pMbac, pMClneo, and pOG44 (Stratagene, Inc.), and pYES2, pAC360, pBlueBa-cHis A, B, and C, pVL1392, pBlueBac111, pCDM8, pcDNA1, pZeoSV, pcDNA3 pREP4, pCEP4, and pEBVHis (Invitrogen, Corp.), and variants or derivatives thereof. Other vectors include pUC18, pUC19, pBlueScript, pSPORT, cosmids, phagemids, YAC's (yeast artificial chromosomes), BAC's (bacterial artificial chromosomes), P1 (Escherichia coli phage), pQE70, pQE60, pQE9 (quagan), pBS vectors, PhageScript vectors, BlueScript vectors, pNH8A, pNH16A, pNH18A, pNH46A (Stratagene), pcDNA3 (Invitrogen), pGEX, pTrsfus, pTrc99A, pET-5, pET-9, pKK223-3, pKK233-3, pDR540, pRITS (Pharmacia), pSPORT1, pSPORT2, pCMVSPORT2.0 and pSYSPORT1 (Invitrogen) and variants or derivatives thereof. Additional vectors of interest can also include pTrxFus, pThioHis, pLEX, pTrcHis, pTrcHis2, pRSET, pBlueBa-cHis2, pcDNA3.1/His, pcDNA3.1(−)/Myc-His, pSecTag, pEBVHis, pPIC9K, pPIC3.5K, pA081S, pPICZ, pPICZA, pPICZB, pPICZC, pGAPZA, pGAPZB, pGAPZC, pBlue-Bac4.5, pBlueBacHis2, pMelBac, pSinRep5, pSinHis, pIND, pIND(SP1), pVgRXR, pcDNA2.1, pYES2, pZEr01.1, pZErO-2.1, pCR-Blunt, pSE280, pSE380, pSE420, pVL1392, pVL1393, pCDM8, pcDNA1.1, pcDNA1.1/Amp, pcDNA3.1, pcDNA3.1/Zeo, pSe, SV2, pRc/CMV2, pRc/RSV, pREP4, pREP7, pREP8, pREP9, pREP 10, pCEP4, pEBVHis, pCR3.1, pCR2.1, pCR3.1-Uni, and pCRBac from Invitrogen; X ExCell, X gt11, pTrc99A, pKK223-3, pGEX-1X T, pGEX-2T, pGEX-2TK, pGEX-4T-1, pGEX-4T-2, pGEX-4T-3, pGEX-3X, pGEX-5X-1, pGEX-5X-2, pGEX-5X-3, pEZZ18, pRIT2T, pMC1871, pSVK3, pSVL, pMSG, pCH110, pKK232-8, pSL1180, pNEO, and pUC4K from Pharmacia; pSCREEN-1b(+), pT7Blue(R), pT7Blue-2, pCITE-4-abc(+), pOCUS-2, pTAg, pET-32L1C, pET-30LIC, pBAC-2 cp LIC, pBACgus-2 cp LIC, pT7Blue-2 LIC, pT7Blue-2, X SCREEN-1, X BlueSTAR, pET-3abcd, pET-7abc, pET9abcd, pETI1 abcd, pETI2abc, pET-14b, pET-15b, pET-16b, pET-17b-pET-17xb, pET-19b, pET-20b(+), pET-21abcd(+), pET-22b(+), pET-23abcd(+), pET-24abcd (+), pET-25b(+), pET-26b(+), pET-27b(+), pET-28abc(+), pET-29abc(+), pET-30abc(+), pET-31b(+), pET-32abc(+), pET-33b(+), pBAC-1, pBACgus-1, pBAC4x-1, pBACgus4x-1, pBAC-3 cp, pBACgus-2 cp, pBACsurf-1, pig, Signal pig, pYX, Selecta Vecta-Neo, Selecta Vecta-Hyg, and Selecta Vecta-Gpt from Novagen; pLexA, pB42AD, pGBT9, pAS2-1, pGAD424, pACT2, pGAD GL, pGAD GH, pGAD10, pGilda, pEZM3, pEGFP, pEGFP-1, pEGFPN, pEGFP-C, pEBFP, pGFPuv, pGFP, p6×His-GFP, pSEAP2-Basic, pSEAP2-Contral, pSEAP2-Promoter, pSEAP2-Enhancer, p I3 gal-Basic, pl3 gal-Control, p I3 gal-Promoter, p I3 gal-Enhancer, pCMV, pTet-Off, pTet-On, pTK-Hyg, pRetro-Off, pRetro-On, pIRESlneo, pIRESihyg, pLXSN, pLNCX, pLAPSN, pMAMneo, pMAMneo-CAT, pMAMneo-LUC, pPUR, pSV2neo, pYEX4T-1/2/3, pYEX-S1, pBacPAK-His, pBacPAK8/9, pAcUW31, BacPAK6, pTriplEx, 2Xgt10, Xgt11, pWE15, and X TriplEx from Clontech; Lambda ZAP II, pBK-CMV, pBK-RSV, pBluescript II KS+/−, pBluescript II SK+/−, pAD-GAL4, pBD-GAL4 Cam, pSurfscript, Lambda FIX II, Lambda DASH, Lambda EMBL3, Lambda EMBL4, SuperCos, pCR-Scrigt Amp, pCR-Script Cam, pCR-Script Direct, pBS+/−, pBC KS+/−, pBC SK+/−, Phag-escript, pCAL-n-EK, pCAL-n, pCAL-c, pCAL-kc, pET-3abcd, pET-llabcd, pSPUTK, pESP-1, pCMVLacI, pOPRSVI/MCS, pOPI3 CAT, pXT1, pSG5, pPbac, pMbac, pMClneo, pMClneo Poly A, pOG44, pOG45, pFRTI3GAL, pNE0I3GAL, pRS403, pRS404, pRS405, pRS406, pRS413, pRS414, pRS415, and pRS416 from Stratagene, pPC86, pDBLeu, pDBTrp, pPC97, p2.5, pGAD1-3, pGAD10, pACt, pACT2, pGADGL, pGADGH, pAS2-1, pGAD424, pGBT8, pGBT9, pGAD-GAL4, pLexA, pBD-GAL4, pHISi, pHISi-1, placZi, pB42AD, pDG202, pJK202, pJG4-5, pNLexA, pYESTrp, and variants or derivatives thereof. In some embodiments, a vector can be a minicircle vector. A vector provided herein can be used to deliver a polypeptide coding for a CAR and/or TCR.

Transduction and/or transfection can be performed by any one of: non-viral transfection, biolistics, chemical transfection, electroporation, nucleofection, heat-shock transfection, lipofection, microinjection, or viral transfection. In some embodiments a provided method comprises viral transduction, and the viral transduction comprises a lentivirus. Viral particles can be used to deliver a viral vector comprising a polypeptide sequence coding for a cellular receptor into a cell ex vivo or in vivo. In some cases, a viral vector as disclosed herein may be measured as pfu (plaque forming units). In some cases, the pfu of recombinant virus or viral vector of the compositions and methods of the disclosure may be about 10⁸ to about 5×10¹⁰ pfu. In some cases, recombinant viruses of this disclosure are at least about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, and 5×10¹⁰ pfu. In some cases, recombinant viruses of this disclosure are at most about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, and 5×10¹⁰ pfu. In some aspects, the viral vector of the disclosure may be measured as vector genomes. In some cases, recombinant viruses of this disclosure are 1×10¹⁰ to 3×10¹² vector genomes, or 1×10⁹ to 3×10³ vector genomes, or 1×10⁸ to 3×10¹⁴ vector genomes, or at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹³, 1×10¹⁶, 1×10¹⁷, and 1×10¹⁸ vector genomes, or are 1×10⁸ to 3×10¹⁴ vector genomes, or are at most about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ vector genomes. In some cases, a viral vector provided herein can be measured using multiplicity of infection (MOI). In some cases, MOI may refer to the ratio, or multiple of vector or viral genomes to the cells to which the nucleic may be delivered. In some cases, the MOI may be 1×10⁶. In some cases, the MOI may be 1×10⁵ to 1×10⁷. In some cases, the MOI may be 1×10⁴ to 1×10⁸. In some cases, recombinant viruses of the disclosure are at least about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI. In some cases, recombinant viruses of this disclosure are 1×10⁸ to 3×10¹⁴ MOI, or are at most about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, and 1×10¹⁸ MOI. In some cases, a viral vector is introduced at a multiplicity of infection (MOI) from about 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶ 4×10⁶, 5×10⁶, 6×10⁶, 7×10⁶, 8×10⁶, 9×10⁶, 1×10⁷, 2×10⁷, 3×10⁷, or up to about 9×10⁹ genome copies/virus particles per cell.

The transfection efficiency of cells with any of the nucleic acid delivery platforms described herein, for example, transduction, can be or can be about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9%. In some embodiments, a method can comprise adding an infective agent to a composition comprising a population of cells. An infective agent can comprise polybrene. In some aspects, an infective agent can enhance efficiency of viral infection. An infective agent can enhance viral infectivity from about 100 to 1,000 fold. Polybrene can be added to a composition at a concentration from about 5 ug to 10 ug per ml.

In some embodiments, a method provided herein can comprise a non-viral approach of introducing a cellular receptor to a cell. Non-viral approaches can include but are not limited to: CRISPR associated proteins (Cas proteins, e.g., Cas9), Zinc finger nuclease (ZFN), Transcription Activator-Like Effector Nuclease (TALEN), Argonaute nucleases, and meganucleases. Nucleases can be naturally existing nucleases, genetically modified, and/or recombinant. Non-viral approaches can also be performed using a transposon-based system (e.g. PiggyBac, Sleeping beauty).

In some embodiments, a method provided herein can utilize a PiggyBac system to introduce an exogenous polypeptide to a cell. A PiggyBac system comprises two components, a transposon and a transposase. The PiggyBac transposase facilitates the integration of the transposon specifically at ‘TTAA’ sites randomly dispersed in the genome. The predicted frequency of ‘TTAA’ in the genome is approximately 1 in every 256 base-pairs of DNA sequence. Unlike other transposons, the PB transposase also enables the excision of the transposon in a completely seamless manner, leaving no sequences or mutations behind. Furthermore, PiggyBac offers a large cargo-carrying capacity (over 200 kb has been demonstrated) with no known upper limit. PB performance levels can be increased by codon-optimization strategies, mutations, deletions, additions, substitutions, and any combination thereof. In some cases, PB can have a larger cargo (approximately 9.1-14.3 kb), a higher transposition activity, and its footprint-free characteristic can make it appealing as a gene editing tool. In some aspects, PB can comprise a few features: high efficiency transposition; large cargo; steady long-term expression; the trans-gene is integrated as a single copy; tracking the target gene in vivo by a noninvasive mark instead of traditional method such as PCR; easy to determine the integration site, and combinations thereof.

In some aspects, a method provided herein can utilize a Sleeping Beauty (SB) System to introduce a polypeptide coding for a cellular receptor to a cell. SB was engineered from ancient Tc1/mariner transposon fossils found within the Salmonid genomes by in vitro evolution. The SB ITRs (230 bp) contain imperfect direct repeats (DRs) of 32 bp in length that can serve as recognition signals for the transposase. Binding affinity and spacing between the DR elements within ITR has involved in transpositional activities. The SB transposase can be a 39 kDa protein that possess DNA binding polypeptide, a nuclear localization signal (NLS) and the catalytic domain, featured by a conserved amino acid motif (DDE). Various screens mutagenizing the primary amino acid sequence of the SB transposase resulted in hyperactive transposase versions. In some cases, a modified SB can be utilized. Modified SBs can contain mutations, deletions and additions within ITRs of the original SB transposon. Modified SBs can comprise: pT2, pT3, pT2B, pT4, SB100X, and combinations thereof. Non-limited examples of modified SBs can be selected from: SB10, SB11 (3-fold higher than SB10), SB12 (4-fold higher than SB10), HSB1-HSB5 (up to 10-fold higher than SB10), HSB13-HSB17 (HSB17 is 17-fold higher than SB10), SB100× (100-fold higher than SB10), SB150× (130-fold higher than SB10), and any combination thereof. In some cases, SB100× is 100-fold hyperactive compared to the originally resurrected transposase (SB10). In some aspects, SB transposition excision leaves a footprint (3 bp) at the cargo site. Integration occurs into TA dinucleotides of the genome, and results in target site duplications, generated by the host repair machinery. In some cases, SB appears to possess a nearly unbiased, close-to-random integration profile. Transposon integration can be artificially targeted (˜10%) to a predetermined genomic locus in wildtype systems, however in chimeric systems provided herein, SB transposon integration can be directed to a predetermined locus with efficiencies over 10%.

In some aspects, a non-viral approach may be taken to introduce an exogenous polynucleic acid to a population of cells. In some aspects, a non-viral vector or nucleic acid may be delivered without the use of a virus and may be measured according to the quantity of nucleic acid. Generally, any suitable amount of nucleic acid can be used with the compositions and methods of this disclosure. In some cases, nucleic acid may be at least about 1 pg, 10 pg, 100 pg, 1 pg, 10 pg, 100 pg, 200 pg, 300 pg, 400 pg, 500 pg, 600 pg, 700 pg, 800 pg, 900 pg, 1 μg, 10 μg, 100 μg, 200 μg, 300 μg, 400 μg, 500 μg, 600 μg, 700 μg, 800 μg, 900 μg, 1 ng, 10 ng, 100 ng, 200 ng, 300 ng, 400 ng, 500 ng, 600 ng, 700 ng, 800 ng, 900 ng, 1 mg, 10 mg, 100 mg, 200 mg, 300 mg, 400 mg, 500 mg, 600 mg, 700 mg, 800 mg, 900 mg, 1 g, 2 g, 3 g, 4 g, or 5 g. In some cases, nucleic acid may be at most about 1 μg, 10 μg, 100 μg, 1 μg, 10 μg, 100 μg, 200 μg, 300 μg, 400 μg, 500 μg, 600 μg, 700 μg, 800 μg, 900 μg, 1 g, 10 μg, 100 μg, 200 μg, 300 μg, 400 μg, 500 μg, 600 μg, 700 μg, 800 μg, 900 μg, 1 ng, 10 ng, 100 ng, 200 ng, 300 ng, 400 ng, 500 ng, 600 ng, 700 ng, 800 ng, 900 ng, 1 mg, 10 mg, 100 mg, 200 mg, 300 mg, 400 mg, 500 mg, 600 mg, 700 mg, 800 mg, 900 mg, 1 g, 2 g, 3 g, 4 g, or 5 g.

In some embodiments, a non-viral approach of introducing a CAR and/or TCR sequence to a cell can include electroporation. Electroporation can be performed using, for example, the Neon® Transfection System (ThermoFisher Scientific) or the AMAXA® Nucleofector (AMAXA® Biosystems). Electroporation parameters may be adjusted to optimize transfection efficiency and/or cell viability. Electroporation devices can have multiple electrical wave form pulse settings such as exponential decay, time constant and square wave. Every cell type has a unique optimal Field Strength (E) that is dependent on the pulse parameters applied (e.g., voltage, capacitance and resistance). Application of optimal field strength causes electropermeabilization through induction of transmembrane voltage, which allows nucleic acids to pass through the cell membrane. In some cases, the electroporation pulse voltage, the electroporation pulse width, number of pulses, cell density, and tip type may be adjusted to optimize transfection efficiency and/or cell viability.

In some embodiments, electroporation pulse voltage may be varied to optimize transfection efficiency and/or cell viability. In some cases, the electroporation voltage may be less than about 500 volts. In some cases, the electroporation voltage may be at least about 500 volts, at least about 600 volts, at least about 700 volts, at least about 800 volts, at least about 900 volts, at least about 1000 volts, at least about 1100 volts, at least about 1200 volts, at least about 1300 volts, at least about 1400 volts, at least about 1500 volts, at least about 1600 volts, at least about 1700 volts, at least about 1800 volts, at least about 1900 volts, at least about 2000 volts, at least about 2100 volts, at least about 2200 volts, at least about 2300 volts, at least about 2400 volts, at least about 2500 volts, at least about 2600 volts, at least about 2700 volts, at least about 2800 volts, at least about 2900 volts, or at least about 3000 volts. In some cases, the electroporation pulse voltage required for optimal transfection efficiency and/or cell viability may be specific to the cell type. For example, an electroporation voltage of 1900 volts may optimal (e.g., provide the highest viability and/or transfection efficiency) for macrophage cells. In another example, an electroporation voltage of about 1350 volts may optimal (e.g., provide the highest viability and/or transfection efficiency) for Jurkat cells or primary human cells such as T cells. In some cases, a range of electroporation voltages may be optimal for a given cell type. For example, an electroporation voltage between about 1000 volts and about 1300 volts may optimal (e.g., provide the highest viability and/or transfection efficiency) for human 578T cells. In some cases, a primary cell can be a primary lymphocyte. In some cases, a population of primary cells can be a population of lymphocytes.

In some embodiments, electroporation pulse width may be varied to optimize transfection efficiency and/or cell viability. In some cases, the electroporation pulse width may be less than about 5 milliseconds. In some cases, the electroporation width may be at least about 5 milliseconds, at least about 6 milliseconds, at least about 7 milliseconds, at least about 8 milliseconds, at least about 9 milliseconds, at least about 10 milliseconds, at least about 11 milliseconds, at least about 12 milliseconds, at least about 13 milliseconds, at least about 14 milliseconds, at least about 15 milliseconds, at least about 16 milliseconds, at least about 17 milliseconds, at least about 18 milliseconds, at least about 19 milliseconds, at least about 20 milliseconds, at least about 21 milliseconds, at least about 22 milliseconds, at least about 23 milliseconds, at least about 24 milliseconds, at least about 25 milliseconds, at least about 26 milliseconds, at least about 27 milliseconds, at least about 28 milliseconds, at least about 29 milliseconds, at least about 30 milliseconds, at least about 31 milliseconds, at least about 32 milliseconds, at least about 33 milliseconds, at least about 34 milliseconds, at least about 35 milliseconds, at least about 36 milliseconds, at least about 37 milliseconds, at least about 38 milliseconds, at least about 39 milliseconds, at least about 40 milliseconds, at least about 41 milliseconds, at least about 42 milliseconds, at least about 43 milliseconds, at least about 44 milliseconds, at least about 45 milliseconds, at least about 46 milliseconds, at least about 47 milliseconds, at least about 48 milliseconds, at least about 49 milliseconds, or at least about 50 milliseconds. In some cases, the electroporation pulse width required for optimal transfection efficiency and/or cell viability may be specific to the cell type. For example, an electroporation pulse width of 30 milliseconds may optimal (e.g., provide the highest viability and/or transfection efficiency) for macrophage cells. In another example, an electroporation width of about 10 milliseconds may optimal (e.g., provide the highest viability and/or transfection efficiency) for Jurkat cells. In some cases, a range of electroporation widths may be optimal for a given cell type. For example, an electroporation width between about 20 milliseconds and about 30 milliseconds may optimal (e.g., provide the highest viability and/or transfection efficiency) for human 578T cells.

In some embodiments, the number of electroporation pulses may be varied to optimize transfection efficiency and/or cell viability. In some cases, electroporation may comprise a single pulse. In some cases, electroporation may comprise more than one pulse. In some cases, electroporation may comprise 2 pulses, 3 pulses, 4 pulses, 5 pulses 6 pulses, 7 pulses, 8 pulses, 9 pulses, or 10 or more pulses. In some cases, the number of electroporation pulses required for optimal transfection efficiency and/or cell viability may be specific to the cell type. For example, electroporation with a single pulse may be optimal (e.g., provide the highest viability and/or transfection efficiency) for macrophage cells. In another example, electroporation with a 3 pulses may be optimal (e.g., provide the highest viability and/or transfection efficiency) for primary cells. In some cases, a range of electroporation widths may be optimal for a given cell type. For example, electroporation with between about 1 to about 3 pulses may be optimal (e.g., provide the highest viability and/or transfection efficiency) for human cells.

In some cases, the starting cell density for electroporation may be varied to optimize transfection efficiency and/or cell viability. In some cases, the starting cell density for electroporation may be less than about 1×10⁵ cells. In some cases, the starting cell density for electroporation may be at least about 1×10⁵ cells, at least about 2×10⁵ cells, at least about 3×10⁵ cells, at least about 4×10⁵ cells, at least about 5×10⁵ cells, at least about 6×10⁵ cells, at least about 7×10⁵ cells, at least about 8×10⁵ cells, at least about 9×10⁵ cells, at least about 1×10⁶ cells, at least about 1.5×10⁶ cells, at least about 2×10⁶ cells, at least about 2.5×10⁶ cells, at least about 3×10⁶ cells, at least about 3.5×10⁶ cells, at least about 4×10⁶ cells, at least about 4.5×10⁶ cells, at least about 5×10⁶ cells, at least about 5.5×10⁶ cells, at least about 6×10⁶ cells, at least about 6.5×10⁶ cells, at least about 7×10⁶ cells, at least about 7.5×10⁶ cells, at least about 8×10⁶ cells, at least about 8.5×10⁶ cells, at least about 9×10⁶ cells, at least about 9.5×10⁶ cells, at least about 1×10⁷ cells, at least about 1.2×10⁷ cells, at least about 1.4×10⁷ cells, at least about 1.6×10⁷ cells, at least about 1.8×10⁷ cells, at least about 2×10⁷ cells, at least about 2.2×10⁷ cells, at least about 2.4×10⁷ cells, at least about 2.6×10⁷ cells, at least about 2.8×10⁷ cells, at least about 3×10⁷ cells, at least about 3.2×10⁷ cells, at least about 3.4×10⁷ cells, at least about 3.6×10⁷ cells, at least about 3.8×10⁷ cells, at least about 4×10⁷ cells, at least about 4.2×10⁷ cells, at least about 4.4×10⁷ cells, at least about 4.6×10⁷ cells, at least about 4.8×10⁷ cells, or at least about 5×10⁷ cells. In some cases, the starting cell density for electroporation required for optimal transfection efficiency and/or cell viability may be specific to the cell type. For example, a starting cell density for electroporation of 1.5×10⁶ cells may optimal (e.g., provide the highest viability and/or transfection efficiency) for macrophage cells. In another example, a starting cell density for electroporation of 5×10⁶ cells may optimal (e.g., provide the highest viability and/or transfection efficiency) for human cells. In some cases, a range of starting cell densities for electroporation may be optimal for a given cell type. For example, a starting cell density for electroporation between of 5.6×10⁶ and 5×10⁷ cells may optimal (e.g., provide the highest viability and/or transfection efficiency) for human cells such as T cells.

A method for treating a lymphoid malignancy is provided. The method can comprise administering to a patient in need thereof a population of engineered immune cells. An individual engineered immune cell of the population can comprise one or more chimeric antigen receptors (CARs) comprising a binding moiety, where the binding moiety can comprise an antigen binding domain capable of binding to an immune cell antigen or CD19, and where each CAR of the one or more CARs can further comprise a transmembrane domain and an intracellular signaling domain. An individual engineered immune cell of the population can further comprise an enhancer moiety capable of enhancing one or more activities of the engineered immune cell. An endogenous T cell receptor (TCR) of the engineered immune cell may be inactivated. In some cases, the number of affected cells in peripheral blood or the number of affected cells in bone marrow of the patient can be reduced by at least about 10%, 15%, 20%, 25%, 30%, 35%, 40%, 45%, 50%, 55%, 60%, 65%, 70%, 75%, 80%, 85%, 90%, 95% or more within a period (e.g., 3 weeks) after a last dosing of the engineered immune cells. In some cases, the period after a last dosing of the engineered immune cell can be about 1 week, 2 weeks, 3 weeks, 4 weeks, 5 weeks or more. The number of any one or more of autologous T cell, granulocyte, and NK cell in peripheral blood of the patient can start to increase within a period (e.g., 3 weeks) after a last dosing of the engineered immune cells. In some cases, the period after a last dosing of the engineered immune cell can be about 1 week, 2 weeks, 3 weeks, 4 weeks, 5 weeks or more.

The enhancer moiety can enhance one or more activities of the engineered immune cell. The enhancer moiety can be configured to constitutively enhance the one or more activities of the engineered immune cell. The enhancer moiety can be configured to constitutively upregulate one or more intracellular signaling pathways of the engineered immune cell. The one or more intracellular signaling pathways can be one or more cytokine signaling pathways. The enhancer moiety can be a cytokine or a cytokine receptor. The enhancer moiety can be selected from the group consisting of IL-2, IL-3, IL-4, IL-6, IL-7, IL-8, IL-10, IL-11, IL-12, IL-15, IL-17, IL-18, IL-21, IL-23, PD-1, PD-L1, CD122, CSF1R, CTAL-4, TIM-3, CCL21, CCL19, TGFR beta, receptors for the same, functional fragments thereof, functional variants thereof, and combinations thereof.

The engineered immune cell can further comprise an inducible cell death moiety capable of effecting death of the cell upon contacting the inducible cell death moiety with a cell death activator. The inducible cell death moiety can be selected from the group consisting of rapaCasp9, iCasp9, HSV-TK, CD20, ΔCD20, mTMPK, ΔCD19, RQR8, Her2t, CD30, BCMA, and EGFRt. For example, the inducible cell death moiety can be EGFRt, and the cell death activator can be an antibody or an antigen binding fragment thereof that binds EGFRt. For another example, the inducible cell death moiety can be HSV-TK, and the cell death activator can be GCV. For another example, the inducible cell death moiety can be iCasp9, and the cell death activator can be AP1903.

A gene encoding an endogenous surface marker of the cell can be inactivated. The endogenous surface marker can be capable of binding to the first antigen binding domain when expressed. The endogenous surface marker can be CD2, CD3, CD5, or CD7.

The number of any one or more of autologous T cell, granulocyte, and NK cell in peripheral blood of the patient may start to increase before the number of affected cells in peripheral blood or the number of affected cells in bone marrow is reduced by at least about 10%, 15%, 20%, 25%, 30%, 35%, 40%, 45%, 50%, 55%, 60%, 65%, 70%, 75%, 80%, 85%, 90%, 95% or more. The number of any one or more of autologous T cell, granulocyte, and NK cell in peripheral blood may start to increase after the number of affected cells in peripheral blood or the number of affected cells in bone marrow is reduced by at least about 10⁰/a, 15%, 20%, 25%, 30%, 35%, 40%, 45%, 50%, 55%, 60%, 65%, 70%, 75%, 80%, 85%, 90%, 95% or more.

### EXAMPLES

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Example 1 Study of U-CAR T Cells Expressing CD7/CD19 Dual CAR

### General Materials and Methods

Isolation of Peripheral Blood Mononuclear Cells (PBMCs) from Donor Blood and Expansion of T Cells

Peripheral blood mononuclear cells (PBMCs) were isolated from donor blood by using Histopaque-1077 (Sigma-Aldrich) through density gradient centrifuge. Then T cells were enriched, activated by magnetic beads coupled with anti-CD3/anti-CD28, cultured and expanded.

### Cell Lines and Culture of PBMCs

Raji cells (Burkitt's lymphoma cells, ATCC-CCL86);

Raji-filuc cell line (obtained by screening Raji cells transfected with lentivirus having firefly luciferase);

293T cells (ATCC-CRL3216);

CCRF-CEM cells (ATCC-CCL119);

PBNK: peripheral blood NK sorted from PBMC by using CD56 microbeads and cultured in NK serum free medium kit II (Cyagen Biosciences)+10% FBS+900 IU/ml IL2 (PeproTech);

NK92 cells (ATCC-CRL2407);

NK92-ffluc cells (obtained by screening NK92 cells transfected with lentivirus having firefly luciferase);

Raji cells, Raji-filuc cell line, and K562 cells were cultured in RPMI1640 medium, and

293T cells were cultured in DMEM medium. Both RPMI1640 and DMEM were supplemented with 10% (v/v) fetal bovine serum and 100 U/ml penicillin and streptomycin, 2 mM glutamine and 1 mM sodium pyruvate. All of the cells were cultured in an incubator at 37° C., 5% CO₂.

NK92 cells were cultured in RPMI1640 medium supplemented with 10% (v/v) fetal bovine serum, 2 mM glutamine, 1 mM sodium pyruvate, 1% NEAA, 0.1 mM mercaptoethanol and 200 IU/ml rhIL2.

T cells and the obtained CAR-T cells were cultured in X-vivo15 medium (containing 5% FBS, 2 mM L-glutamine, 1 mM sodium pyruvate and 300 IU/ml rhIL2). The culture medium for CAR-T cells was further supplemented with rhIL-2 (ThermoFisher Scientific) at a final concentration of 300 IU/ml every two days. All of the cells were cultured in an incubator at 37° C., 5% CO2.

### 1.1 Design of CD7/CD19 Dual CARs in Loon and Parallel Structures.

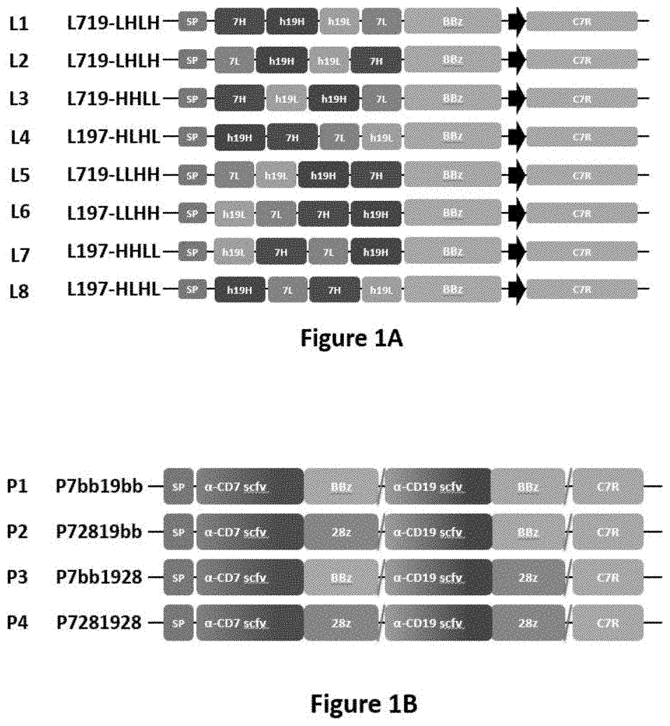

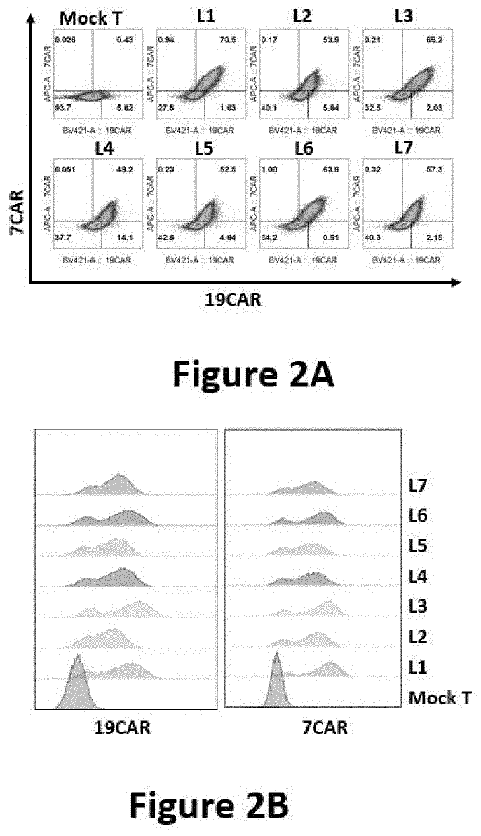

The design of the CD7/CD19 dual CARs targeting CD19 and CD7 are in Loop (FIG. 1A) and Parallel (FIG. 1B) structures as illustrated below:

The Loop structures include: L719-LHLH, L719-LLHH, L719-HHLL, L719-HLHL, L197-LHLH, L197-LLHH, L197-HHLL, L197-HLHL, and the sequences of the Loop structures are as set forth in SEQ ID NO. 53 to 60.

CD7/CD19 Loop dual CAR connected with a constitutively activated IL7 receptor C7R: L719-HHLL-C7R, amino acid sequence as set forth in SEQ ID NO 61.

The Parallel structures include: P7bb19bb, P72819bb, P7bb1928, P7281928 (by using a linker with P2A), and the sequences of the Parallel structures are as set forth in SEQ ID NO 62-65.

CD7/CD19 Parallel dual CAR connected with a constitutively activated IL7 receptor C7R: P7bb19bb, amino acid sequence as set forth in SEQ ID NO 66.

### 1.2 Test for the CD7/CD19 CAR-T Cells in Loop Structure

### Expression of the Dual CARs and the Knockout Efficiency

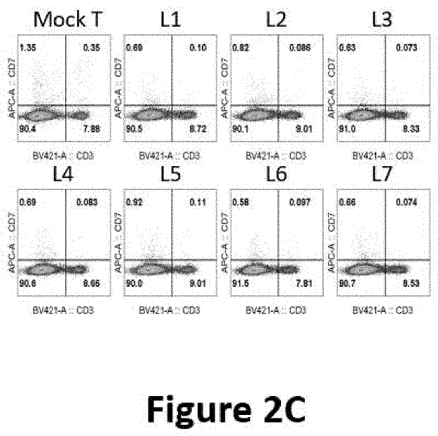

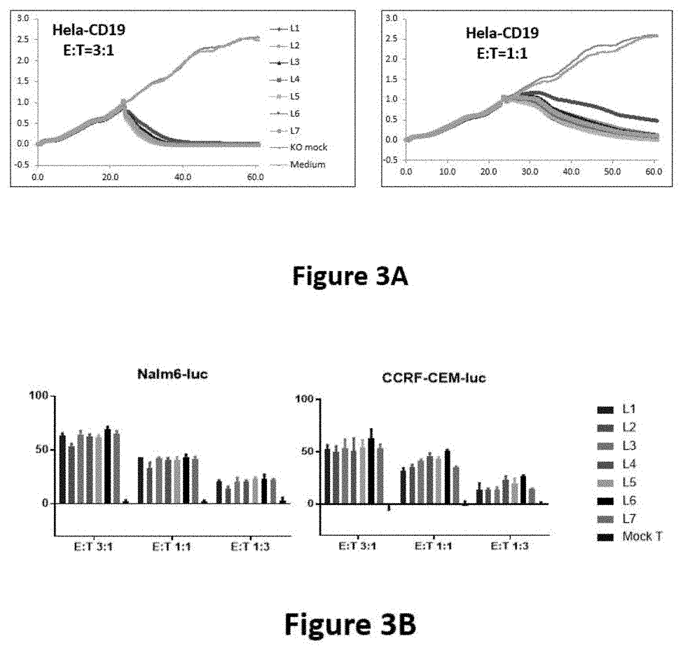

We compared 7 Loop CARs (L719-LHLH, L719-LLHH, L719-HHLL, L719-HLHL, L197-LHLH, L197-LLHH, L197-HHLL, referred as L1-L7, respectively), and Mock T was used as a negative control. As shown in FIGS. 2A and 2B, the expression of 7 Loop dual CARs was diagonally distributed, indicating successful expression of the dual CARs, and among which, L1, L3, L7 showed better expression. FIG. 3C shows the knockout efficiencies of CD7 and CD3 (TRAC), and it can be seen that in Mock T and CD7/CD19 CAR-T cells, the knockout efficiencies were 90% or above.

### In Vitro Killing Effects of Dual CARs on CD19+ Cells and CD7+ Cells

First, we compared the killing effects of different CARs on CD 19+ target cells. xCELLigence Real-Time Cell Analyzer (RTCA) experiment showed that 7 Loop CARs had potent killing effects on HeLa-CD19+ cells with 3:1 and 1:1 of E:T ratio (FIG. 3A). Furthermore, we compared the killing effects of different CARs on CD7+(CCRF) and CD19+(NALM6) target cells. All the Loop CD7/CD19 CAR-T cells showed potent killing activity on both CCRF and NALM6 cells in Luciferase killing assay (FIG. 3B).

### 1.3 Test for the CD7/CD19CAR-T Cells in Parallel Structure

4 Parallel CD7/CD19 dual CAR structures were prepared and compared with the Loop CD7/CD19 dual CAR (L197-HHLL, also named as L197) and CD7 single CAR.

### 1) Expression of Parallel CD7/CD19 Dual CARs.

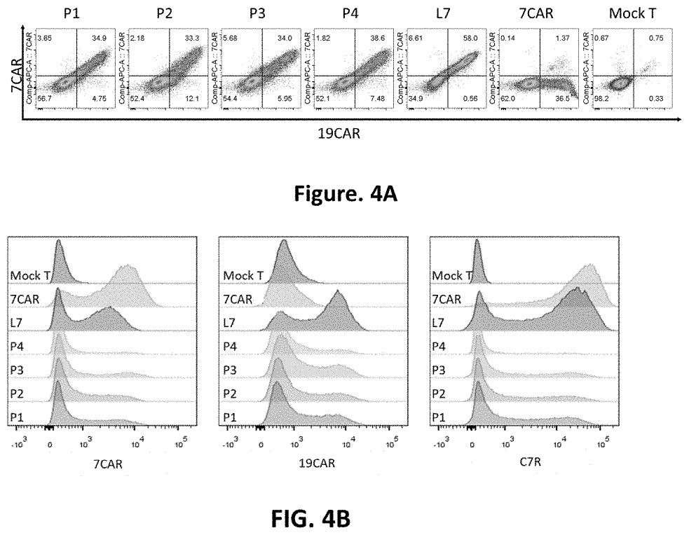

FIG. 4A and FIG. 4B show that the parallel CD7/CD19 dual CARs as well as the enhancer C7R were successfully expressed.

### 2) Killing Potency of Parallel CD7/CD19 Dual CARs.

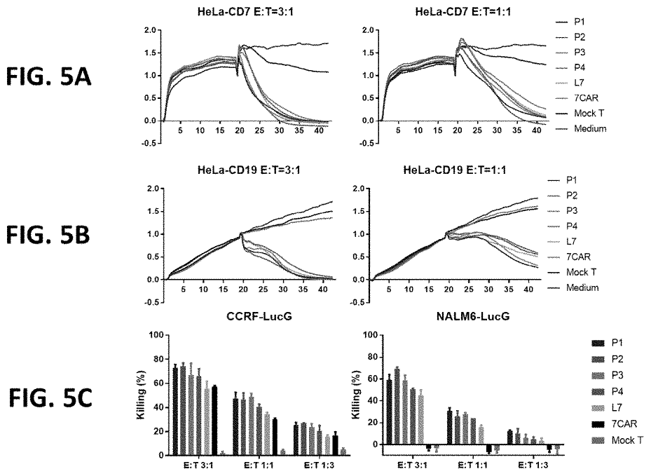

Real-time Cell Analysis (RTCA) (FIG. 5A and FIG. 5B) shows that Parallel CD7/CD19 dual CARs had better killing effects on HeLa-CD7+ (FIG. 5A) and HeLa-CD19+ (FIG. 5B) cells than the Loop CD7/CD19 dual CAR. FIG. 5C shows that, Parallel CD7/CD19 dual CARs had better killing effects on CD7+ (CCRF) and CD19+ (NALM6) target cells as compare to the Loop CD7/CD19 dual CAR and CD7 single CAR in luciferase killing assay.

### 3) Release of the Cytokines of the CD7/CD19 Dual CARs.

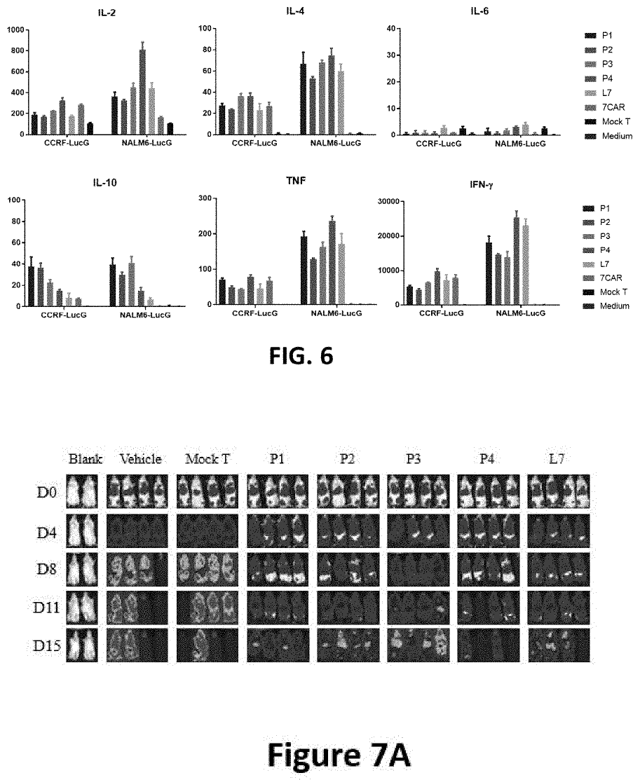

The CD7/CD19 dual CAR-T cells and CD19 positive tumor cells (NALM6) or CD7 positive tumor cells (CCRF), 100 ul each, were mixed in RPMI medium at 1:1 ratio to a density of 1×10⁶/ml for each cell, and then cultured overnight in a 96-well plate. The medium was then collected and subject to centrifuge and the released cytokine IFN-7, IL-2, IL-4, IL-6, IL-10 and TNF were detected by Cytokine bead array kit (CBA kit, BD Biosciences), as shown in FIG. 6.

### 4) In Vivo Clearance of CD7+ CCRF Tumor by Dual CARs.

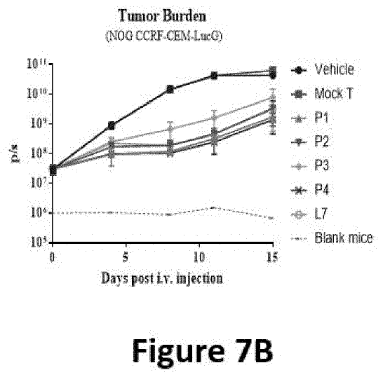

FIG. 7A and FIG. 7B show that the Parallel CD7/CD19 dual CARs and the Loop CD7/CD19 dual CAR all delayed the progression of CCRF-CEM tumors in vivo (CCRF-CEM intravenous model in NOG mice) as compared to the vehicle control.

While preferred embodiments of the present disclosure have been shown and described herein, such embodiments are provided by way of example only. Various alternatives to the embodiments described herein are optionally employed in practicing the disclosure. It is intended that the following claims define the scope of the disclosure and that methods and structures within the scope of these claims and their equivalents be covered thereby.

Sequence Listing

| SEQ ID | |
|---|---|
| NO. | Sequences |
| 1 | QVQLQESGPGLVKPSQTLSLTCTVSGVSLPDYGVSWIRQPPGKGLEWIGVIWGSETTYYNSALKS |
| | RVTISVDTSKNQFSLKLSSVTAADTAVYYCARHYYYGGSYAMDYWGQGTTVTVSS |
| 2 | QVQLQESGPGLVKPSQTLSLTCTVSGVSLPDYGVSWIRQPPGKGLEWIGVIWGSETTYYNSALKS |
| | RVTISKDTSKNQVSLKLSSVTAADTAVYYCAKHYYYGGSYAMDYWGQGTTVTVSS |
| 3 | QVQLQESGPGLVKPSQTLSLTCTVSGVSLPDYGVSWIRQPPGKGLEWIGVIWGSETTYYNSALKS |
| | RLTISKDTSKNQVSLKLSSVTAADTAVYYCAKHYYYGGSYAMDYWGQGTTVTVSS |
| 4 | QVQLQESGPGLVKPSQTLSLTCTVSGVSLPDYGVSWIRQPPGKGLEWLGVIWGSETTYYNSALKS |
| | RLTISKDTSKNQVSLKLSSVTAADTAVYYCAKHYYYGGSYAMDYWGQGTTVTVSS |
| 5 | QVQLQESGPGLVKPSQTLSVTCTVSGVSLPDYGVSWIRQPPGKGLEWLGVIWGSETTYYNSALKS |
| | RLTISKDTSKNQVSLKLSSLTAADTAVYYCAKHYYYGGSYAMDYWGQGTTVTVSS |
| 6 | QVQLQESGPGLVKPSQTLSVTCTVSGVSLPDYGVSWIRQPPGKGLEWLGVIWGSETTYYNAALKS |
| | RLTISKDTSKNQVSLKLSSLTAADTAVYYCAKHYYYGGSYAMDYWGQGTTVTVSS |
| 7 | QVKLQESGPGLVKPSETLSLTCTVSGVSLPDYGVSWIRQPPGKGLEWLGVIWGSETTYYNSALKS |
| | RLTISKDTSKNQFSLKLSSVTAADTAVYYCAKHYYYGGSYAMDYWGQGTLVTVSS |
| 8 | QVKLQESGPGLVKPSETLSLTCTVSGVSLPDYGVSWIRQPPGKGLEWIGVIWGSETTYYNSALKS |
| | RVTISKDTSKSQVFLKLSSVTAADTAVYYCAKHYYYGGSYAMDYWGQGTLVTVSS |
| 9 | QVQLQESGPGLVKPSETLSLTCTVSGVSLPDYGVSWIRQPPGKGLEWLGVIWGSETTYYNSALKS |
| | RLTISKDTSKSQVSLKLSSVTAADTAVYYCAKHYYYGGSYAMDYWGQGTLVTVSS |
| 10 | QVKLQESGPGLVKPSETLSLTCTVSGVSLPDYGVSWIRQPPGKGLEWLGVIWGSETTYYNSALKS |
| | RLTISKDTSKSQVFLKLSSVTAADTAVYYCAKHYYYGGSYAMDYWGQGTLVTVSS |
| 11 | DIQMTQSPSSLSASVGDRVTITCRASQDISKYLNWYQQKPGKAPKLLIYHTSRLHSGVPSRFSGS |
| | GSGTDFTFTISSLQPEDIATYYCQQGNTLPYTFGGGTKLEIK |
| 12 | DIQMTQSPSSLSASVGDRVTITCRASQDISKYLNWYQQKPGKAPKLLIYHTSRLHSGVPSRFSGS |
| | GSGTDYTFTISSLQPEDIATYYCQQGNTLPYTFGGGTKLEIK |
| 13 | DIQMTQSPSSLSASVGDRVTITCRASQDISKYLNWYQQKPGKAPKLLIYHTSRLHSGVPSRFSGS |
| | GSGTDYTLTISSLQPEDIATYYCQQGNTLPYTFGGGTKLEIK |
| 14 | DIQMTQSPSSLSASVGDRVTITCRASQDISKYLNWYQQKPGKAPKLLIYHTSRLHSGVPSRFSGS |
| | GSGTDYTLTISSLQPEDIATYYCQQANTLPYTFGGGTKLEIK |
| 15 | DIQMTQSPSSLSASVGDRVTISCRASQDISKYLNWYQQKPGKAPKLLIYHTSRLHSGVPSRFSGS |
| | GSGTDYTFTISSLQPEDIATYFCQQGNTLPYTFGGGTKLEIK |
| 16 | DIQMTQSPSSLSASVGDRVTITCRASQDISKYLNWYQQKPGGAVKLLIYHTSRLHSGVPSRFSGS |
| | GSGTDYTFTISSLQPEDIATYFCQQGNTLPYTFGGGTKLEIK |
| 17 | DIQMTQSPSSLSASVGDRVTISCRASQDISKYLNWYQQKPDGTVKLLIYHTSRLHSGVPSRFSGS |
| | GSGTDFTFTISSLQPEDIATYFCQQGNTLPYTFGGGTKLEIK |
| 18 | QVKLQESGPGLVKPSETLSLTCTVS |
| 19 | WIRQPPGKGLEWLG |
| 20 | RLTISKDTSKNQFSLKLSSVTAADTAVYYCAK |
| 21 | WGQGTLVTVSS |
| 22 | DIQMTQSPSSLSASVGDRVTITC |
| 23 | WYQQKPGGAVKLLIY |
| 24 | GVPSRFSGSGSGTDYTFTISSLQPEDIATYFC |
| 25 | FGGGTKLEIK |
| 26 | GVSLPDYGVS |
| 27 | VIWGSETTYYNSALKS |
| 28 | VIWGSETTYYNAALKS |
| 29 | HYYYGGSYAMDY |
| 30 | RASQDISKYLN |
| 31 | HTSRLHS |
| 32 | QQGNTLPYT |
| 33 | QQANTLPYT |
| 34 | QVQLQESGPGLVKPSQTLSLTCTVS |
| 35 | QVQLQESGPGLVKPSQTLSVTCTVS |
| 36 | QVQLQESGPGLVKPSETLSLTCTVS |
| 37 | WIRQPPGKGLEWIG |
| 38 | RVTISVDTSKNQFSLKLSSVTAADTAVYYCAR |
| 39 | RVTISKDTSKNQVSLKLSSVTAADTAVYYCAK |
| 40 | RLTISKDTSKNQVSLKLSSVTAADTAVYYCAK |
| 41 | RLTISKDTSKNQVSLKLSSLTAADTAVYYCAK |
| 42 | RVTISKDTSKSQVFLKLSSVTAADTAVYYCAK |
| 43 | RLTISKDTSKSQVSLKLSSVTAADTAVYYCAK |
| 44 | RLTISKDTSKSQVFLKLSSVTAADTAVYYCAK |
| 45 | WGQGTTVTVSS |
| 46 | DIQMTQSPSSLSASVGDRVTISC |
| 47 | WYQQKPGKAPKLLIY |
| 48 | WYQQKPDGTVKLLIY |
| 49 | GVPSRFSGSGSGTDFTFTISSLQPEDIATYYC |
| 50 | GVPSRFSGSGSGTDYTFTISSLQPEDIATYYC |
| 51 | GVPSRFSGSGSGTDYTLTISSLQPEDIATYYC |
| 52 | GVPSRFSGSGSGTDFTFTISSLQPEDIATYFC |
| 53 | MALPVTALLLPLALLLHAARPQVKLQESGGGLVKPGGSLKLSCAASGFTFSSYAMSWVRQTPEKR |
| | LEWVATISSGGSYTYYPDSVKGRFTISRDNAKNTLYLQMSSLRSEDTAMYYCARQDGYYPGWFAN |
| | WGQGTTVTVSSGGGGSQVQLQESGPGLVKPSETLSLTCTVSGVSLPDYGVSWIRQPPGKGLEWLG |
| | VIWGSETTYYNSALKSRLTISKDTSKSQVSLKLSSVTAADTAVYYCAKHYYYGGSYAMDYWGQGT |
| | LVTVSSGSTSGSGKPGSGEGSTKGDIQMTQSPSSLSASVGDRVTISCRASQDISKYLNWYQQKPG |
| | KAPKLLIYHTSRLHSGVPSRFSGSGSGTDYTFTISSLQPEDIATYFCQQGNTLPYTFGGGTKLEI |
| | KGGGGSDIELTQSPAIMSASLGEEITLTCSASSSVSYMHWYQQKSGTSPKLLIYSTSNLASGVPS |
| | RFSGSGSGTFYSLTISSVEAEDAADYYCHQWSSYTFGGGTKLEIKRTTTPAPRPPTPAPTIASQP |
| | LSLRPEACRPAAGGAVHTRGLDFACDIYIWAPLAGTCGVLLLSLVITLYCKRGRKKLLYIFKQPF |
| | MRPVQTTQEEDGCSCRFPEEEEGGCELRVKFSRSADAPAYKQGQNQLYNELNLGRREEYDVLDKR |
| | RGRDPEMGGKPRRKNPQEGLYNELQKDKMAEAYSEIGMKGERRRGKGHDGLYQGLSTATKDTYDA |
| | LHMQALPPR* |
| 54 | MALPVTALLLPLALLLHAARPDIELTQSPAIMSASLGEEITLTCSASSSVSYMHWYQQKSGTSPK |
| | LLIYSTSNLASGVPSRFSGSGSGTFYSLTISSVEAEDAADYYCHQWSSYTFGGGTKLEIKRGGGG |
| | SQVQLQESGPGLVKPSETLSLTCTVSGVSLPDYGVSWIRQPPGKGLEWLGVIWGSETTYYNSALK |
| | SRLTISKDTSKSQVSLKLSSVTAADTAVYYCAKHYYYGGSYAMDYWGQGTLVTVSSGSTSGSGKP |
| | GSGEGSTKGDIQMTQSPSSLSASVGDRVTISCRASQDISKYLNWYQQKPGKAPKLLIYHTSRLHS |
| | GVPSRFSGSGSGTDYTFTISSLQPEDIATYFCQQGNTLPYTFGGGTKLEIKGGGGSQVKLQESGG |
| | GLVKPGGSLKLSCAASGFTFSSYAMSWVRQTPEKRLEWVATISSGGSYTYYPDSVKGRFTISRDN |
| | AKNTLYLQMSSLRSEDTAMYYCARQDGYYPGWFANWGQGTTVTVSSTTTPAPRPPTPAPTIASQP |
| | LSLRPEACRPAAGGAVHTRGLDFACDIYIWAPLAGTCGVLLLSLVITLYCKRGRKKLLYIFKQPF |
| | MRPVQTTQEEDGCSCRFPEEEEGGCELRVKFSRSADAPAYKQGQNQLYNELNLGRREEYDVLDKR |
| | RGRDPEMGGKPRRKNPQEGLYNELQKDKMAEAYSEIGMKGERRRGKGHDGLYQGLSTATKDTYDA |
| | LHMQALPPR* |
| 55 | MALPVTALLLPLALLLHAARPQVKLQESGGGLVKPGGSLKLSCAASGFTFSSYAMSWVRQTPEKR |
| | LEWVATISSGGSYTYYPDSVKGRFTISRDNAKNTLYLQMSSLRSEDTAMYYCARQDGYYPGWFAN |
| | WGQGTTVTVSSGGGGSDIQMTQSPSSLSASVGDRVTISCRASQDISKYLNWYQQKPGKAPKLLIY |
| | HTSRLHSGVPSRFSGSGSGTDYTFTISSLQPEDIATYFCQQGNTLPYTFGGGTKLEIKGSTSGSG |
| | KPGSGEGSTKGQVQLQESGPGLVKPSETLSLTCTVSGVSLPDYGVSWIRQPPGKGLEWLGVIWGS |
| | ETTYYNSALKSRLTISKDTSKSQVSLKLSSVTAADTAVYYCAKHYYYGGSYAMDYWGQGTLVTVS |
| | SGGGGSDIELTQSPAIMSASLGEEITLTCSASSSVSYMHWYQQKSGTSPKLLIYSTSNLASGVPS |
| | RFSGSGSGTFYSLTISSVEAEDAADYYCHQWSSYTFGGGTKLEIKRTTTPAPRPPTPAPTIASQP |
| | LSLRPEACRPAAGGAVHTRGLDFACDIYIWAPLAGTCGVLLLSLVITLYCKRGRKKLLYIFKQPF |
| | MRPVQTTQEEDGCSCRFPEEEEGGCELRVKFSRSADAPAYKQGQNQLYNELNLGRREEYDVLDKR |
| | RGRDPEMGGKPRRKNPQEGLYNELQKDKMAEAYSEIGMKGERRRGKGHDGLYQGLSTATKDTYDA |
| | LHMQALPPR* |
| 56 | MALPVTALLLPLALLLHAARPDIELTQSPAIMSASLGEEITLTCSASSSVSYMHWYQQKSGTSPK |
| | LLIYSTSNLASGVPSRFSGSGSGTFYSLTISSVEAEDAADYYCHQWSSYTFGGGTKLEIKRGGGG |
| | SDIQMTQSPSSLSASVGDRVTISCRASQDISKYLNWYQQKPGKAPKLLIYHTSRLHSGVPSRFSG |
| | SGSGTDYTFTISSLQPEDIATYFCQQGNTLPYTFGGGTKLEIKGSTSGSGKPGSGEGSTKGQVQL |
| | QESGPGLVKPSETLSLTCTVSGVSLPDYGVSWIRQPPGKGLEWLGVIWGSETTYYNSALKSRLTI |
| | SKDTSKSQVSLKLSSVTAADTAVYYCAKHYYYGGSYAMDYWGQGTLVTVSSGGGGSQVKLQESGG |
| | GLVKPGGSLKLSCAASGFTFSSYAMSWVRQTPEKRLEWVATISSGGSYTYYPDSVKGRFTISRDN |
| | AKNTLYLQMSSLRSEDTAMYYCARQDGYYPGWFANWGQGTTVTVSSTTTPAPRPPTPAPTIASQP |
| | LSLRPEACRPAAGGAVHTRGLDFACDIYIWAPLAGTCGVLLLSLVITLYCKRGRKKLLYIFKQPF |
| | MRPVQTTQEEDGCSCRFPEEEEGGCELRVKFSRSADAPAYKQGQNQLYNELNLGRREEYDVLDKR |
| | RGRDPEMGGKPRRKNPQEGLYNELQKDKMAEAYSEIGMKGERRRGKGHDGLYQGLSTATKDTYDA |
| | LHMQALPPR* |
| 57 | MALPVTALLLPLALLLHAARPQVQLQESGPGLVKPSETLSLTCTVSGVSLPDYGVSWIRQPPGKG |
| | LEWLGVIWGSETTYYNSALKSRLTISKDTSKSQVSLKLSSVTAADTAVYYCAKHYYYGGSYAMDY |
| | WGQGTLVTVSSGGGGSQVKLQESGGGLVKPGGSLKLSCAASGFTFSSYAMSWVRQTPEKRLEWVA |
| | TISSGGSYTYYPDSVKGRFTISRDNAKNTLYLQMSSLRSEDTAMYYCARQDGYYPGWFANWGQGT |
| | TVTVSSGSTSGSGKPGSGEGSTKGDIELTQSPAIMSASLGEEITLTCSASSSVSYMHWYQQKSGT |
| | SPKLLIYSTSNLASGVPSRFSGSGSGTFYSLTISSVEAEDAADYYCHQWSSYTFGGGTKLEIKRG |
| | GGGSDIQMTQSPSSLSASVGDRVTISCRASQDISKYLNWYQQKPGKAPKLLIYHTSRLHSGVPSR |
| | FSGSGSGTDYTFTISSLQPEDIATYFCQQGNTLPYTFGGGTKLEIKTTTPAPRPPTPAPTIASQP |
| | LSLRPEACRPAAGGAVHTRGLDFACDIYIWAPLAGTCGVLLLSLVITLYCKRGRKKLLYIFKQPF |
| | MRPVQTTQEEDGCSCRFPEEEEGGCELRVKFSRSADAPAYKQGQNQLYNELNLGRREEYDVLDKR |
| | RGRDPEMGGKPRRKNPQEGLYNELQKDKMAEAYSEIGMKGERRRGKGHDGLYQGLSTATKDTYDA |
| | LHMQALPPR* |
| 58 | MALPVTALLLPLALLLHAARPDIQMTQSPSSLSASVGDRVTISCRASQDISKYLNWYQQKPGKAP |
| | KLLIYHTSRLHSGVPSRFSGSGSGTDYTFTISSLQPEDIATYFCQQGNTLPYTFGGGTKLEIKGG |
| | GGSDIELTQSPAIMSASLGEEITLTCSASSSVSYMHWYQQKSGTSPKLLIYSTSNLASGVPSRFS |
| | GSGSGTFYSLTISSVEAEDAADYYCHQWSSYTFGGGTKLEIKRGSTSGSGKPGSGEGSTKGQVKL |
| | QESGGGLVKPGGSLKLSCAASGFTFSSYAMSWVRQTPEKRLEWVATISSGGSYTYYPDSVKGRFT |
| | ISRDNAKNTLYLQMSSLRSEDTAMYYCARQDGYYPGWFANWGQGTTVTVSSGGGGSQVQLQESGP |
| | GLVKPSETLSLTCTVSGVSLPDYGVSWIRQPPGKGLEWLGVIWGSETTYYNSALKSRLTISKDTS |
| | KSQVSLKLSSVTAADTAVYYCAKHYYYGGSYAMDYWGQGTLVTVSSTTTPAPRPPTPAPTIASQP |
| | LSLRPEACRPAAGGAVHTRGLDFACDIYIWAPLAGTCGVLLLSLVITLYCKRGRKKLLYIFKQPF |
| | MRPVQTTQEEDGCSCRFPEEEEGGCELRVKFSRSADAPAYKQGQNQLYNELNLGRREEYDVLDKR |
| | RGRDPEMGGKPRRKNPQEGLYNELQKDKMAEAYSEIGMKGERRRGKGHDGLYQGLSTATKDTYDA |
| | LHMQALPPR* |
| 59 | MALPVTALLLPLALLLHAARPDIQMTQSPSSLSASVGDRVTISCRASQDISKYLNWYQQKPGKAP |
| | KLLIYHTSRLHSGVPSRFSGSGSGTDYTFTISSLQPEDIATYFCQQGNTLPYTFGGGTKLEIKGG |
| | GGSQVKLQESGGGLVKPGGSLKLSCAASGFTFSSYAMSWVRQTPEKRLEWVATISSGGSYTYYPD |
| | SVKGRFTISRDNAKNTLYLQMSSLRSEDTAMYYCARQDGYYPGWFANWGQGTTVTVSSGSTSGSG |
| | KPGSGEGSTKGDIELTQSPAIMSASLGEEITLTCSASSSVSYMHWYQQKSGTSPKLLIYSTSNLA |
| | SGVPSRFSGSGSGTFYSLTISSVEAEDAADYYCHQWSSYTFGGGTKLEIKRGGGGSQVQLQESGP |
| | GLVKPSETLSLTCTVSGVSLPDYGVSWIRQPPGKGLEWLGVIWGSETTYYNSALKSRLTISKDTS |
| | KSQVSLKLSSVTAADTAVYYCAKHYYYGGSYAMDYWGQGTLVTVSSTTTPAPRPPTPAPTIASQP |
| | LSLRPEACRPAAGGAVHTRGLDFACDIYIWAPLAGTCGVLLLSLVITLYCKRGRKKLLYIFKQPF |
| | MRPVQTTQEEDGCSCRFPEEEEGGCELRVKFSRSADAPAYKQGQNQLYNELNLGRREEYDVLDKR |
| | RGRDPEMGGKPRRKNPQEGLYNELQKDKMAEAYSEIGMKGERRRGKGHDGLYQGLSTATKDTYDA |
| | LHMQALPPR* |
| 60 | MALPVTALLLPLALLLHAARPQVQLQESGPGLVKPSETLSLTCTVSGVSLPDYGVSWIRQPPGKG |
| | LEWLGVIWGSETTYYNSALKSRLTISKDTSKSQVSLKLSSVTAADTAVYYCAKHYYYGGSYAMDY |
| | WGQGTLVTVSSGGGGSDIELTQSPAIMSASLGEEITLTCSASSSVSYMHWYQQKSGTSPKLLIYS |
| | TSNLASGVPSRFSGSGSGTFYSLTISSVEAEDAADYYCHQWSSYTFGGGTKLEIKRGSTSGSGKP |
| | GSGEGSTKGQVKLQESGGGLVKPGGSLKLSCAASGFTFSSYAMSWVRQTPEKRLEWVATISSGGS |
| | YTYYPDSVKGRFTISRDNAKNTLYLQMSSLRSEDTAMYYCARQDGYYPGWFANWGQGTTVTVSSG |
| | GGGSDIQMTQSPSSLSASVGDRVTISCRASQDISKYLNWYQQKPGKAPKLLIYHTSRLHSGVPSR |
| | FSGSGSGTDYTFTISSLQPEDIATYFCQQGNTLPYTFGGGTKLEIKTTTPAPRPPTPAPTIASQP |
| | LSLRPEACRPAAGGAVHTRGLDFACDIYIWAPLAGTCGVLLLSLVITLYCKRGRKKLLYIFKQPF |
| | MRPVQTTQEEDGCSCRFPEEEEGGCELRVKFSRSADAPAYKQGQNQLYNELNLGRREEYDVLDKR |
| | RGRDPEMGGKPRRKNPQEGLYNELQKDKMAEAYSEIGMKGERRRGKGHDGLYQGLSTATKDTYDA |
| | LHMQALPPR* |
| 61 | MALPVTALLLPLALLLHAARPQVKLQESGGGLVKPGGSLKLSCAASGFTFSSYAMSWVRQTPEKR |
| | LEWVATISSGGSYTYYPDSVKGRFTISRDNAKNTLYLQMSSLRSEDTAMYYCARQDGYYPGWFAN |
| | WGQGTTVTVSSGGGGSQVQLQESGPGLVKPSETLSLTCTVSGVSLPDYGVSWIRQPPGKGLEWLG |
| | VIWGSETTYYNSALKSRLTISKDTSKSQVSLKLSSVTAADTAVYYCAKHYYYGGSYAMDYWGQGT |
| | LVTVSSGSTSGSGKPGSGEGSTKGDIQMTQSPSSLSASVGDRVTISCRASQDISKYLNWYQQKPG |
| | KAPKLLIYHTSRLHSGVPSRFSGSGSGTDYTFTISSLQPEDIATYFCQQGNTLPYTFGGGTKLEI |
| | KGGGGSDIELTQSPAIMSASLGEEITLTCSASSSVSYMHWYQQKSGTSPKLLIYSTSNLASGVPS |
| | RFSGSGSGTFYSLTISSVEAEDAADYYCHQWSSYTFGGGTKLEIKRTTTPAPRPPTPAPTIASQP |
| | LSLRPEACRPAAGGAVHTRGLDFACDIYIWAPLAGTCGVLLLSLVITLYCKRGRKKLLYIFKQPF |
| | MRPVQTTQEEDGCSCRFPEEEEGGCELRVKFSRSADAPAYKQGQNQLYNELNLGRREEYDVLDKR |
| | RGRDPEMGGKPRRKNPQEGLYNELQKDKMAEAYSEIGMKGERRRGKGHDGLYQGLSTATKDTYDA |
| | LHMQALPPRGSGATNFSLLKQAGDVEENPGPMLVRRGARAGPRMPRGWTALCLLSLLPSGFMSLD |
| | NNGTATPELPTQGTFSNVSTNVSYQETTTPSTLGSTSLHPVSQHGNEATTNITETTVKFTSTSVI |
| | TSVYGNTNSSVQSQTSVISTVFTTPANVSTPETTLKPSLSPGNVSDLSTTSTSLATSPTKPYTSS |
| | SPILSDIKAEIKCSGIREVKLTQGICLEQNKTSSCAEFKKDRGEGLARVLCGEEQADADAGAQVC |
| | SLLLAQSEVRPQCLLLVLANRTEISSKLQLMKKHQSDLKKLGILDFTEQDVASHQSYSQKTPILL |
| | TCPTISILSFFSVALLVILACVLWKKRIKPIVWPSLPDHKKTLEHLCKKPRKNLNVSFNPESFLD |
| | CQIHRVDDIQARDEVEGFLQDTFPQQLEESEKQRLGGDVQSPNCPSEDVVITPESFGRDSSLTCL |
| | AGNVSACDAPILSSSRSLDCRESGKNGPHVYQDLLLSLGTTNSTLPPPFSLQSGILTLNPVAQGQ |
| | PILTSLGSNQEEAYVTMSSFYQNQ* |
| 62 | MALPVTALLLPLALLLHAARPDIELTQSPAIMSASLGEEITLTCSASSSVSYMHWYQQKSGTSPK |
| | LLIYSTSNLASGVPSRFSGSGSGTFYSLTISSVEAEDAADYYCHQWSSYTFGGGTKLEIKRGGGG |
| | SGGGGSGGGGSQVKLQESGGGLVKPGGSLKLSCAASGFTFSSYAMSWVRQTPEKRLEWVATISSG |
| | GSYTYYPDSVKGRFTISRDNAKNTLYLQMSSLRSEDTAMYYCARQDGYYPGWFANWGQGTTVTVS |
| | SSGTTTPAPRPPTPAPTIASQPLSLRPEACRPAAGGAVHTRGLDFACDIYIWAPLAGTCGVLLLS |
| | LVITLYCKRGRKKLLYIFKQPFMRPVQTTQEEDGCSCRFPEEEEGGCELRVKFSRSADAPAYKQG |
| | QNQLYNELNLGRREEYDVLDKRRGRDPEMGGKPRRKNPQEGLYNELQKDKMAEAYSEIGMKGERR |
| | RGKGHDGLYQGLSTATKDTYDALHMQALPPRGSGATNFSLLKQAGDVEENPGPMALPVTALLLPL |
| | ALLLHAARPDIQMTQSPSSLSASVGDRVTISCRASQDISKYLNWYQQKPGKAPKLLIYHTSRLHS |
| | GVPSRFSGSGSGTDYTFTISSLQPEDIATYFCQQGNTLPYTFGGGTKLEIKGGGGSGGGGSGGGG |
| | SQVQLQESGPGLVKPSETLSLTCTVSGVSLPDYGVSWIRQPPGKGLEWLGVIWGSETTYYNSALK |
| | SRLTISKDTSKSQVSLKLSSVTAADTAVYYCAKHYYYGGSYAMDYWGQGTLVTVSSTTTPAPRPP |
| | TPAPTIASQPLSLRPEACRPAAGGAVHTRGLDFACDIYIWAPLAGTCGVLLLSLVITLYCKRGRK |
| | KLLYIFKQPFMRPVQTTQEEDGCSCRFPEEEEGGCELRVKFSRSADAPAYKQGQNQLYNELNLGR |
| | REEYDVLDKRRGRDPEMGGKPRRKNPQEGLYNELQKDKMAEAYSEIGMKGERRRGKGHDGLYQGL |
| | STATKDTYDALHMQALPPR |
| 63 | MALPVTALLLPLALLLHAARPDIELTQSPAIMSASLGEEITLTCSASSSVSYMHWYQQKSGTSPK |
| | LLIYSTSNLASGVPSRFSGSGSGTFYSLTISSVEAEDAADYYCHQWSSYTFGGGTKLEIKRGGGG |
| | SGGGGSGGGGSQVKLQESGGGLVKPGGSLKLSCAASGFTFSSYAMSWVRQTPEKRLEWVATISSG |
| | GSYTYYPDSVKGRFTISRDNAKNTLYLQMSSLRSEDTAMYYCARQDGYYPGWFANWGQGTTVTVS |
| | SSGTTTPAPRPPTPAPTIASQPLSLRPEACRPAAGGAVHTRGLDFACDFWVLVVVGGVLACYSLL |
| | VTVAFIIFWVRSKRSRLLHSDYMNMTPRRPGPTRKHYQPYAPPRDFAAYRSRVKFSRSADAPAYK |
| | QGQNQLYNELNLGRREEYDVLDKRRGRDPEMGGKPRRKNPQEGLYNELQKDKMAEAYSEIGMKGE |
| | RRRGKGHDGLYQGLSTATKDTYDALHMQALPPRGSGATNFSLLKQAGDVEENPGPMALPVTALLL |
| | PLALLLHAARPDIQMTQSPSSLSASVGDRVTISCRASQDISKYLNWYQQKPGKAPKLLIYHTSRL |
| | HSGVPSRFSGSGSGTDYTFTISSLQPEDIATYFCQQGNTLPYTFGGGTKLEIKGGGGSGGGGSGG |
| | GGSQVQLQESGPGLVKPSETLSLTCTVSGVSLPDYGVSWIRQPPGKGLEWLGVIWGSETTYYNSA |
| | LKSRLTISKDTSKSQVSLKLSSVTAADTAVYYCAKHYYYGGSYAMDYWGQGTLVTVSSTTTPAPR |
| | PPTPAPTIASQPLSLRPEACRPAAGGAVHTRGLDFACDIYIWAPLAGTCGVLLLSLVITLYCKRG |
| | RKKLLYIFKQPFMRPVQTTQEEDGCSCRFPEEEEGGCELRVKFSRSADAPAYKQGQNQLYNELNL |
| | GRREEYDVLDKRRGRDPEMGGKPRRKNPQEGLYNELQKDKMAEAYSEIGMKGERRRGKGHDGLYQ |
| | GLSTATKDTYDALHMQALPPR |
| 64 | MALPVTALLLPLALLLHAARPDIELTQSPAIMSASLGEEITLTCSASSSVSYMHWYQQKSGTSPK |
| | LLIYSTSNLASGVPSRFSGSGSGTFYSLTISSVEAEDAADYYCHQWSSYTFGGGTKLEIKRGGGG |
| | SGGGGSGGGGSQVKLQESGGGLVKPGGSLKLSCAASGFTFSSYAMSWVRQTPEKRLEWVATISSG |
| | GSYTYYPDSVKGRFTISRDNAKNTLYLQMSSLRSEDTAMYYCARQDGYYPGWFANWGQGTTVTVS |
| | SSGTTTPAPRPPTPAPTIASQPLSLRPEACRPAAGGAVHTRGLDFACDIYIWAPLAGTCGVLLLS |
| | LVITLYCKRGRKKLLYIFKQPFMRPVQTTQEEDGCSCRFPEEEEGGCELRVKFSRSADAPAYKQG |
| | QNQLYNELNLGRREEYDVLDKRRGRDPEMGGKPRRKNPQEGLYNELQKDKMAEAYSEIGMKGERR |
| | RGKGHDGLYQGLSTATKDTYDALHMQALPPRGSGATNFSLLKQAGDVEENPGPMALPVTALLLPL |
| | ALLLHAARPDIQMTQSPSSLSASVGDRVTISCRASQDISKYLNWYQQKPGKAPKLLIYHTSRLHS |
| | GVPSRFSGSGSGTDYTFTISSLQPEDIATYFCQQGNTLPYTFGGGTKLEIKGGGGSGGGGSGGGG |
| | SQVQLQESGPGLVKPSETLSLTCTVSGVSLPDYGVSWIRQPPGKGLEWLGVIWGSETTYYNSALK |
| | SRLTISKDTSKSQVSLKLSSVTAADTAVYYCAKHYYYGGSYAMDYWGQGTLVTVSSTTTPAPRPP |
| | TPAPTIASQPLSLRPEACRPAAGGAVHTRGLDFACDFWVLVVVGGVLACYSLLVTVAFIIFWVRS |
| | KRSRLLHSDYMNMTPRRPGPTRKHYQPYAPPRDFAAYRSRVKFSRSADAPAYKQGQNQLYNELNL |
| | GRREEYDVLDKRRGRDPEMGGKPRRKNPQEGLYNELQKDKMAEAYSEIGMKGERRRGKGHDGLYQ |
| | GLSTATKDTYDALHMQALPPR |
| 65 | MALPVTALLLPLALLLHAARPDIELTQSPAIMSASLGEEITLTCSASSSVSYMHWYQQKSGTSPK |
| | LLIYSTSNLASGVPSRFSGSGSGTFYSLTISSVEAEDAADYYCHQWSSYTFGGGTKLEIKRGGGG |
| | SGGGGSGGGGSQVKLQESGGGLVKPGGSLKLSCAASGFTFSSYAMSWVRQTPEKRLEWVATISSG |
| | GSYTYYPDSVKGRFTISRDNAKNTLYLQMSSLRSEDTAMYYCARQDGYYPGWFANWGQGTTVTVS |
| | SSGTTTPAPRPPTPAPTIASQPLSLRPEACRPAAGGAVHTRGLDFACDFWVLVVVGGVLACYSLL |
| | VTVAFIIFWVRSKRSRLLHSDYMNMTPRRPGPTRKHYQPYAPPRDFAAYRSRVKFSRSADAPAYK |
| | QGQNQLYNELNLGRREEYDVLDKRRGRDPEMGGKPRRKNPQEGLYNELQKDKMAEAYSEIGMKGE |
| | RRRGKGHDGLYQGLSTATKDTYDALHMQALPPRGSGATNFSLLKQAGDVEENPGPMALPVTALLL |
| | PLALLLHAARPDIQMTQSPSSLSASVGDRVTISCRASQDISKYLNWYQQKPGKAPKLLIYHTSRL |
| | HSGVPSRFSGSGSGTDYTFTISSLQPEDIATYFCQQGNTLPYTFGGGTKLEIKGGGGSGGGGSGG |
| | GGSQVQLQESGPGLVKPSETLSLTCTVSGVSLPDYGVSWIRQPPGKGLEWLGVIWGSETTYYNSA |
| | LKSRLTISKDTSKSQVSLKLSSVTAADTAVYYCAKHYYYGGSYAMDYWGQGTLVTVSSTTTPAPR |
| | PPTPAPTIASQPLSLRPEACRPAAGGAVHTRGLDFACDFWVLVVVGGVLACYSLLVTVAFIIFWV |
| | RSKRSRLLHSDYMNMTPRRPGPTRKHYQPYAPPRDFAAYRSRVKFSRSADAPAYKQGQNQLYNEL |
| | NLGRREEYDVLDKRRGRDPEMGGKPRRKNPQEGLYNELQKDKMAEAYSEIGMKGERRRGKGHDGL |
| | YQGLSTATKDTYDALHMQALPPR |
| 66 | MALPVTALLLPLALLLHAARPDIELTQSPAIMSASLGEEITLTCSASSSVSYMHWYQQKSGTSPK |
| | LLIYSTSNLASGVPSRFSGSGSGTFYSLTISSVEAEDAADYYCHQWSSYTFGGGTKLEIKRGGGG |
| | SGGGGSGGGGSQVKLQESGGGLVKPGGSLKLSCAASGFTFSSYAMSWVRQTPEKRLEWVATISSG |
| | GSYTYYPDSVKGRFTISRDNAKNTLYLQMSSLRSEDTAMYYCARQDGYYPGWFANWGQGTTVTVS |
| | SSGTTTPAPRPPTPAPTIASQPLSLRPEACRPAAGGAVHTRGLDFACDIYIWAPLAGTCGVLLLS |
| | LVITLYCKRGRKKLLYIFKQPFMRPVQTTQEEDGCSCRFPEEEEGGCELRVKFSRSADAPAYKQG |
| | QNQLYNELNLGRREEYDVLDKRRGRDPEMGGKPRRKNPQEGLYNELQKDKMAEAYSEIGMKGERR |
| | RGKGHDGLYQGLSTATKDTYDALHMQALPPRGSGATNFSLLKQAGDVEENPGPMALPVTALLLPL |
| | ALLLHAARPDIQMTQSPSSLSASVGDRVTISCRASQDISKYLNWYQQKPGKAPKLLIYHTSRLHS |
| | GVPSRFSGSGSGTDYTFTISSLQPEDIATYFCQQGNTLPYTFGGGTKLEIKGGGGSGGGGSGGGG |
| | SQVQLQESGPGLVKPSETLSLTCTVSGVSLPDYGVSWIRQPPGKGLEWLGVIWGSETTYYNSALK |
| | SRLTISKDTSKSQVSLKLSSVTAADTAVYYCAKHYYYGGSYAMDYWGQGTLVTVSSTTTPAPRPP |
| | TPAPTIASQPLSLRPEACRPAAGGAVHTRGLDFACDIYIWAPLAGTCGVLLLSLVITLYCKRGRK |
| | KLLYIFKQPFMRPVQTTQEEDGCSCRFPEEEEGGCELRVKFSRSADAPAYKQGQNQLYNELNLGR |
| | REEYDVLDKRRGRDPEMGGKPRRKNPQEGLYNELQKDKMAEAYSEIGMKGERRRGKGHDGLYQGL |
| | STATKDTYDALHMQALPPRGSGATNFSLLKQAGDVEENPGPMLVRRGARAGPRMPRGWTALCLLS |
| | LLPSGFMSLDNNGTATPELPTQGTFSNVSTNVSYQETTTPSTLGSTSLHPVSQHGNEATTNITET |
| | TVKFTSTSVITSVYGNTNSSVQSQTSVISTVFTTPANVSTPETTLKPSLSPGNVSDLSTTSTSLA |
| | TSPTKPYTSSSPILSDIKAEIKCSGIREVKLTQGICLEQNKTSSCAEFKKDRGEGLARVLCGEEQ |
| | ADADAGAQVCSLLLAQSEVRPQCLLLVLANRTEISSKLQLMKKHQSDLKKLGILDFTEQDVASHQ |
| | SYSQKTPILLTCPTISILSFFSVALLVILACVLWKKRIKPIVWPSLPDHKKTLEHLCKKPRKNLN |
| | VSFNPESFLDCQIHRVDDIQARDEVEGFLQDTFPQQLEESEKQRLGGDVQSPNCPSEDVVITPES |
| | FGRDSSLTCLAGNVSACDAPILSSSRSLDCRESGKNGPHVYQDLLLSLGTTNSTLPPPFSLQSGI |
| | LTLNPVAQGQPILTSLGSNQEEAYVTMSSFYQNQ* |
| 67 | QVKLQESGGGLVKPGGSLKLSCAASGFTFSSYAMSWVRQTPEKRLEWVATISSGGSYTYYPDSVK |
| | GRFTISRDNAKNTLYLQMSSLRSEDTAMYYCARQDGYYPGWFANWGQGTTVTVSS |
| 68 | DIELTQSPAIMSASLGEEITLTCSASSSVSYMHWYQQKSGTSPKLLIYSTSNLASGVPSRFSGSG |
| | SGTFYSLTISSVEAEDAADYYCHQWSSYTFGGGTKLEIKR |
| 69 | QVQLQESGAELVKPGASVKLSCKASGYTFTSYWMHWVKQRPGQGLEWIGKINPSNGRTNYNEKFK |
| | SKATLTVDKSSSTAYMQLSSLTSEDSAVYYCARGGVYYDLYYYALDYWGQGTTVTVSS |
| 70 | DIELTQSPATLSVTPGDSVSLSCRASQSISNNLHWYQQKSHESPRLLIKSASQSISGIPSRFSGS |
| | GSGTDFTLSINSVETEDFGMYFCQQSNSWPYTFGGGTKLEIKR |
| 71 | EVQLVESGGGLVKPGGSLKLSCAASGLTFSSYAMSWVRQTPEKRLEWVASISSGGFTYYPDSVKG |
| | RFTISRDNARNILYLQMSSLRSEDTAMYYCARDEVRGYLDVWGAGTTVTVSS |
| 72 | AAYKDIQMTQTTSSLSASLGDRVTISCSASQGISNYLNWYQQKPDGTVKLLIYYTSSLHSGVPSR |
| | FSGSGSGTDYSLTISNLEPEDIATYYCQQYSKLPYTFGGGTKLEIKR |
| 73 | DVQLVESGGGLVQPGGSRKLSCAASGFTFSSFGMHWVRQAPEKGLEWVAYISSGSSTLHYADTVK |
| | GRFTISRDNPKNTLFLQMTSLRSEDTAMYYCARWGNYPHYAMDYWGQGTSVTVSS |
| 74 | DIVMTQSPASLAVSLGQRATISCRASKSVSASGYSYMHWYQQKPGQPPKLLIYLASNLESGVPAR |
| | FSGSGSGTDFTLNIHPVEEEDAVTYYCQHSRELPYTFGGGTKLEIK |
| 75 | QIQLVQSGPELKKPGETVKISCKASGYTFTNYGMNWVKQAPGKGLKWMGWINTYNGEPTYADDFK |
| | GRFDFSLETSASTAYLQINNLKNEDTATYFCARRGYYYGSRYGAMDYWGQGTSVTVSS |
| 76 | DIQMTQSPASLSASVGETVTITCRASGNIHNYLAWYQQKQGKSPQLLVYNAKTLADGVPSRFSGS |
| | GSGTQYSLKINSLQPEDFGSYYCQHFWTTPPWTFGGGTKLEIK |
| 77 | EVQLVESGGGLVKPGGSLKLSCAASGLTFSSYAMSWNRQTPEKRLEWVASISSGGFTYYPDSNKG |
| | RFTISRDNARNILYLQMSSLRSEDTAMYYCARDEVRGYLDVWGAGTTVTVSS |
| 78 | AAYKDIQMTQTTSSLSASLGDRVTISCSASQGISNYLNWYQQKPDGTNKLLIYYTSSLHSGVPSR |
| | FSGSGSGTDYSLHSNLEPEDIATYYCQQYSKLPYTFGGGTKLEIKR |

What is claimed is:

1. An engineered immune cell comprising a chimeric antigen receptor (CAR), wherein the CAR comprises:
(i) a first antigen binding domain that specifically binds to CD19, wherein the first antigen binding domain comprises:
(a) a VH1 region, wherein the VH1 region comprises an amino acid sequence having at least 90% identity with any one selected the group consisting of SEQ ID NO. 1-10, and
(b) a VL1 region, wherein the VL1 region comprises an amino acid sequence having at least 90% identity with any one selected the group consisting of SEQ ID NO. 11-17;
(ii) a second antigen binding domain that specifically binds to CD7, and
(iii) a transmembrane domain and an intracellular signaling domain.

2. The engineered immune cell of claim 1, wherein the VH1 region comprises an amino acid sequence having at least 95% identity with any one selected the group consisting of SEQ ID NO. 1-10.

3. The engineered immune cell of claim 1, wherein the VH1 region comprises an amino acid sequence having at least 99% identity with any one selected the group consisting of SEQ ID NO. 1-10.

4. The engineered immune cell of claim 1, wherein the VH1 region comprises an amino acid sequence of any one selected from the group consisting of SEQ ID NO. 1-10.

5. The engineered immune cell of claim 1, wherein the VL1 region comprises an amino acid sequence having at least 95% identity with any one selected the group consisting of SEQ ID NO. 11-17.

6. The engineered immune cell of claim 1, wherein the VL1 region comprises an amino acid sequence having at least 99% identity with any one selected the group consisting of SEQ ID NO. 11-17.

7. The engineered immune cell of claim 1, wherein the VL1 region comprises an amino acid sequence of any one selected the group consisting of SEQ ID NO. 11-17.

8. The engineered immune cell of claim 1, wherein the first antigen binding domain or the second antigen binding domain is a human or humanized antigen binding domain.

9. The engineered immune cell of claim 1, wherein the first antigen binding domain or the second antigen binding domain is a scFv.

10. The engineered immune cell of claim 1, wherein the first antigen binding domain and the second antigen binding domain are arranged, from amino terminus to carboxyl terminus, in one of following patterns:
(i) VL2-VH1-VL1-VH2;
(ii) VH2-VL1-VH1-VL2;
(iii) VL1-VH2-VL2-VH1;
(iv) VH1-VL2-VH2-VL1;
(v) VL2-VL1-VH1-VH2;
(vi) VH2-VH1-VL1-VL2;
(vii) VL1-VL2-VH2-VH1; or
(viii) VH1-VH2-VL2-VL1;
wherein VH1 represents heavy chain variable domain of the first antigen binding domain, VL1 represents light chain variable light domain of the first antigen binding domain, VH2 represents heavy chain variable domain of the second antigen binding domain, and VL2 represents light chain variable domain of the second antigen binding domain.

11. The engineered immune cell of claim 1, wherein the first antigen binding domain and the second antigen binding domain are arranged, from amino terminus to carboxyl terminus, in one of following patterns:
(i) VL2-VH2-VL1-VH1;
(ii) VL2-VH2-VH1-VL1;
(iii) VL1-VH1-VL2-VH2;
(iv) VL1-VH1-VH2-VL2;
(v) VH2-VL2-VL1-VH1;
(vi) VH2-VL2-VH1-VL1;
(vii) VH1-VL1-VL2-VH2; or
(viii) VH1-VL1-VH2-VL2,
wherein VH1 represents heavy chain variable domain of the first antigen binding domain, VL1 represents light chain variable light domain of the first antigen binding domain, VH2 represents heavy chain variable domain of the second antigen binding domain, and VL2 represents light chain variable domain of the second antigen binding domain.

12. The engineered immune cell of claim 1, wherein the CAR further comprises a second transmembrane domain and a second intracellular signaling domain.

13. The engineered immune cell of claim 12, wherein the first antigen binding domain is linked to the intracellular signaling domain via the transmembrane domain and the second antigen binding domain is linked to the second intracellular signaling domain via the second transmembrane domain.

14. The engineered immune cell of claim 12, wherein the first or second transmembrane domain comprises at least a portion of TCR alpha, TCR beta, CD3 zeta, CD3 gamma, CD3 delta, CD3 epsilon, CD2, CD7, CD27, CD28, CD45, CD4, CD5, CD8, CD9, CD16, CD22, CD33, CD3γ, CD64, CD80, CD86, CD134, CD137, CD152, PD-1, or CD154.

15. The engineered immune cell of claim 12, wherein the first or second intracellular signaling domain comprises at least a portion of CD3 zeta, FcR gamma, FcR beta, CD3 gamma, CD3 delta, CD3 epsilon, CD5, CD22, CD79a, CD79b, or CD66d.

16. The engineered immune cell of claim 15, wherein the first or second intracellular signaling domain further comprises a costimulatory domain.

17. The engineered immune cell of claim 16, wherein the costimulatory domain is selected from the group consisting of CD127, CD27, CD28, 4-1BB (CD137), OX40, CD30, CD40, PD-1, ICOS, MyD88, lymphocyte function-associated antigen-1 (LFA-1), CD2, CD7, LIGHT, NKG2C, B7-H3, and a ligand that specifically binds with CD83.

18. The engineered immune cell of claim 1, Wherein the first antigen binding domain and the second antigen binding domain are linked via a linker.

19. The engineered immune cell of claim 18, wherein the linker is a cleavable linker.

20. The engineered immune cell of claim 19, wherein the linker is a self-cleaving peptide.

21. The engineered immune cell of claim 20, wherein the cleavable linker is selected from P2A, T2A, E2A, and F2A.

22. The engineered immune cell of claim 1, wherein the engineered immune cell is a T cell, an NKT cell or an NK cell.

23. The engineered immune cell of claim 22, wherein the T cell is an alpha beta T cell or a gamma delta T cell.

24. The engineered immune cell of claim 1, wherein the engineered immune cell is derived from a stem cell.

25. The engineered immune cell of claim 24, wherein the stem cell is a hematopoietic stem cell (HSC) or an induced pluripotent stem cell (iPSC).

26. The engineered immune cell of claim 1, wherein the engineered immune cell is an autologous cell or an allogeneic cell.

27. The engineered immune cell of claim 1, wherein the engineered immune cell is obtained from a subject having a condition.

28. The engineered immune cell of claim 1, wherein the engineered immune cell is obtained from a healthy donor.

29. The engineered immune cell of claim 1, wherein an endogenous T cell receptor (TCR) of the engineered immune cell is inactivated.

30. The engineered immune cell of claim 29, wherein a gene encoding a subunit of the endogenous TCR is inactivated such that the endogenous TCR is inactivated.

31. The engineered immune cell of claim 30, the subunit is selected from TCRα, TCRβ, CD3ε, CD3δ, CD3γ, and CD3ζ.

32. The engineered immune cell of claim 1, wherein an endogenous CD7 of the engineered immune cell is inactivated or suppressed.

33. The engineered immune cell of claim 32, Wherein a gene encoding the endogenous CD7 of the engineered immune cell is inactivated, or protein expression of the endogenous CD7 of the engineered immune cell is suppressed.

34. The engineered immune cell of claim 1, wherein expression of one or more endogenous human leukocyte antigen (HLA) genes of the cell remains intact.

35. The engineered immune cell of claim 1, wherein expression of one or more endogenous human leukocyte antigen (HLA) genes of the cell is inhibited.

36. The engineered immune cell of claim 1, expression of one or more endogenous human leukocyte antigen (HLA) genes of the cell is inactivated.

37. The engineered immune cell of any one of claims 31-33, wherein the HLA gene comprises HLA-A, HLA-B, HLA-C, HLA-E, HLA-F, HLA-G, HLA-DRA, HLA-DRB, HLA-DQA and HLA-DQB.

38. The engineered immune cell of claim 1, wherein the engineered immune cell further comprises an enhancer moiety capable of enhancing one or more activities of the engineered immune cell.

39. The engineered immune cell of claim 38, wherein the enhancer moiety is configured to constitutively enhance the one or more activities of the engineered immune cell.

40. The engineered immune cell of claim 39, wherein the enhancer moiety is configured to constitutively upregulate one or more intracellular signaling pathways of the engineered immune cell.

41. The engineered immune cell of claim 40, wherein the one or more intracellular signaling pathways are one or more cytokine signaling pathways.

42. The engineered immune cell of claim 40, wherein the enhancer moiety is self-activating through self-oligomerizing.

43. The engineered immune cell of claim 42, wherein the enhancer moiety is self-activating through self-dimerizing.

44. The engineered immune cell of claim 38, wherein the enhancer moiety is a cytokine or a cytokine receptor.

45. The engineered immune cell of claim 44, wherein the enhancer moiety is selected from the group consisting of IL-2, IL-3, IL-4, IL-6, IL-7, IL-8, IL-10, IL-11, IL-12, IL-15, IL-17, IL-18, IL-21, IL-23, PD-1, PD-L1, CD122, CSF1R, CTAL-4, TIM-3, CCL21, CCL19, TGFR beta, receptors for the same, functional fragments thereof, functional variants thereof, and combinations thereof.

46. The engineered immune cell of claim 38, wherein the enhancer moiety functions as a trans-activating factor or a cis-activating factor.

47. The engineered immune cell of claim 38, the enhancer is linked to the CAR via a linker.

48. The engineered immune cell of claim 47, wherein the linker is a cleavable linker.

49. The engineered immune cell of claim 48, wherein the linker is a self-cleaving peptide.

50. The engineered immune cell of claim 49, wherein the cleavable linker is selected from P2A, T2A, E2A, and F2A.

51. The engineered immune cell of claim 1, wherein the engineered immune cell further comprises an inducible cell death moiety capable of effecting death of the engineered immune cell upon contacting the chimeric polypeptide with a cell death activator.

52. The engineered immune cell of claim 51, the inducible cell death moiety is selected from the group consisting of rapaCasp9, iCasp9, HSV-TK, CD20, ΔCD20, mTMPK, ΔCD19, RQR8, Her2t, CD30, BCMA and EGFRt.

53. The engineered immune cell of claim 52, wherein the inducible cell death moiety is EGFRt, and the cell death activator is an antibody or an antigen binding fragment thereof that binds EGFRt.

54. The engineered immune cell of claim 52, wherein the inducible cell death moiety is HSV-TK, and the cell death activator is GCV.

55. The engineered immune cell of claim 52, wherein the inducible cell death moiety is iCasp9, and the cell death activator is AP1903.

56. The engineered immune cell of claim 51, the cell death activator comprises a nucleic acid, a polynucleotide, an amino acid, a polypeptide, lipid, a carbohydrate, a small molecule, an enzyme, a ribosome, a proteasome, a variant thereof, or any combination thereof.

57. The engineered immune cell of claim 51, wherein the enhancer moiety is linked to the inducible cell death moiety.

58. The engineered immune cell of claim 1, wherein said engineered immune cell exhibits enhanced viability while in presence of cells that are heterologous to said engineered immune cell.

59. A composition comprising the engineered immune cell of any one of claims 1-58, and a pharmaceutically acceptable excipient.

60. An isolated polynucleotide encoding the CAR of any one of claims 1-58.

61. A method of delivering an allogeneic cell therapy comprising administering to a subject in need thereof a population of engineered immune cells of any one of claims 1-58.

62. The method of claim 61, wherein an endogenous TCR of the engineered immune cell is functionally inactive.

63. The method of claim 62, wherein the cell reduces GvHD in the subject compared to an additional cell having a functionally active TCR.

64. A method for treating cancer, comprising administering to a patient in need thereof a population of the engineered immune cells of any one of claims 1-58.

65. The method of claim 64, wherein the cancer is lymphoma or leukemia.

66. A kit comprising the engineered immune cells of any one of claims 1-58 or the isolated polynucleotide of claim 60, and an instruction for using the kit.

67. An engineered immune cell comprising a chimeric antigen receptor (CAR), wherein the CAR comprises:
(i) a humanized first antigen binding domain that specifically binds to CD19, wherein the first antigen binding domain comprises:
(a) a VH1 region, wherein the VH1 region comprises an amino acid sequence having a structure of formula I:
FH1-X1-FH2-X2-FH3-X3-FH4 (I)
wherein:
FH1 comprises an amino acid identical to or comprising 1-3 amino acid residue addition, deletion, or substitutions relative to SEQ ID NO. 18;
FH2 comprises an amino acid identical to or comprising 1-3 amino acid residue addition, deletion, or substitutions relative to SEQ ID NO. 19;
FH3 comprises an amino acid identical to or comprising 1-3 amino acid residue addition, deletion, or substitutions relative to SEQ ID NO. 20;
FH4 comprises an amino acid identical to or comprising 1-3 amino acid residue addition, deletion, or substitutions relative to SEQ ID NO. 21; and
X1, X2, and X3 represent heavy chain CDRs of the first antigen binding domain; and
(b) a VL1 region, wherein the VH1 region comprises an amino acid sequence having a structure of formula II:
FL1-Y1-FL2-Y2-FL3-Y3-FL4 (II)
wherein:
FL1 comprises an amino acid identical to or comprising 1-3 amino acid residue addition, deletion, or substitutions relative to SEQ ID NO. 22;
FL2 comprises an amino acid identical to or comprising 1-3 amino acid residue addition, deletion, or substitutions relative to SEQ ID NO. 23;
FL3 comprises an amino acid identical to or comprising 1-3 amino acid residue addition, deletion, or substitutions relative to SEQ ID NO. 24;
FL4 comprises an amino acid identical to or comprising 1-3 amino acid residue addition, deletion, or substitutions relative to SEQ ID NO. 25; and
Y1, Y2, and Y3 represent light chain CDRs of the first antigen binding domain;
(ii) a second antigen binding domain that specifically binds to CD7; and
(iii) a transmembrane domain and an intracellular signaling domain.

68. The engineered immune cell of claim 67, wherein FH1 is selected from a sequence of SEQ ID NO. 18, 34, 35 or 36.

69. The engineered immune cell of claim 67, wherein FH2 is selected from a sequence of SEQ ID NO. 19 or 37.

70. The engineered immune cell of claim 67, wherein FH3 is selected from a sequence of SEQ ID NO. 20, 38, 39, 40, 41, 42, 43 or 44.

71. The engineered immune cell of claim 67, wherein FH4 is selected from a sequence of SEQ ID NO. 21 or 45.

72. The engineered immune cell of claim 67, wherein FL1 is selected from a sequence of SEQ ID NO. 22 or 46.

73. The engineered immune cell of claim 67, wherein FL2 is selected from a sequence of SEQ ID NO. 23, 47 or 48.

74. The engineered immune cell of claim 67, wherein FL3 is selected from a sequence of SEQ ID NO. 24, 49, 50, 51 or 52.

75. The engineered immune cell of claim 67, wherein FL4 is a sequence of SEQ ID NO. 25.

76. The engineered immune cell of claim 67, the FH1 comprises an amino acid identical to or comprising 1-3 amino acid residue addition, deletion, or substitutions relative to SEQ ID NO. 34, the FH2 comprises an amino acid identical to or comprising 1-3 amino acid residue addition, deletion, or substitutions relative to SEQ ID NO. 37, the FH3 comprises an amino acid identical to or comprising 1-3 amino acid residue addition, deletion, or substitutions relative to SEQ ID NO. 38, the FH4 comprises an amino acid identical to or comprising 1-3 amino acid residue addition, deletion, or substitutions relative to SEQ ID NO. 45.

77. The engineered immune cell of claim 67, the FH1 comprises an amino acid identical to or comprising 1-3 amino acid residue addition, deletion, or substitutions relative to SEQ ID NO. 34, the FH2 comprises an amino acid identical to or comprising 1-3 amino acid residue addition, deletion, or substitutions relative to SEQ ID NO. 37, the FH3 comprises an amino acid identical to or comprising 1-3 amino acid residue addition, deletion, or substitutions relative to SEQ ID NO. 39, the FH4 comprises an amino acid identical to or comprising 1-3 amino acid residue addition, deletion, or substitutions relative to SEQ ID NO. 45.

78. The engineered immune cell of claim 67, the FH1 comprises an amino acid identical to or comprising 1-3 amino acid residue addition, deletion, or substitutions relative to SEQ ID NO. 34, the FH2 comprises an amino acid identical to or comprising 1-3 amino acid residue addition, deletion, or substitutions relative to SEQ ID NO. 37, the FH3 comprises an amino acid identical to or comprising 1-3 amino acid residue addition, deletion, or substitutions relative to SEQ ID NO. 40, the FH4 comprises an amino acid identical to or comprising 1-3 amino acid residue addition, deletion, or substitutions relative to SEQ ID NO. 45.

79. The engineered immune cell of claim 67, wherein the FH1 comprises an amino acid identical to or comprising 1-3 amino acid residue addition, deletion, or substitutions relative to SEQ ID NO. 34, the FH2 comprises an amino acid identical to or comprising 1-3 amino acid residue addition, deletion, or substitutions relative to SEQ ID NO. 19, the FH3 comprises an amino acid identical to or comprising 1-3 amino acid residue addition, deletion, or substitutions relative to SEQ ID NO. 40, the FH4 comprises an amino acid identical to or comprising 1-3 amino acid residue addition, deletion, or substitutions relative to SEQ ID NO. 45.

80. The engineered immune cell of claim 67, wherein the FH1 comprises an amino acid identical to or comprising 1-3 amino acid residue addition, deletion, or substitutions relative to SEQ ID NO. 35, the FH2 comprises an amino acid identical to or comprising 1-3 amino acid residue addition, deletion, or substitutions relative to SEQ ID NO. 19, the FH3 comprises an amino acid identical to or comprising 1-3 amino acid residue addition, deletion, or substitutions relative to SEQ ID NO. 41, the FH4 comprises an amino acid identical to or comprising 1-3 amino acid residue addition, deletion, or substitutions relative to SEQ ID NO. 21.

81. The engineered immune cell of claim 67, wherein the FH1 comprises an amino acid identical to or comprising 1-3 amino acid residue addition, deletion, or substitutions relative to SEQ ID NO. 18, the FH2 comprises an amino acid identical to or comprising 1-3 amino acid residue addition, deletion, or substitutions relative to SEQ ID NO. 19, the FH3 comprises an amino acid identical to or comprising 1-3 amino acid residue addition, deletion, or substitutions relative to SEQ ID NO. 20, the FH4 comprises an amino acid identical to or comprising 1-3 amino acid residue addition, deletion, or substitutions relative to SEQ ID NO. 21.

82. The engineered immune cell of claim 67, wherein the FH1 comprises an amino acid identical to or comprising 1-3 amino acid residue addition, deletion, or substitutions relative to SEQ ID NO. 18, the FH2 comprises an amino acid identical to or comprising 1-3 amino acid residue addition, deletion, or substitutions relative to SEQ ID NO. 37, the FH3 comprises an amino acid identical to or comprising 1-3 amino acid residue addition, deletion, or substitutions relative to SEQ ID NO. 42, the FH4 comprises an amino acid identical to or comprising 1-3 amino acid residue addition, deletion, or substitutions relative to SEQ ID NO. 21.

83. The engineered immune cell of claim 67, wherein the FH1 comprises an amino acid identical to or comprising 1-3 amino acid residue addition, deletion, or substitutions relative to SEQ ID NO. 18, the FH2 comprises an amino acid identical to or comprising 1-3 amino acid residue addition, deletion, or substitutions relative to SEQ ID NO. 36, the FH3 comprises an amino acid identical to or comprising 1-3 amino acid residue addition, deletion, or substitutions relative to SEQ ID NO. 43, the FH4 comprises an amino acid identical to or comprising 1-3 amino acid residue addition, deletion, or substitutions relative to SEQ ID NO. 21.

84. The engineered immune cell of claim 67, wherein the FH1 comprises an amino acid identical to or comprising 1-3 amino acid residue addition, deletion, or substitutions relative to SEQ ID NO. 18, the FH2 comprises an amino acid identical to or comprising 1-3 amino acid residue addition, deletion, or substitutions relative to SEQ ID NO. 19, the FH3 comprises an amino acid identical to or comprising 1-3 amino acid residue addition, deletion, or substitutions relative to SEQ ID NO. 44, the FH4 comprises an amino acid identical to or comprising 1-3 amino acid residue addition, deletion, or substitutions relative to SEQ ID NO. 21.

85. The engineered immune cell of claim 67, wherein the FL1 comprises an amino acid identical to or comprising 1-3 amino acid residue addition, deletion, or substitutions relative to SEQ ID NO. 22, the FL2 comprises an amino acid identical to or comprising 1-3 amino acid residue addition, deletion, or substitutions relative to SEQ ID NO. 47, the FL3 comprises an amino acid identical to or comprising 1-3 amino acid residue addition, deletion, or substitutions relative to SEQ ID NO. 49 and the FL4 comprises an amino acid identical to or comprising 1-3 amino acid residue addition, deletion, or substitutions relative to SEQ ID NO. 25.

86. The engineered immune cell of claim 67, wherein the FL1 comprises an amino acid identical to or comprising 1-3 amino acid residue addition, deletion, or substitutions relative to SEQ ID NO. 22, the FL2 comprises an amino acid identical to or comprising 1-3 amino acid residue addition, deletion, or substitutions relative to SEQ ID NO. 47, the FL3 comprises an amino acid identical to or comprising 1-3 amino acid residue addition, deletion, or substitutions relative to SEQ ID NO. 50 and the FL4 comprises an amino acid identical to or comprising 1-3 amino acid residue addition, deletion, or substitutions relative to SEQ ID NO. 25.

87. The engineered immune cell of claim 67, wherein the FL1 comprises an amino acid identical to or comprising 1-3 amino acid residue addition, deletion, or substitutions relative to SEQ ID NO. 22, the FL2 comprises an amino acid identical to or comprising 1-3 amino acid residue addition, deletion, or substitutions relative to SEQ ID NO. 47, the FL3 comprises an amino acid identical to or comprising 1-3 amino acid residue addition, deletion, or substitutions relative to SEQ ID NO. 51 and the FL4 comprises an amino acid identical to or comprising 1-3 amino acid residue addition, deletion, or substitutions relative to SEQ ID NO. 25.

88. The engineered immune cell of claim 67, wherein the FL1 comprises an amino acid identical to or comprising 1-3 amino acid residue addition, deletion, or substitutions relative to SEQ ID NO. 46, the FL2 comprises an amino acid identical to or comprising 1-3 amino acid residue addition, deletion, or substitutions relative to SEQ ID NO. 47, the FL3 comprises an amino acid identical to or comprising 1-3 amino acid residue addition, deletion, or substitutions relative to SEQ ID NO. 24 and the FL4 comprises an amino acid identical to or comprising 1-3 amino acid residue addition, deletion, or substitutions relative to SEQ ID NO. 25.

89. The engineered immune cell of claim 67, wherein the FL1 comprises an amino acid identical to or comprising 1-3 amino acid residue addition, deletion, or substitutions relative to SEQ ID NO. 22, the FL2 comprises an amino acid identical to or comprising 1-3 amino acid residue addition, deletion, or substitutions relative to SEQ ID NO. 23, the FL3 comprises an amino acid identical to or comprising 1-3 amino acid residue addition, deletion, or substitutions relative to SEQ ID NO. 24 and the FL4 comprises an amino acid identical to or comprising 1-3 amino acid residue addition, deletion, or substitutions relative to SEQ ID NO. 25.

90. The engineered immune cell of claim 67, wherein the FL1 comprises an amino acid identical to or comprising 1-3 amino acid residue addition, deletion, or substitutions relative to SEQ ID NO. 46, the FL2 comprises an amino acid identical to or comprising 1-3 amino acid residue addition, deletion, or substitutions relative to SEQ ID NO. 48, the FL3 comprises an amino acid identical to or comprising 1-3 amino acid residue addition, deletion, or substitutions relative to SEQ ID NO. 52 and the FL4 comprises an amino acid identical to or comprising 1-3 amino acid residue addition, deletion, or substitutions relative to SEQ ID NO. 25.
